(19) United States
(12) Patent Application Publication
(10) Pub. No.: US 2025/0011427 A1
(43) Pub. Date: Jan. 9, 2025

(54) ANTI-CD3 ANTIBODIES WITH CROSS-REACTIVITY TO HUMAN AND CYNOMOLGUS PROTEINS

(71) Applicant: CONCEPT TO MEDICINE BIOTECH CO., LTD., Shanghai (CN)

(72) Inventors: Wei Zhen, Shanghai (CN); Liu YANG, Shanghai (CN); Lei FANG, Shanghai (CN)

(21) Appl. No.: 18/700,235

(22) PCT Filed: Oct. 12, 2022
(86) PCT No.: PCT/CN2022/124968

§ 371 Date: Apr. 10, 2024

(30) Foreign Application Priority Data
Oct. 12, 2021 (WO) PCT/CN2021/123371

(51) Int. Cl. C07K 16/28; A61K 39/00; A61P 35/00; C07K 16/30

(52) U.S. Cl. CPC C07K 16/2809; A61P 35/00; C07K 16/2878; C07K 16/30; A61K 2039/505; C07K 2317/31; C07K 2317/33; C07K 2317/565; C07K 2317/622

(57) ABSTRACT

The present disclosure provides new anti-CD3 antibodies, including humanized ones, and their antigen-binding fragments. These antibodies can bind to both human and cynomolgus CD3 at high affinity, and their single chain fragments (scFv) can be readily incorporated into multispecific antibodies to effectively activate T cell in the presence of tumor cells. Further, even though a relatively small number, these antibodies have anti-CD3 activities of a broad range, which are accordingly categorized into nine different grades. As further demonstrated, these anti-CD3 antibodies and fragments can be suitably used in bi- or tri-specific antibodies to achieve optimized activities and safety margins.

### BACKGROUND

CD3 (cluster of differentiation 3) is a protein complex and T cell co-receptor that is involved in activating both the cytotoxic T cell (CD8+ naive T cells) and T helper cells (CD4+ naive T cells). CD3 is composed of four distinct chains. In mammals, the complex contains a CD3γ chain, a CD3δ chain, and two CD3ε chains. These chains associate with the T-cell receptor (TCR) and the CD3-zeta (ζ-chain) to generate an activation signal in T lymphocytes. The TCR, CD3-zeta, and the other CD3 molecules together constitute the TCR complex.

Given its complex extracellular structure, anti-CD3 agonist antibodies are difficult to develop. Over the many decades since the initial attempt to generate therapeutic antibodies targeting CD3, very few have been successful. SP34 is the first widely used anti-CD3 agonist antibody and can cross-react with cynomolgus CD3. Also, a good number of derivatives have been developed based off the original SP34 antibody. Nevertheless, it has proven to be a great challenge to adopt the SP34 antibody or its derivatives into a scFv format. Given that scFv is the most commonly used fragment for incorporation into bi-specific or tri-specific antibody, this deficiency has severely limited the application of SP34. Another widely used anti-CD3 antibody is OKT3, which unfortunately, does not have cross-reactivity to cynomolgus CD3 and thus its clinical use has been hindered by the lack of suitable pre-clinical testing models.

Bispecific antibodies having an anti-CD3 unit provide a targeted immuno-oncology platform that connects patients' own T cells to malignant cells. Such bispecific antibodies ensure direct connection of the T-cell to the cancer cell and thus can enable T-cell activation, which results in cytotoxic activity directed to the cancer cell. The development of bispecific antibodies having a specificity to CD3 has been hampered by the lack of anti-CD3 antibodies with balanced potency, cross-reactivity to cynomolgus, and adoptability into suitable fragment formats.

### SUMMARY

The present disclosure provides new anti-CD3 antibodies, including humanized ones, and their antigen-binding fragments. These antibodies can bind to both human and cynomolgus CD3 at high affinity, and their single chain fragments (scFv) have reasonable developability, so that these CD3 antibodies can be readily incorporated into multispecific antibodies to effectively activate T cell in the presence of tumor cells. Further, with a relatively small number of them, these antibodies exhibited anti-CD3 activities of a broad range. Based on the activities, these antibodies were categorized into nice different grades (Grades 1-9).

Accordingly, in one embodiment, provided is an anti-CD3 antibody or antigen-binding fragment thereof. In some embodiments, the anti-CD3 antibody or antigen-binding fragment thereof has binding affinity to both human and cynomolgus CD3. In some embodiments, the anti-CD3 antibody or antigen-binding fragment thereof comprises a heavy chain variable region (VH) comprising a VH CDR1, VH CDR2, and VH CDR3, and a light chain variable region (VL) comprising a VL CDR1, VL CDR2 and VL CDR3, wherein: the VH CDR1 comprises the amino acid sequence of SEQ ID NO:7, the VH CDR2 comprises the amino acid sequence of SEQ ID NO:8, 64, 65, or 66, the VH CDR3 comprises the amino acid sequence of SEQ ID NO:9, the VL CDR1 comprises the amino acid sequence of SEQ ID NO:10, the VL CDR2 comprises the amino acid sequence of SEQ ID NO:11, and the VL CDR3 comprises the amino acid sequence of SEQ ID NO:12 or 67; the VH CDR1 comprises the amino acid sequence of SEQ ID NO:13, the VH CDR2 comprises the amino acid sequence of SEQ ID NO:14, 68, 69 or 70, the VH CDR3 comprises the amino acid sequence of SEQ ID NO:15, the VL CDR1 comprises the amino acid sequence of SEQ ID NO:16, the VL CDR2 comprises the amino acid sequence of SEQ ID NO:11, and the VL CDR3 comprises the amino acid sequence of SEQ ID NO:17 or 71; or the VH CDR1 comprises the amino acid sequence of SEQ ID NO:13, the VH CDR2 comprises the amino acid sequence of SEQ ID NO:18, 72, 73 or 74, the VH CDR3 comprises the amino acid sequence of SEQ ID NO:19, the VL CDR1 comprises the amino acid sequence of SEQ ID NO:20, the VL CDR2 comprises the amino acid sequence of SEQ ID NO:21 or 75, and the VL CDR3 comprises the amino acid sequence of SEQ ID NO:22 or 76.

In some embodiments, the VH CDR1 comprises the amino acid sequence of SEQ ID NO:7, the VH CDR2 comprises the amino acid sequence of SEQ ID NO:8, 64, 65, or 66, the VH CDR3 comprises the amino acid sequence of SEQ ID NO:9, the VL CDR1 comprises the amino acid sequence of SEQ ID NO:10, the VL CDR2 comprises the amino acid sequence of SEQ ID NO:11, and the VL CDR3 comprises the amino acid sequence of SEQ ID NO:12 or 67.

In some embodiments, the VH comprises an amino acid sequence selected from the group consisting of SEQ ID NO:1 and 23-28, optionally with a G55A substitution, a S61A substitution, or the combination thereof, and the VL comprises an amino acid sequence selected from the group consisting of SEQ ID NO:2 and 30-32, optionally with a S93A substitution, all amino acid positions according to Kabat numbering.

In some embodiments, the VH comprises the amino acid sequence of SEQ ID NO:24 and the VL comprises the amino acid sequence of SEQ ID NO:30; the VH comprises the amino acid sequence of SEQ ID NO:25 and the VL comprises the amino acid sequence of SEQ ID NO:30; or the VH comprises the amino acid sequence of SEQ ID NO:26 and the VL comprises the amino acid sequence of SEQ ID NO:30.

In some embodiments, the VH CDR1 comprises the amino acid sequence of SEQ ID NO:13, the VH CDR2 comprises the amino acid sequence of SEQ ID NO:14, 68, 69 or 70, the VH CDR3 comprises the amino acid sequence of SEQ ID NO:15, the VL CDR1 comprises the amino acid sequence of SEQ ID NO:16, the VL CDR2 comprises the amino acid sequence of SEQ ID NO:11, and the VL CDR3 comprises the amino acid sequence of SEQ ID NO:17 or 71.

In some embodiments, the VH comprises an amino acid sequence selected from the group consisting of SEQ ID NO:3 and 36-41, optionally with a G55A substitution, a S61A substitution, or the combination thereof, and the VL comprises an amino acid sequence selected from the group consisting of SEQ ID NO:4 and 44-46, optionally with a S93A substitution, all amino acid positions according to Kabat numbering.

In some embodiments, the VH comprises the amino acid sequence of SEQ ID NO:37 and the VL comprises the amino acid sequence of SEQ ID NO:45; the VH comprises the amino acid sequence of SEQ ID NO:38 and the VL comprises the amino acid sequence of SEQ ID NO:45; the VH comprises the amino acid sequence of SEQ ID NO:39 and the VL comprises the amino acid sequence of SEQ ID NO:45; the VH comprises the amino acid sequence of SEQ ID NO:37 and the VL comprises the amino acid sequence of SEQ ID NO:46; the VH comprises the amino acid sequence of SEQ ID NO:38 and the VL comprises the amino acid sequence of SEQ ID NO:46; or the VH comprises the amino acid sequence of SEQ ID NO:39 and the VL comprises the amino acid sequence of SEQ ID NO:46.

In some embodiments, the VH comprises the amino acid sequence of SEQ ID NO:37, optionally with a G55A substitution, a S61A substitution, or the combination thereof, and the VL comprises the amino acid sequence of SEQ ID NO:45, optionally with a S93A substitution, all amino acid positions according to Kabat numbering.

In some embodiments, the VH CDR1 comprises the amino acid sequence of SEQ ID NO:13, the VH CDR2 comprises the amino acid sequence of SEQ ID NO:18, 72, 73 or 74, the VH CDR3 comprises the amino acid sequence of SEQ ID NO:19, the VL CDR1 comprises the amino acid sequence of SEQ ID NO:20, the VL CDR2 comprises the amino acid sequence of SEQ ID NO:21 or 75, and the VL CDR3 comprises the amino acid sequence of SEQ ID NO:22 or 76.

In some embodiments, the VH comprises an amino acid sequence selected from the group consisting of SEQ ID NO:5 and 50-55, optionally with a G55A substitution, a S61A substitution, or the combination thereof, and the VL comprises an amino acid sequence selected from the group consisting of SEQ ID NO:6 and 59-62, optionally with a S53A substitution, a S93A substitution, or the combination thereof, all amino acid positions according to Kabat numbering.

In some embodiments, the VH comprises an amino acid sequence of SEQ ID NO:50 and the VL comprises an amino acid sequence of SEQ ID NO:60; or the VH comprises an amino acid sequence of SEQ ID NO:50 and the VL comprises an amino acid sequence of SEQ ID NO:61.

Methods and uses are also provided, for using the disclosed antibodies or fragment for treating diseases such as cancer.

### BRIEF DESCRIPTION OF THE DRAWINGS

FIG. 1 shows that the anti-CD3 chimeric antibodies bound to human PBMC and cynomolgus PBMC with different potency.

FIG. 2 shows that the CD3 activity of the tested chimeric antibodies displayed weaker response compared with the benchmark antibody OKT3.

FIG. 3 shows 4-1BB expression induced by the tested anti-CD3 chimeric antibodies.

FIG. 4 shows cell-based binding of the tested humanized CD3 antibodies.

FIG. 5 shows that the tested humanized CD3 antibodies exhibited a diverse variety of CD3 activities.

FIG. 6 shows 4-1BB expression induced by the humanized CD3 antibodies.

FIG. 7 shows the CD3 NFAT activities examined by TCR/CD3 cells.

FIG. 8 shows the TAA-dependent agonistic activity of CD3 BsAbs, in comparison of SP34.

FIG. 9 shows that 1+1 format bispecific antibody induced more robust response than 2+Lc2 format bispecific antibody.

FIG. 10 shows that 4-1BB-induced TL-2 secretion was dependent on CD3.

FIG. 11 shows that IL-2 secretion stimulated by trispecific antibodies had a positive correlation of CD3 NFAT activity.

FIG. 12 shows that 4-1BB induction stimulated by trispecific antibodies shows a positive correlation of CD3 NFAT activity.

FIG. 13 shows that IL-2 secretion and cell lysis activity induced by benchmark bispecific antibodies.

FIG. 14 shows that IL-2 secretion and cell lysis activity induced by benchmark trispecific antibodies.

FIG. 15 shows the design and results of an in vivo experiment for treating B16F10-h5T4 induced tumor with trispecific antibodies.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein,” “amino acid chain,” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amidation, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

The term “isolated” as used herein with respect to cells, nucleic acids, such as DNA or RNA, refers to molecules separated from other DNAs or RNAs, respectively, that are present in the natural source of the macromolecule. The term “isolated” as used herein also refers to a nucleic acid or peptide that is substantially free of cellular material, viral material, or culture medium when produced by recombinant DNA techniques, or chemical precursors or other chemicals when chemically synthesized. Moreover, an “isolated nucleic acid” is meant to include nucleic acid fragments which are not naturally occurring as fragments and would not be found in the natural state. The term “isolated” is also used herein to refer to cells or polypeptides which are isolated from other cellular proteins or tissues. Isolated polypeptides is meant to encompass both purified and recombinant polypeptides.

As used herein, an “antibody” or “antigen-binding polypeptide” refers to a polypeptide or a polypeptide complex that specifically recognizes and binds to an antigen. An antibody can be a whole antibody and any antigen binding fragment or a single chain thereof. Thus the term “antibody” includes any protein or peptide containing molecule that comprises at least a portion of an immunoglobulin molecule having biological activity of binding to the antigen. Examples of such include, but are not limited to a complementarity determining region (CDR) of a heavy or light chain or a ligand binding portion thereof, a heavy chain or light chain variable region, a heavy chain or light chain constant region, a framework (FR) region, or any portion thereof, or at least one portion of a binding protein.

The terms “antibody fragment” or “antigen-binding fragment”, as used herein, is a portion of an antibody such as F(ab′)₂, F(ab)₂, Fab′, Fab, Fv, scFv and the like. Regardless of structure, an antibody fragment binds with the same antigen that is recognized by the intact antibody. The term “antibody fragment” includes aptamers, spiegelmers, and diabodies. The term “antibody fragment” also includes any synthetic or genetically engineered protein that acts like an antibody by binding to a specific antigen to form a complex.

A “single-chain variable fragment” or “scFv” refers to a fusion protein of the variable regions of the heavy (V_{H}) and light chains (V_{L}) of immunoglobulins. In some aspects, the regions are connected with a short linker peptide of ten to about 25 amino acids. The linker can be rich in glycine for flexibility, as well as serine or threonine for solubility, and can either connect the N-terminus of the VH with the C-terminus of the V_{L}, or vice versa. This protein retains the specificity of the original immunoglobulin, despite removal of the constant regions and the introduction of the linker. ScFv molecules are known in the art and are described, e.g., in U.S. Pat. No. 5,892,019.

The term antibody encompasses various broad classes of polypeptides that can be distinguished biochemically. Those skilled in the art will appreciate that heavy chains are classified as gamma, mu, alpha, delta, or epsilon (γ, μ, α, δ, ε) with some subclasses among them (e.g., γ1-γ4). It is the nature of this chain that determines the “class” of the antibody as IgG, IgM, IgA IgG, or IgE, respectively. The immunoglobulin subclasses (isotypes) e.g., IgG₁, IgG₂, IgG₃, IgG₄, IgG₅, etc. are well characterized and are known to confer functional specialization. Modified versions of each of these classes and isotypes are readily discernable to the skilled artisan in view of the instant disclosure and, accordingly, are within the scope of the instant disclosure. All immunoglobulin classes are clearly within the scope of the present disclosure, the following discussion will generally be directed to the IgG class of immunoglobulin molecules. With regard to IgG, a standard immunoglobulin molecule comprises two identical light chain polypeptides of molecular weight approximately 23,000 Daltons, and two identical heavy chain polypeptides of molecular weight 53,000-70,000. The four chains are typically joined by disulfide bonds in a “Y” configuration wherein the light chains bracket the heavy chains starting at the mouth of the “Y” and continuing through the variable region.

Antibodies, antigen-binding polypeptides, variants, or derivatives thereof of the disclosure include, but are not limited to, polyclonal, monoclonal, multispecific, human, humanized, primatized, or chimeric antibodies, single chain antibodies, epitope-binding fragments, e.g., Fab, Fab′ and F(ab′)₂, Fd, Fvs, single-chain Fvs (scFv), single-chain antibodies, disulfide-linked Fvs (sdFv), fragments comprising either a VK or VH domain, fragments produced by a Fab expression library, and anti-idiotypic (anti-Id) antibodies (including, e.g., anti-CD3 antibodies disclosed herein). Immunoglobulin or antibody molecules of the disclosure can be of any type (e.g., IgG, IgE, IgM, IgD, IgA, and IgY), class (e.g., IgG1, IgG2, IgG3, IgG4, IgA1 and IgA2) or subclass of immunoglobulin molecule.

Light chains are classified as either kappa or lambda (K, λ). Each heavy chain class may be bound with either a kappa or lambda light chain. In general, the light and heavy chains are covalently bonded to each other, and the “tail” portions of the two heavy chains are bonded to each other by covalent disulfide linkages or non-covalent linkages when the immunoglobulins are generated either by hybridomas, B cells or genetically engineered host cells. In the heavy chain, the amino acid sequences run from an N-terminus at the forked ends of the Y configuration to the C-terminus at the bottom of each chain.

Both the light and heavy chains are divided into regions of structural and functional homology. The terms “constant” and “variable” are used functionally. In this regard, it will be appreciated that the variable domains of both the light (VK) and heavy (VH) chain portions determine antigen recognition and specificity. Conversely, the constant domains of the light chain (CK) and the heavy chain (CH1, CH2 or CH3) confer important biological properties such as secretion, transplacental mobility, Fc receptor binding, complement binding, and the like. By convention the numbering of the constant region domains increases as they become more distal from the antigen-binding site or amino-terminus of the antibody. The N-terminal portion is a variable region and at the C-terminal portion is a constant region; the CH3 and CK domains actually comprise the carboxy-terminus of the heavy and light chain, respectively.

As indicated above, the variable region allows the antibody to selectively recognize and specifically bind epitopes on antigens. That is, the VK domain and VH domain, or subset of the complementarity determining regions (CDRs), of an antibody combine to form the variable region that defines a three dimensional antigen-binding site. This quaternary antibody structure forms the antigen-binding site present at the end of each arm of the Y. More specifically, the antigen-binding site is defined by three CDRs on each of the VH and VK chains (i.e. CDR-H1, CDR-H2, CDR-H3, CDR-L1, CDR-L2 and CDR-L3). In some instances, e.g., certain immunoglobulin molecules derived from camelid species or engineered based on camelid immunoglobulins, a complete immunoglobulin molecule may consist of heavy chains only, with no light chains. See, e.g., Hamers-Casterman et al., Nature 363:446-448 (1993).

In naturally occurring antibodies, the six “complementarity determining regions” or “CDRs” present in each antigen-binding domain are short, non-contiguous sequences of amino acids that are specifically positioned to form the antigen-binding domain as the antibody assumes its three dimensional configuration in an aqueous environment. The remainder of the amino acids in the antigen-binding domains, referred to as “framework” regions, show less inter-molecular variability. The framework regions largely adopt a β-sheet conformation and the CDRs form loops which connect, and in some cases form part of, the β-sheet structure. Thus, framework regions act to form a scaffold that provides for positioning the CDRs in correct orientation by inter-chain, non-covalent interactions. The antigen-binding domain formed by the positioned CDRs defines a surface complementary to the epitope on the immunoreactive antigen. This complementary surface promotes the non-covalent binding of the antibody to its cognate epitope. The amino acids comprising the CDRs and the framework regions, respectively, can be readily identified for any given heavy or light chain variable region by one of ordinary skill in the art, since they have been precisely defined (see “Sequences of Proteins of Immunological Interest,” Kabat, E., et al., U.S. Department of Health and Human Services, (1983); and Chothia and Lesk, J. Mol. Biol., 196:901-917 (1987)).

In the case where there are two or more definitions of a term which is used and/or accepted within the art, the definition of the term as used herein is intended to include all such meanings unless explicitly stated to the contrary. A specific example is the use of the term “complementarity determining region” (“CDR”) to describe the non-contiguous antigen combining sites found within the variable region of both heavy and light chain polypeptides. This particular region has been described by Kabat et al., U.S. Dept. of Health and Human Services, “Sequences of Proteins of Immunological Interest” (1983) and by Chothia et al., J. Mol. Biol. 196:901-917 (1987), which are incorporated herein by reference in their entireties. The CDR definitions according to Kabat and Chothia include overlapping or subsets of amino acid residues when compared against each other. Nevertheless, application of either definition to refer to a CDR of an antibody or variants thereof is intended to be within the scope of the term as defined and used herein. The appropriate amino acid residues which encompass the CDRs as defined by each of the above cited references are set forth in the table below as a comparison. The exact residue numbers which encompass a particular CDR will vary depending on the sequence and size of the CDR. Those skilled in the art can routinely determine which residues comprise a particular CDR given the variable region amino acid sequence of the antibody.

| | Kabat | Chothia |
|---|---|---|
| CDR-H1 | 31-35 | 26-32 |
| CDR-H2 | 50-65 | 52-58 |
| CDR-H3 | 95-102 | 95-102 |
| CDR-L1 | 24-34 | 26-32 |
| CDR-L2 | 50-56 | 50-52 |
| CDR-L3 | 89-97 | 91-96 |

Kabat et al. also defined a numbering system for variable domain sequences that is applicable to any antibody. One of ordinary skill in the art can unambiguously assign this system of “Kabat numbering” to any variable domain sequence, without reliance on any experimental data beyond the sequence itself. As used herein, “Kabat numbering” refers to the numbering system set forth by Kabat et al., U.S. Dept. of Health and Human Services, “Sequence of Proteins of Immunological Interest” (1983).

In addition to table above, the Kabat number system describes the CDR regions as follows: CDR-H1 begins at approximately amino acid 31 (i.e., approximately 9 residues after the first cysteine residue), includes approximately 5-7 amino acids, and ends at the next tryptophan residue. CDR-H2 begins at the fifteenth residue after the end of CDR-H1, includes approximately 16-19 amino acids, and ends at the next arginine or lysine residue. CDR-H3 begins at approximately the thirty third amino acid residue after the end of CDR-H2; includes 3-25 amino acids; and ends at the sequence W-G-X-G, where X is any amino acid. CDR-L1 begins at approximately residue 24 (i.e., following a cysteine residue); includes approximately 10-17 residues; and ends at the next tryptophan residue. CDR-L2 begins at approximately the sixteenth residue after the end of CDR-L1 and includes approximately 7 residues. CDR-L3 begins at approximately the thirty third residue after the end of CDR-L2 (i.e., following a cysteine residue); includes approximately 7-11 residues and ends at the sequence F or W-G-X-G, where X is any amino acid.

Antibodies disclosed herein may be from any animal origin including birds and mammals. Preferably, the antibodies are human, murine, donkey, rabbit, goat, guinea pig, camel, llama, horse, or chicken antibodies. In another embodiment, the variable region may be condricthoid in origin (e.g., from sharks).

As used herein, the term “heavy chain constant region” includes amino acid sequences derived from an immunoglobulin heavy chain. A polypeptide comprising a heavy chain constant region comprises at least one of: a CH1 domain, a hinge (e.g., upper, middle, and/or lower hinge region) domain, a CH2 domain, a CH3 domain, or a variant or fragment thereof. For example, an antigen-binding polypeptide for use in the disclosure may comprise a polypeptide chain comprising a CH1 domain; a polypeptide chain comprising a CH1 domain, at least a portion of a hinge domain, and a CH2 domain; a polypeptide chain comprising a CH1 domain and a CH3 domain; a polypeptide chain comprising a CH1 domain, at least a portion of a hinge domain, and a CH3 domain, or a polypeptide chain comprising a CH1 domain, at least a portion of a hinge domain, a CH2 domain, and a CH3 domain. In another embodiment, a polypeptide of the disclosure comprises a polypeptide chain comprising a CH3 domain. Further, an antibody for use in the disclosure may lack at least a portion of a CH2 domain (e.g., all or part of a CH2 domain). As set forth above, it will be understood by one of ordinary skill in the art that the heavy chain constant region may be modified such that they vary in amino acid sequence from the naturally occurring immunoglobulin molecule.

The heavy chain constant region of an antibody disclosed herein may be derived from different immunoglobulin molecules. For example, a heavy chain constant region of a polypeptide may comprise a CH1 domain derived from an IgG₁ molecule and a hinge region derived from an IgG₃ molecule. In another example, a heavy chain constant region can comprise a hinge region derived, in part, from an IgG₁ molecule and, in part, from an IgG₃ molecule. In another example, a heavy chain portion can comprise a chimeric hinge derived, in part, from an IgG₁ molecule and, in part, from an IgG₄ molecule.

As used herein, the term “light chain constant region” includes amino acid sequences derived from antibody light chain. Preferably, the light chain constant region comprises at least one of a constant kappa domain or constant lambda domain.

By “specifically binds” or “has specificity to,” it is generally meant that an antibody binds to an epitope via its antigen-binding domain, and that the binding entails some complementarity between the antigen-binding domain and the epitope. According to this definition, an antibody is said to “specifically bind” to an epitope when it binds to that epitope, via its antigen-binding domain more readily than it would bind to a random, unrelated epitope. The term “specificity” is used herein to qualify the relative affinity by which a certain antibody binds to a certain epitope. For example, antibody “A” may be deemed to have a higher specificity for a given epitope than antibody “B,” or antibody “A” may be said to bind to epitope “C” with a higher specificity than it has for related epitope “D.”

As used herein, the terms “treat” or “treatment” refer to both therapeutic treatment and prophylactic or preventative measures, wherein the object is to prevent or slow down (lessen) an undesired physiological change or disorder, such as the progression of cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

By “subject” or “individual” or “animal” or “patient” or “mammal,” is meant any subject, particularly a mammalian subject, for whom diagnosis, prognosis, or therapy is desired. Mammalian subjects include humans, domestic animals, farm animals, and zoo, sport, or pet animals such as dogs, cats, guinea pigs, rabbits, rats, mice, horses, cattle, cows, and so on.

As used herein, phrases such as “to a patient in need of treatment” or “a subject in need of treatment” includes subjects, such as mammalian subjects, that would benefit from administration of an antibody or composition of the present disclosure used, e.g., for detection, for a diagnostic procedure and/or for treatment.

### T-Cell Engagers of Improved Therapeutic Index

CD3 (cluster of differentiation 3) is a protein complex and T cell co-receptor that is involved in activating both the cytotoxic T cell (CD8⁺ naive T cells) and T helper cells (CD4⁺ naive T cells). CD3 is composed of four distinct chains. In mammals, the complex contains a CD3γ chain, a CD3δ chain, and two CD3ε chains. These chains associate with the T-cell receptor (TCR) and the CD3-zeta (ζ-chain) to generate an activation signal in T lymphocytes. The TCR, CD3-zeta, and the other CD3 molecules together constitute the TCR complex.

Given its complex extracellular structure, anti-CD3 agonist antibodies are difficult to develop. Over the many decades since the initial attempt to generate therapeutic antibodies targeting CD3, very few have been successful. SP34 is the first widely used anti-CD3 agonist antibody and can cross-react with cynomolgus CD3. Also, a good number of derivatives have been developed based off the original SP34 antibody. Nevertheless, it has proven to be a great challenge to adopt the SP34 antibody or its derivatives into a scFv format. Given that scFv is the most used fragment for incorporation into bi-specific or tri-specific antibodies, this deficiency has severely limited the clinical applications of SP34. Another widely used anti-CD3 antibody is OKT3, which unfortunately does not have cross-reactivity to cynomolgus CD3 and thus its clinical use has been hindered by the lack of suitable pre-clinical testing models.

Current therapeutic uses of anti-CD3 antibodies are frequently associated with off-target toxicities due to CD3 activation in the absence of targeted tumor cells. This challenge is further complicated by the limited availability of anti-CD3 antibodies with different activity/physical and chemical property/safety profiles.

In this context, in one embodiment, the present disclosure provides new anti-CD3 antibodies, including humanized ones, and their antigen-binding fragments. Three mouse hybridoma clones were generated, 153A6B1, 155A9B1, and 192A7B9 (Table 1). Upon humanization, each of the VH/VL resulted in between 2 and 6 humanized versions (Tables 4-6). The combinations of these VH/VL sequences led to the production of fewer than 70 antibodies (Tables 4A-6A).

Unexpectedly, these ˜70 antibodies exhibited vastly different in vitro functional activities, as measured by a nuclear factor of activated T cells (NFAT) luciferase reporter assay, or the 4-1BB induction assay. Based on the NFAT activities or the 4-1BB induction activities, these antibodies were categorized into nine different grades (Table 10, Grades 1-9).

Several of these anti-CD3 antibodies were used to construct bispecific and trispecific antibodies (bispecific or trispecific T-cell engagers). T cells require two signals to become fully activated. The first signal, which is antigen-specific, is provided through the T cell receptor (TCR) which interacts with peptide-MHC molecules on the membrane of antigen presenting cells (APC). The second signal, the co-stimulatory signal, is antigen nonspecific and is provided by the interaction between co-stimulatory molecules expressed on the membrane of APC and the T cell. 4-1BB (CD137, tumor necrosis factor receptor superfamily 9) is a member of TNF-receptor superfamily (TNFRSF) and is an important costimulatory molecule which is expressed following the activation of immune cells, both innate and adaptive immune cells.

It is contemplated that tri-specific T cell engagers that have specificities to CD3, a tumor-associated antigen (TAA), and 4-1BB can have at least the following advantages. First, such a tri-specific antibody can simultaneously provide the first (TCR signal) and second (co-stimulatory) activation signaling to fully, and persistently and efficiently, activate T cells and further shape memory T cell response, which in turn tackles malignant cells. Second, 4-1BB is an inducible costimulatory molecule expressed on activated or tumor infiltrating T and NK cells. The CD3 signal can further synergize with the 4-1BB signal by inducing 4-1BB expression on T cells. Third, if a TAA-dependent anti-4-1BB antibody (i.e., an anti-4-1BB antibody not having agonist activity without the presence of a corresponding TAA) is used, the tri-specific T cell engager would be even safer in non-tumor environments. Finally, the CD3 activity in the tri-specific antibodies can be adjusted (e.g., by using an anti-CD3 portion of the appropriate grade, e.g., Table 9) to fine-tune 4-1BB activation. Likewise, anti-CD3 antibodies of different potencies can also be optimally used in bispecific T cell engagers that further target a tumor-associated antigen (TAA).

For such tri-specific T cell engagers, it is therefore important to identify anti-CD3 units having balanced potency which can better synergize with the 4-1BB signal. The suitable anti-CD3 unit can efficiently activate T cells in the presence of tumor antigen and cannot activate the T cells in the absence of tumor antigen, e.g., as evident by IL2 activation and 4-1BB induction and killing of TAA positive target cell. With such a suitable anti-CD3 unit, the tri-specific T cell engager can have strong 4-1BB activation potency in the presence of a corresponding TAA, and a low (e.g., <10% 4-1BB positive cells) or no 4-1BB activation potency in the absence of the corresponding TAA.

It was demonstrated that anti-CD3/anti-4-1BB/anti-Claudin 18.2 trispecific antibodies with Grade-4 antibodies (anti-CD3 antibodies with marginal in vitro agonist activity, such as Hu153A6B1-2, -3, and -4, Hu155A9B1-8, -9, -10, -14, -15 and -16, and Hu192A7B9-7 and -13) induced sufficient cytokine release and 4-1BB induction in Claudin 18.2 positive cells, without unspecific activation in control cells. Meanwhile, Grade 3 antibodies (e.g., Hu153A6B1-7 and 13, Hu155A9B1-2, -3 and -4, and Hu192A7B9-4) also induced acceptable cytokine release and 4-1BB induction in Claudin 18.2 positive cells, without unspecific activation in TAA negative control cells.

Accordingly, these well balanced anti-CD3 antibodies were subjected to additional testing and exhibited the best efficacy and therapeutic index, when incorporated into bi-specific and tri-specific formats. These data, therefore, demonstrate that relatively weak anti-CD3 antibodies can be excellent candidates for incorporation into bi-specific and tri-specific T cell engagers. Also important, anti-CD3 antibodies of different potencies may be suitable for different scenarios. The presently obtained antibodies, therefore, can be broad clinical uses, enabling off-the-shelf immuno-oncotherapeutic applications.

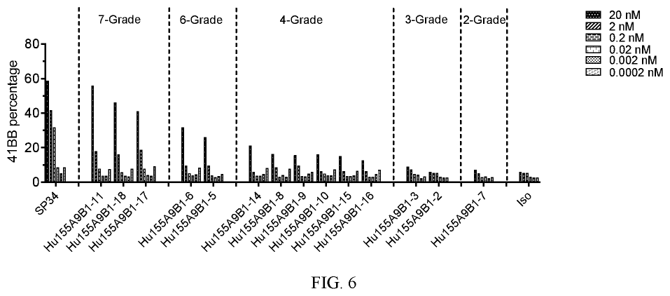

Moreover, different structures of the bi- and tri-specific antibodies have been tested. One of them, the 1+1 format, includes an anti-CD3 unit in a single chain fragment (scFv) and an anti-TAA unit in a regular Fab (VH/VL) domain. The other structure, the 2+Lc2 format, is symmetrical and includes two anti-CD3 scFv fused to the C-terminus of each of the two light chains of the pair of Fab having specificity to the TAA. For a trispecific antibody that further has a specificity to 4-1BB, the anti-4-1BB unit may include two nanobodies fused to the C-terminus of each of the Fc domain of the anti-TAA/CD3 bispecific unit (see illustrations in FIGS. 6 and 10A). It is discovered herein that the 1+1 format has better safety margin and thus is the preferred one; while format 2+Lc2 is nevertheless acceptable as well.

In accordance with one embodiment of the present disclosure, therefore, provided is a multispecific antibody that includes an anti-CD3 unit and an anti-tumor-associated antigen (TAA) unit. The multispecific antibody can be bispecific (anti-CD3 and anti-TAA), trispecific (anti-CD3, anti-TAA and anti-4-1BB or another costimulatory factor), or quadspecific, without limitation.

Anti-CD3 Units with Balanced T-Cell Activation Activity

In a preferred embodiment, a T cell engager of the present disclosure includes an anti-CD3 unit that has a “well-balanced T cell activation activity” (or simply “balanced potency”). An example of a well-balanced T cell activation activity is represented by Grade 3 or 4 as demonstrated in the accompanying experimental examples and as further described below.

The potency of an anti-CD3 unit (e.g., antibody or fragment) can be measured with various different assays known in the art and in different formats. In some embodiments, the assay is a 4-1BB induction assay. In some embodiments, the assay is a T cell proliferation assay. In some embodiments, the assay is an NFAT (nuclear factor of activated T cell) signaling assay.

### A. T Cell Activation NFAT Assay

In one example, the T cell activation activity is measured with an NFAT (nuclear factor of activated T cell) assay. In such an assay, the T cell activation activity may be expressed as the maximum effect (Emax, which is the highest luminescence readout from the assay). An example NFAT assay is performed as follows. An anti-CD3 antibody or antigen-binding fragment (concentration range: 1 nM to 100 nM) is incubated, for 6 hours, with 2.5×10⁴ Jurkat T cells (e.g., from Promega, CAT #J1601) that contain a reporter gene regulated by the NFAT response element (NFAT-RE). Expression of the reporter gene is measured as a luminescence readout, and its maximum value is recorded as Emax, which is used to represent the T cell activation activity of the anti-CD3 antibody or fragment.

For each anti-CD3 antibody or fragment, two Emax values can be obtained. One of them, referred to as “TAA-dependent Emax” or “Emax_{(TAA+)}”, is measured when the test antibody also includes an antibody unit targeting a tumor associated antigen (TAA), and the Jurkat T cells are mixed with TAA-expressing cells (e.g., CHO cells or other tumor cells). In some embodiments, the cell number ratio between the T cells and TAA-expressing cells is 1:1, or alternatively 1:2, 2:1, 1:3, 3:1, 1:4 or 4:1. In some embodiments, such an antibody is a bispecific (CD3+TAA) antibody. In some embodiments, such as antibody is a trispecific (e.g., CD3+TAA+4-1BB) antibody. Non-limiting examples of tumor antigens include Claudin 18.2, 5T4, GPC3, EGFR, Her2, EpCAM, CD20, CD30, CD33, CD47, CD52, CD133, CD73, CEA, gpA33, Mucins, TAG-72, CIX, PSMA, folate-binding protein, GD2, GD3, GM2, VEGF, VEGFR, Integrin, αVβ3, α5β1, ERBB2, ERBB3, MET, IGF1R, EPHA3, TRAILR1, TRAILR2, RANKL, FAP and Tenascin. In some embodiments, the TAA is Claudin 18.2. In some embodiments, the TAA is 5T4.

The second Emax value, referred to as “TAA-free Emax” or “Emax_{(TAA-)}”, is measured when the test antibody does not include the additional anti-TAA unit, when the incubation does not include TAA-expressing cells, or the two TAA's are different.

To enhance cross-platform reproducibility, a reference anti-CD3 antibody can be used, and the activity can be expressed as a percentage over that of the reference antibody. An example reference anti-CD3 antibody is SP34 (see sequences in Table 2B, SEQ ID NO:78 and 79), in a conventional full-size, monospecific Fab format. As such, the NFAT assay conditions (e.g., Jurkat T cell numbers) can vary without impacting the final results (% over SP34 activity). In a preferred embodiment, for the measurement of TAA-free Emax, the test anti-CD3 antibody is bivalent, and the reference antibody (e.g., SP34) is also bivalent. For instance, both are in the conventional full-size Fab antibody format. In some embodiment, for the measurement of TAA-free Emax, the test anti-CD3 antibody and the reference antibody (e.g., SP34) are in the same format, such as full-size Fab antibody, scFv, or multi-specific antibody. Such preferences, however, do not apply to the measurement of TAA-dependent Emax, in some embodiments.

In some embodiments, an anti-CD3 antibody or antigen-binding fragment of the present disclosure has a balanced T cell activation activity which, when measured with the NFAT assay, meets the following criteria: (A) Emax_{(TAA-)}<50% of Emax_{(TAA-)} of SP34, and (B) Emax_{(TAA+)}>Emax_{(TAA-)} of SP34. In some embodiments, for criteria (A), the Emax_{(TAA-)} is less than 10%, 15%, 20%, 25%, 30%, 35%, 40%, 45%50%, 55%, 60%, 65% or 70% of Emax_{(TAA-)} of SP34, while in (B) Emax_{(TAA+)}>Emax_{(TAA-)} of SP34. In some embodiments, for criteria (A), the Emax_{(TAA-)} is greater than 1%, 2%, 3% 4%, 5%, 6%, 7% 8%, 9% 10%, 11%, 12%, 13%, 14%, 15%, or 20% and but less than 10%, 15%, 20%, 25%, 30%, 35%, 40%, 45%, 50%, 55%, 60%, 65% or 70% of Emax_{(TAA-)} of SP34, while in (B) Emax_{(TAA+)}>Emax_{(TAA-)} of SP34.

### B. Alternative T Cell Activation Assay

In some embodiments, the T cell activation activity of an anti-CD3 unit can be measured with the unit, or the entire multispecific antibody, in an NFAT assay such as those described in the examples. In some embodiments, the activity is expressed as the EC50 from the NFAT assay.

In some embodiments, an EC50 greater than 10 μg/mL is considered a balanced T cell activation activity when the measurement is carried out in the absence of cells expressing the TAA (EC50_{TAA-}). In some embodiments, for an anti-CD3 antibody or fragment having a balanced T cell activation activity, the EC50 or EC50_{TAA-} is greater than 5 μg/mL, 10 μg/mL, 20 μg/mL, 30 μg/mL, 40 μg/mL, 50 μg/mL, 100 μg/mL, 200 μg/mL, or 300 μg/mL. In some embodiments, for an anti-CD3 antibody or fragment having a balanced T cell activation activity, the EC50_{TAA-} is less than 50 μg/mL, 100 μg/mL, 200 μg/mL, 300 μg/mL, 400 μg/mL, 500 μg/mL, 1000 μg/mL, or 5000 μg/mL.

In some embodiments, the anti-CD3 unit (or the entire multispecific antibody) has a T cell activation activity, EC50, of greater than 10 nM. In some embodiments, the measurement is carried out in the absence of cells expressing the TAA (EC50_{TAA-}). In some embodiments, for an anti-CD3 antibody or fragment having a balanced T cell activation activity, the EC50 or EC50_{TAA-} is greater than 5 nM, 10 nM, 20 nM, 30 nM, 40 nM, 50 nM, 100 nM, 200 nM, or 300 nM. In some embodiments, for an anti-CD3 antibody or fragment having a balanced T cell activation activity, the EC50_{TAA-} is less than 50 nM, 100 nM, 200 nM, 300 nM, 400 nM, 500 nM, 1000 nM, or 5000 nM.

In some embodiments, an anti-CD3 unit is considered as having a balanced T cell activation when it has a TAA-dependent T cell activation activity EC50_{TAA+} that is at least 5-fold lower than the EC50_{TAA-}. In some embodiments, the EC50_{TAA+} is at least 6-fold, 7-fold, 8-fold, 9-fold, 10-fold, 11-fold, 12-fold, 13-fold, 14-fold, 15-fold, 20-fold, 25-fold, 30-fold, 40-fold, 50-fold, 100-fold, 200-fold, 500-fold or 1000-folder lower than the EC50_{TAA-}.

In some embodiments, when measuring the EC50_{TAA+}, the cell number ratio between the T cells and TAA-expressing cells is 1:1, or alternatively 1:2, 2:1, 1:3, 3:1, 1:4 or 4:1.

In some embodiments, the EC50_{TAA-} is lower than 100 μg/mL, 90 μg/mL, 80 μg/mL, 70 μg/mL, 60 μg/mL, 50 μg/mL, 40 μg/mL, 30 μg/mL, 20 μg/mL, 15 μg/mL, 10 μg/mL, 9 μg/mL, 8 μg/mL, 7 μg/mL, 6 μg/mL, 5 μg/mL, 4 μg/mL, 3 μg/mL, 2 μg/mL, 1 μg/mL, 0.5 μg/mL, or 0.1 μg/mL. In some embodiments, the EC50_{TAA-} is lower than 100 nM, 90 nM, 80 nM, 70 nM, 60 nM, 50 nM, 40 nM, 30 nM, 20 nM, 15 nM, 10 nM, 9 nM, 8 nM, 7 nM, 6 nM, 5 nM, 4 nM, 3 nM, 2 nM, 1 nM, 0.5 nM, or 0.1 nM.

### C. 4-1BB Induction Assay

In some embodiments, the T cell activation activity of an anti-CD3 unit is measured with a 4-1BB induction assay. In one embodiment, the 4-1BB induction assay is conducted with a monoclonal/monospecific anti-CD3 antibody of the conventional format.

The 4-1BB induction activity of a monoclonal/monospecific anti-CD3 antibody of the conventional format can be determined as follows. Primary human PBMC at a density of 1×10⁵ cells per well can be cultured in a 96-well plate. Human PBMC cells typically include about 30% CD8⁺ cells which are most relevant to the assay. Tested antibodies can be serially diluted and added to a 96-well plate, at a final concentration starting from, e.g., 20 nM. Following a 48 hours incubation at 37° C., the cells can be collected. After washing by PBS, the samples can be stained using standard procedures by incubation in the dark at room temperature for 30 minutes with the following antibodies: anti-human CD4-APC (Ebiosciene, 17-0048-42), anti-human CD8-BV510 (BD bioscience, 563919), anti-human 4-1BB-PE (BD Pharmingen, 555956). The samples can then be washed twice with FACS buffer. After centrifugation, the supernatant is discarded, and the cells are resuspended in 0.2 mL FACS buffer. The 4-1BB⁺ CD8⁺ T cell subsets amongst CD8⁺ T cells can be evaluated by MACSQuant Analyzer 16. The percentage of 4-1BB⁺ CD8⁺ T cells over total CD8⁺ T cells (%) is used to indicate the 4-1BB induction rate:

$$4 - 1 BB Induction Rate ( \% ) = \frac{\# of 4 - 1 {BB}^{+} CD {8}^{+} cells}{\# of total CD {8}^{+}} \% .$$

More briefly, the 4-1BB induction activity of a monospecific anti-CD3 antibody is measured by incubating human peripheral blood CD8+ cells with the monospecific antibody that includes the anti-CD3 unit at a final concentration of, e.g., 20 nM, at 37° C. for 48 hours, and measuring the percentage of 4-1BB+ CD8+ cells over the total number of CD8+ cells as the 4-1BB induction rate. In some embodiments, the monospecific anti-CD3 antibody is bi-valent. In some embodiments, the monospecific anti-CD3 antibody has a conventional Fab plus Fc format.

The final concentration of the antibody can be 20 nM, as illustrated above, or alternatively 1 nM, 5 nM, 10 nM, 15 nM, 20 nM, 30 nM, 40 nM, 50 nM or 100 nM, without limitation. In a preferred embodiment, the concentration is 10 nM or 20 nM.

The above example the 4-1BB induction activity measurement procedure uses primary human peripheral blood cells from donor individuals. In some embodiments, alternative cells, such as CD8⁺ cells from established cell lines, can be used.

While the above 4-1BB induction assay is illustrated to measure the T cell activation activity of monospecific anti-CD3 antibodies, it can be also used for bispecific or trispecific/multi-specific antibodies. With a bispecific or trispecific/multi-specific antibody that further includes an anti-TAA unit, the 4-1BB induction activity can be a TAA-dependent 4-1BB induction activity (e.g., co-incubation with TAA-expressing cells), or a TAA-free 4-1BB induction activity (e.g., no co-incubation with TAA-expressing cells).

In some embodiments, these TAA-free or TAA-dependent dependent 4-1BB induction rates are measured when the multispecific antibody does not have an anti-4-1BB unit. In some embodiments, these TAA-free or TAA-dependent dependent 4-1BB induction rates are measured when the multispecific antibody further includes an anti-4-1BB unit.

In some embodiments, the measurement is made with a multispecific antibody of the 1+1 format. In some embodiments, the measurement is made with a multispecific antibody of the 2+2Lc format. In some embodiments, the measurement is made with a trispecific antibody of the present disclosure. In some embodiments, the trispecific antibody includes an anti-CD3 unit, an anti-TAA unit, and an anti-4-1BB unit. In some embodiments, the anti-4-1BB unit includes one or two TAA-dependent agonist antibodies or fragments.

The TAA-free and TAA-dependent 4-1BB induction rates of the multispecific antibody can be measured with a method similar to that for a monospecific antibody. For instance, human PBMC at a density of 1×10⁵ cells per well can be cultured in a 96-well plate. Target cell lines that express the TAA (TAA-expressing cells) or control cells (CHO-K1) can be seeded at a density of 2.5×10⁴ (hence an E:T ratio of 4:1, or a CD8⁺:T ratio of 1:1). The test antibodies can be serially diluted and added to a 96-well plate, at a final concentration starting from 100 nM. Following a 48 hours incubation at 37° C., the cells can be collected. After washing by PBS, the samples can be stained using standard procedures by incubation in the dark at room temperature for 30 minutes with the following antibodies: anti-human CD4-APC (Ebiosciene, 17-0048-42), anti-human CD8-BV510 (BD bioscience, 563919), anti-human 4-1BB-PE (BD Pharmingen, 555956). Samples can be washed twice with FACS buffer. After centrifugation, the supernatant can be discarded, and the human PBMC can be resuspended in 0.2 mL FACS buffer. The 4-1BB⁺ CD8⁺ T cell subsets amongst CD8⁺ T cells can be evaluated by MACSQuant Analyzer 16. The 4-1BB induction rates can be calculated as follows:

$$TAA - free 4 - 1 BB Induction Rate ( \% ) = \frac{\# of 4 - 1 {BB}^{+} CD {8}^{+} cells}{\# of total CD {8}^{+}} \% ( when control cells area used ) ; and \\ TAA - dependent 4 - 1 BB Induction Rate ( \% ) = \frac{\# of 4 - 1 {BB}^{+} CD {8}^{+} cells}{\# of total CD {8}^{+}} \% ( when TAA - expressing cells are used ) .$$

More briefly, in some embodiments, the TAA-free 4-1BB induction rate is measured by incubating, in the absence of TAA-expressing cells, human peripheral blood CD8+ cells with the multispecific antibody at a final concentration of 100 nM at 37° C. for 48 hours, and measuring the percentage of 4-1BB CD8⁺ cells over the total number of CD8⁺ cells as the TAA-free 4-1BB induction rate. In some embodiments, the multispecific antibody has a TAA-dependent 4-1BB induction rate of at least 20% at 100 nM, wherein the TAA-dependent 4-1BB induction rate is measured by: incubating human peripheral blood CD8⁺ cells and TAA-expressing cells at a ratio of 1:1, along with the multispecific antibody at a final concentration of 100 nM, at 37° C. for 48 hours, and measuring the percentage of 4-1BB⁺ CD8⁺ cells over the total number of CD8+ cells as the TAA-dependent 4-1BB induction rate.

In some embodiments, the multispecific antibody has considerably higher 4-1BB induction activity in the presence of TAA-expressing cells than in the absence thereof. For instance, in some embodiments, the multispecific antibody has a 4-1BB induction rate that is at least 2-fold as compared to the TAA-free 4-1BB induction rate. In some embodiments, the multispecific antibody has a 4-1BB induction rate that is at least 1.5-fold, 2-fold, 3-fold, 4-fold, 5-fold, 6-fold, 7-fold, 8-fold, 9-fold, 10-fold, 11-fold, 12-fold, 13-fold, 14-fold, 15-fold, or 20-fold as compared to the TAA-free 4-1BB induction rate.

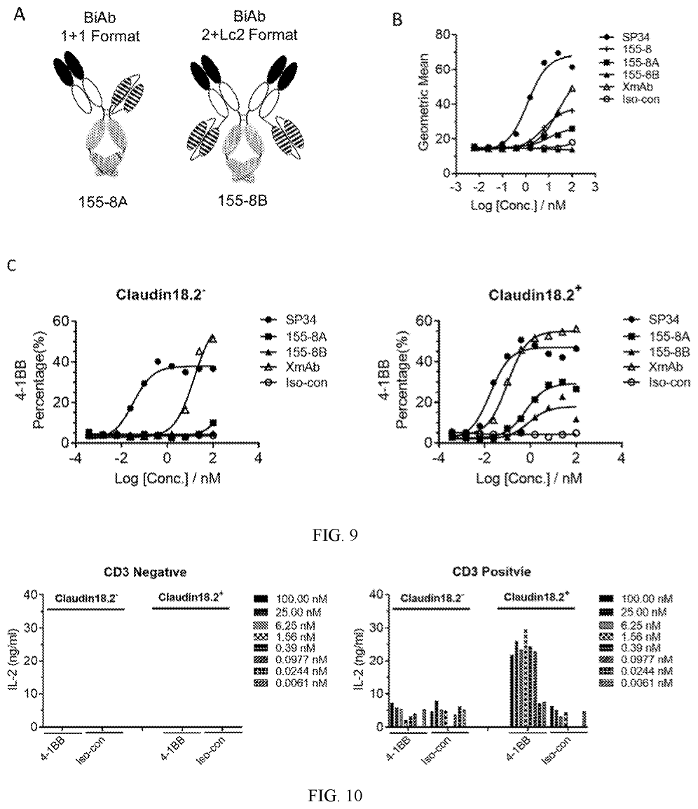

In some embodiments, the multispecific antibody is bispecific. In some embodiments, the multispecific antibody is trispecific. In some embodiments, the multispecific antibody has a trispecific format as shown in Format 1+1 (FIG. 9). In this format, the anti-TAA unit is a Fab fragment, the anti-CD3 unit is a scFv, and the anti-4-1BB unit includes two nanobodies fused to the C-terminus of the Fc fragment.

In some embodiments, the anti-CD3 unit in the multispecific antibody includes at least a single chain fragment (scFv). In some embodiments, the anti-CD3 unit in the multispecific antibody includes only one scFv. In some embodiments, the scFv is fused to the N-terminus of a Fc, optionally through a linker or hinge fragment.

In some embodiments, the anti-TAA unit in the multispecific antibody includes a Fab fragment consisting of a VH/VL pair. In some embodiments, the anti-TAA unit is fused to the N-terminus of a Fc, optionally through a linker or hinge fragment.

The Fc fragment of the multispecific antibody is optionally modified, as compared to wild-type human Fc fragments, to include knob-in-hole or modified salt bridges to reduce mispairing of the asymmetrical antibody.

In some embodiments, the multispecific antibody further includes an antigen-binding unit. In some embodiments, the additional antigen-binding unit has specificity to a human 4-1BB protein. In some embodiments, the anti-4-1BB unit is fused to the N-terminus of one or both of the two Fc chains. In some embodiments, the anti-4-1BB unit includes one or a pair of scFv. In some embodiments, the anti-4-1BB unit includes one or a pair of nanobodies. In some embodiments, the anti-4-1BB unit is a non-agonist anti-4-1BB antibody or fragment thereof. A non-agonist anti-4-1BB antibody or fragment binds to 4-1BB but does not activate it in the absence of binding to a tumor-associated antigen.

In some embodiments, an anti-CD3 antibody or fragment that is considered to have a balanced T cell activation activity has a TAA-free 4-1BB induction rate that is from 1% to 25%. In some embodiments, the 4-1BB induction rate is not greater than 25%, 24%, 23%, 22%, 21%, 20%, 9%1, 8%1, 7%1, 16%, 15%, 4%1, 3%1, 12%, 11%, 10%, 9%, 8%, 7%, 6%, 5%, 4%, 3% or 2%. In some embodiments, the 4-1BB induction rate is at least 1%, 2%, 3%, 4%, 5%, 6%, 7%, 8%, 9%, 10%, 110%, 12%, 13%, 14%, 15%, 20%, or 25%.

In some embodiments, the TAA-free 4-1BB induction rate for an anti-CD3 antibody or fragment that is considered to have a balanced T cell activation activity is from 1% to 25%, from 1% to 24%, from 1% to 23%, from 1% to 22%, from 1% to 21%, from 1% to 20%, from 1% to 19%, from 1% to 18%, from 1% to 17%, from 1% to 16%, from 1% to 15%, from 1% to 14%, from 1% to 13%, from 1% to 12%, from 1% to 11%, from 1% to 10%, from 2% to 25%, from 2% to 24%, from 2% to 23%, from 2% to 22%, from 2% to 21%, from 2% to 20%, from 2% to 19%, from 2% to 18%, from 2% to 17%, from 2% to 16%, from 2% to 15%, from 2% to 14%, from 2% to 13%, from 2% to 12%, from 2% to 11%, from 2% to 10%, from 3% to 25%, from 3% to 24%, from 3% to 23%, from 3% to 22%, from 3% to 21%, from 3% to 20%, from 3% to 19%, from 3% to 18%, from 3% to 17%, from 3% to 16%, from 3% to 15%, from 3% to 14%, from 3% to 13%, from 3% to 12%, from 3% to 11%, from 3% to 10%, from 4% to 25%, from 4% to 24%, from 4% to 23%, from 4% to 22%, from 4% to 21%, from 4% to 20%, from 4% to 19%, from 4% to 18%, from 4% to 17%, from 4% to 16%, from 4% to 15%, from 4% to 14%, from 4% to 13%, from 4% to 12%, from 4% to 11%, from 4% to 10%, from 5% to 25%, from 5% to 24%, from 5% to 23%, from 5% to 22%, from 5% to 21%, from 5% to 20%, from 5% to 19%, from 5% to 18%, from 5% to 17%, from 5% to 16%, from 5% to 15%, from 5% to 14%, from 5% to 13%, from 5% to 12%, from 5% to 11%, from 5% to 10%, from 6% to 25%, from 6% to 24%, from 6% to 23%, from 6% to 22%, from 6% to 21%, from 6% to 20%, from 6% to 19%, from 6% to 18%, from 6% to 17%, from 6% to 16%, from 6% to 15%, from 6% to 14%, from 6% to 13%, from 6% to 12%, from 6% to 11%, from 6% to 10%, from 7% to 25%, from 7% to 24%, from 7% to 23%, from 7% to 22%, from 7% to 21%, from 7% to 20%, from 7% to 19%, from 7% to 18%, from 7% to 17%, from 7% to 16%, from 7% to 15%, from 7% to 14%, from 7% to 13%, from 7% to 12%, from 7% to 11%, from 7% to 10%, from 8% to 25%, from 8% to 24%, from 8% to 23%, from 8% to 22%, from 8% to 21%, from 8% to 20%, from 8% to 19%, from 8% to 18%, from 8% to 17%, from 8% to 16%, from 8% to 15%, from 8% to 14%, from 8% to 13%, from 8% to 12%, from 8% to 11%, from 8% to 10%, from 9% to 25%, from 9% to 24%, from 9% to 23%, from 9% to 22%, from 9% to 21%, from 9% to 20%, from 9% to 19%, from 9% to 18%, from 9% to 17%, from 9% to 16%, from 9% to 15%, from 9% to 14%, from 9% to 13%, from 9% to 12%, from 9% to 11%, from 9% to 10%, from 10% to 25%, from 10% to 24%, from 10% to 23%, from 10% to 22%, from 10% to 21%, from 10% to 20%, from 10% to 19%, from 10% to 18%, from 10% to 17%, from 10% to 16%, from 10% to 15%, from 10% to 14%, from 10% to 13%, from 10% to 12%, or from 10% to 11%.

In some embodiments, for the measurement of the TAA-free 4-1BB induction rates, the final concentration of the antibody can be 100 nM, as tested herein, or alternatively 1 nM, 5 nM, 10 nM, 15 nM, 20 nM, 30 nM, 40 nM, or 50 nM, without limitation.

In some embodiments, an anti-CD3 antibody or fragment that is considered to have a balanced T cell activation activity has a TAA-dependent 4-1BB induction rate that is from 1% to 80%. In some embodiments, the TAA-dependent 4-1BB induction rate is not greater than 80%, 75%, 70%, 65%, 60%, 55%, 50%, 45%, 40%, 35%, 30%, 29%, 28%, 27%, 26%, or 25%. In some embodiments, the TAA-dependent 4-1BB induction rate is at least 15%, 20%, 25%, 30%, 35%, 40%, or 50%.

In some embodiments, the TAA-dependent 4-1BB induction rate for an anti-CD3 antibody or fragment that is considered to have a balanced T cell activation activity is from 15% to 80%, from 15% to 70%, from 15% to 60%, from 15% to 50%, from 15% to 40%, from 15% to 35%, from 20% to 80%, from 20% to 70%, from 20% to 60%, from 20% to 50%, from 20% to 40%, from 20% to 35%, from 20% to 30%, from 25% to 80%, from 25% to 70%, from 25% to 60%, from 25% to 50%, from 25% to 40%, from 25% to 35%, from 25% to 30%, from 30% to 80%, from 30% to 70%, from 30% to 60%, from 30% to 50%, from 30% to 40%, or from 30% to 35%.

In some embodiments, for the measurement of the TAA-dependent 4-1BB induction rates, the final concentration of the antibody can be 1 nM, as illustrated above, or alternatively 0.5 nM, 2 nM, 5 nM, 10 nM, 15 nM, 20 nM, 30 nM, 40 nM, 50 nM or 100 nM, without limitation. In one embodiment, the concentration is 5 nM. In one embodiment, the concentration is 10 nM. In one embodiment, the concentration is 20 nM.

In some embodiments, the multispecific antibody has a TAA-free 4-1BB induction rate of not greater than 5%, 10%, 15%, 20%, or 25% at an antibody concentration of 100 nM. In some embodiments, the multispecific antibody has a TAA-free 4-1BB induction rate at an antibody concentration of 100 nM of not greater than 15%, 14%, 14%, 13%, 12%, 11%, 10%, 9%, 8%, 7%, 6%, 5%, 4%, 3%, 2% or 1%. In some embodiments, the measurement is made at an alternative antibody concentration of 1 nM, 2 nM, 3 nM, 4 nM, 5 nM, 10 nM, 20 nM, 30 nM, 40 nM, 50 nM, 60 nM, 70 nM, 80 nM, 90 nm, 100 nM, 150 nM or 200 nM.

In some embodiments, the multispecific antibody has a TAA-dependent 4-1BB induction rate of at least 10% at 1 nM. In some embodiments, the multispecific antibody has a TAA-dependent 4-1BB induction rate, at an antibody concentration of 1 nM, of at least 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39% or 40%. In some embodiments, the measurement is made at an alternative antibody concentration of 1 nM, 2 nM, 3 nM, 4 nM, 5 nM, 10 nM, 20 nM, 30 nM, 40 nM, or 50 nM.

### T-Cell Engagers Synergizing CD3 and 4-1BB Agonism

Anti-CD3 agonist antibodies can activate 4-1BB induction. As provided above, the 4-1BB induction activity of the anti-CD3 antibody can be made “dependent” upon the presence of tumor antigen-expressing cells. That is, in the absence of the tumor antigen, the 4-1BB induction rate is low, and in the presence of the tumor antigen, the 4-1BB induction rate is many folds higher. Such tumor antigen dependency is beneficial to patients, as it reduces off-target toxicities.

In this context, it is further discovered herein that both the tumor antigen-dependency, and tumor antigen-dependent 4-1BB induction can be synergistically enhanced when a T cell engager includes both an anti-CD3 unit and an anti-4-1BB unit. In particular, when the anti-4-1BB unit itself is a tumor antigen-dependent agonistic antibody, the synergism is further increased. A tumor antigen-dependent agonistic anti-4-1BB antibody does not activate (or has low ability to activate) 4-1BB signaling without the presence of a tumor antigen, but activates 4-1BB signaling in the presence of a tumor antigen. Such an anti-4-1BB antibody is referred to as a “tumor antigen-dependent agonist anti-4-1BB antibody” or “non-agonist anti-4-1BB antibody.”

Interestingly, such synergism between anti-CD3 antibodies and anti-4-1BB antibodies has not been observed between anti-CD3 antibodies and antibodies targeting other T cell co-stimulatory factors, such as CD28. Therefore, this synergism is quite unexpected.

Another surprising discovery is that when both an anti-CD3 unit and an anti-4-1BB unit are included in a multispecific antibody, they are preferably located “away” from each other. For instance, in either the 1+1 format or the 2+Lc2 format as illustrated in FIG. 6, the anti-CD3 unit and the anti-4-1BB unit are preferably separated by an Fc fragment, rather than located at the same end of the Fc fragment. By contrast, when an anti-TAA unit is also included, the anti-CD3 unit and the anti-TAA unit are preferably located on the same side (e.g., N-terminal) of the Fc fragment.

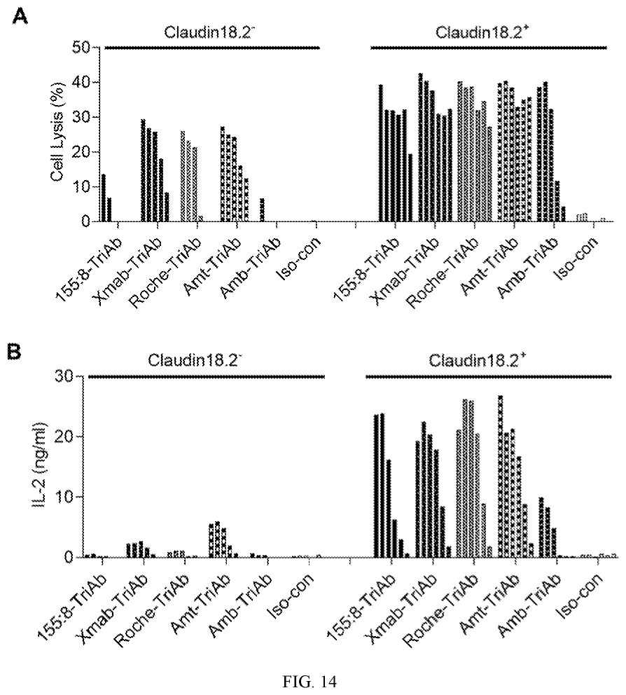

The dramatic efficacy of such multispecific antibodies is demonstrated in Example 18, with an in vivo animal model of B16F10. B16F10 is a well-known PD1 non-responsive and resistant model which is commonly referred to as a “cold tumor”. Cold tumors, by definition, are particularly difficult to treat. As expected, as shown in FIG. 14, the anti-PD1 therapy exhibited no efficacy. Bispecific antibodies (CTM01-O1A which did not target 4-1BB, and CTM01-01B which did not target CD3) that targeted only TAA and either CD3 or 4-1BB had suboptimal efficacies. The trispecific antibody (CTM01-01) that targeted all of TAA, CD3 and 4-1BB had far superior efficacy. This example, therefore, demonstrates the unexpectedly magnificent efficacy of targeting both CD3 and 4-1BB, in fashions as disclosed herein, in treating cold tumors.

Accordingly, one embodiment of the present disclosure provides a multispecific antibody that includes an anti-CD3 unit comprising an anti-CD3 antibody or antigen-binding fragment having binding specificity to the human CD3 complex; and an anti-4-1BB unit comprising an anti-4-1BB antibody or antigen-binding fragment having binding specificity to the human 4-1BB protein. In some embodiments, the multispecific antibody further includes an anti-tumor-associated antigen (TAA) unit comprising an anti-TAA antibody or antigen-binding fragment having binding specificity to a human TAA.

In some embodiments, the multispecific antibody further includes an Fc fragment. Example formats of multispecific antibodies with the Fc fragment are illustrated in FIG. 6. In some embodiments, the anti-CD3 unit and the anti-TAA unit are both located N-terminal to the Fc fragment.

In a 1+1 format, the anti-CD3 antibody or antigen-binding fragment is a single chain fragment (scFv) fused to the N-terminus of a chain of the Fc fragment, and the anti-TAA antibody or antigen-binding fragment is a Fab fragment fused to another chain of the Fc fragment.

In a 2+Lc2 format, the anti-TAA unit comprises two anti-TAA Fab fused, through the respective heavy chain, to the N-terminus of the Fc fragment, and the anti-CD3 unit comprises two scFv each fused to the C-terminus of a light chain of the Fab of the anti-TAA unit.

In both illustrated formats, the anti-4-1BB unit can be fused to the C-terminus of the Fc fragment. In some embodiments, the anti-4-1BB unit includes two anti-4-1BB nanobodies or scFv.

In some embodiments, the anti-4-1BB antibody or antigen-binding fragment is a tumor-associated antigen-dependent agonist antibody or antigen-binding fragment. In some embodiments, the anti-4-1BB antibody or antigen-binding fragment does not cluster 4-1BB proteins on the cell surface. Such antibodies are known in the art, such as 1A10 from Abl Bio.

Human 4-1BB, as a tumor necrosis factor receptor (TNFR) superfamily (TNFRSF) member, contains four cysteine-rich domains (CRD) in the N-terminal extracellular region connected to a C-terminal cytoplasmic region that contains a TNF receptor-associated factor (TRAF)-binding motif to initiate subsequent signaling. The locations of these four CRDs, CRD1, CRD2, CRD3 and CRD4 are shown in Table A.

4-1BB signaling activation is the expected mechanism for agonist antibodies, such as utomilumab (PF-05082566) and urelumab (BMS-663513). Urelumab binds to CRD1 and utomilumab binds to CRD3. A preferred anti-4-1BB antibody or fragment for incorporation into the multispecific antibody of the present disclosure does not bind to CRD1. Instead, it binds to CRD2, CRD3, or CRD4, or their combinations. In some embodiments, such an anti-4-1BB antibody or antigen-binding fragment does not have agonist activities.

TABLE A
Amino acid sequence of human 4-1BB (SEQ ID NO: 77)

| | |
|---|---|
| 1 | MGNSCYNIVA TLLLVLNFER TRSLQDPCSN CPAGTFCDNN RNQICSPCPP NSFSSAGGQR |
| | CRD1 |
| 61 | TCDICRQCKG VFRTRKECSS TSNAECDCTP GFHCLGAGCS MCEQDCKQGQ ELTKKGCKDC |
| | CRD2 CRD3 |
| 121 | CFGTENDQKR GICRPWTNCS LDGKSVLVNG TKERDVVCGP SPADLSPGAS SVTPPAPARE |
| | CRD4 |
| 181 | PGHSPQIISF FLALTSTALL FLLFFLTLRF SVVKRGRKKL LYIFKQPFMR PVQTTQEEDG |
| 241 | CSCRFPEEEE GGCEL |

A “tumor-associated antigen” or “tumor antigen” is an antigenic substance produced in tumor cells, i.e., it triggers an immune response in the host. Tumor antigens are useful in identifying tumor cells and are potential candidates for use in cancer therapy. Normal proteins in the body are not antigenic. Certain proteins, however, are produced or overexpressed during tumorigenesis and thus appear “foreign” to the body. This may include normal proteins that are well sequestered from the immune system, proteins that are normally produced in extremely small quantities, proteins that are normally produced only in certain stages of development, or proteins whose structure is modified due to mutation.

An abundance of tumor antigens are known in the art and new tumor antigens can be readily identified by screening. Non-limiting examples of tumor antigens include Claudin 18.2, 5T4, GPC3, EGFR, Her2, EpCAM, CD20, CD30, CD33, CD47, CD52, CD133, CD73, CEA, gpA33, Mucins, TAG-72, CIX, PSMA, folate-binding protein, GD2, GD3, GM2, VEGF, VEGFR, Integrin, αVβ3, α5β1, ERBB2, ERBB3, MET, IGF1R, EPHA3, TRAILR1, TRAILR2, RANKL, FAP and Tenascin. In a particular embodiment, the TAA is Claudin 18.2. Example antibodies that target Claudin 18.2 are known in the art, including those disclosed in, e.g., PCT publications WO/2020/200196, WO/2021/058000, WO/2020/147321, and WO/2019/219089.

In some embodiments, the antibody or antigen binding fragment binds an antigen expressed on the surface of an immune cell. In some embodiments, the antibody or antigen binding fragment binds to a cluster of differentiation molecule selected from the group consisting of: CD1a, CD1b, CD1c, CD1d, CD2, CD3, CD4, CD5, CD6, CD7, CDS, CD9, CD 10, CD11A, CD11B, CD11C, CDw12, CD13, CD14, CD15, CD15s, CD16, CDwl7, CD18, CD19, CD20, CD21, CD22, CD23, CD24, CD25, CD26, CD27, CD28, CD29, CD3Q, CD31,CD32, CD33, CD34, CD35, CD36, CD37, CD38, CD39, CD40, CD41, CD42a, CD42b, CD42c, CD42d, CD43, CD44, CD45, CD45RO, CD45RA, CD45RB, CD46, CD47, CD48, CD49a, CD49b, CD49c, CD49d, CD49e, CD49f, CD50, CD51, CD52, CD53, CD54, CD55, CD56, CD57, CD58, CD59, CDw60, CD6I, CD62E, CD62L, CD62P, CD63, CD64, CD65, CD66a, CD66b, CD66c, CD66d, CD66e, CD66E CD68, CD69, CD70, CD71, CD72, CD73, CD74, CD75, CD76, CD79o, 0079b, CD80, CD81, CD82, CD83, CDw84, CD85, CD86, CD87, CD88, CD89, CD90, CD91, CDw92, CD93, CD94, CD95, CD96, CD97, CD98, CD99, CD 100, CDIGI, CD 102, CD103, CD104, CD105, CD106, CD107a, CD107b, CDw′108, CD109, CD114, CD115, CD116. CD117. CD118, CD119, CD120a, CD120b, CD121a, CDwl21b, CD122, CD123, CD124, CD125, CD126, CD127, CDwl28, CD129, CD130, CDwl31, CD132, CD134, CD135, CDw136, CDwl37, CD138, CD139, CD140a, CD140b, CD141, CD142, CD143, CD144, CD145, CD146, CD147, CD148, CD15G, CD151, CD152, CD153,CD 154, CD155, CD156, CD157, CD158a, CD158b, CD161, CD162, CD163, CD164, CD165, CD166, and CD182.

### Example Anti-CD3 Antibodies

The present disclosure, in some embodiments, also provides example anti-CD3 antibodies and fragments which can be used alone, or be selected for incorporation into T cell engagers of the present technology.

In accordance with one embodiment of the present disclosure is an antibody or antigen-binding fragment thereof having binding specificity to a human CD3 complex, comprising a heavy chain variable region (VH) comprising a VH CDR1, VH CDR2, and VH CDR3, and a light chain variable region (VL) comprising a VL CDR1, VL CDR2 and VL CDR3.

In one embodiment, the antibody is 153A6B1 or a derivative thereof, or a humanized version thereof. In some embodiments, the VH CDR1 includes the amino acid sequence of SEQ ID NO:7, the VH CDR2 includes the amino acid sequence of SEQ ID NO:8, the VH CDR3 includes the amino acid sequence of SEQ ID NO:9, the VL CDR1 includes the amino acid sequence of SEQ ID NO:10, the VL CDR2 includes the amino acid sequence of SEQ ID NO:11, and the VL CDR3 includes the amino acid sequence of SEQ ID NO:12.

It has been observed that the VH CDR2 (RIRYNGDTSYNSALKS, SEQ ID NO:8) includes a G55 residue and a S61 residue (Kabat numbering) which are at risk of post-translational modifications (PTM). Therefore, in some embodiments, for 153A6B1 or a humanized or derived version, a G55A substitution and/or a S61A substitution can be introduced to the VH to prevent PTM. With such changes, the alternative VH CDR2 can be RIRYNADTSYNSALKS (SEQ ID NO:64), RIRYNGDTSYNAALKS (SEQ ID NO:65), or RIRYNADTSYNAALKS (SEQ ID NO:66).

Likewise, the VL CDR3 (LQHGSGYT, SEQ ID NO:12) includes a S93A residue that is at risk of PTM. Therefore, in some embodiments, for 153A6B1 or a humanized or derived version, a S93A substitution can be introduced to the VL to prevent PTM. With such a change, the alternative VL CDR3 can be LQHGAGYT (SEQ ID NO:67).

In some embodiments, the VH includes an amino acid sequence selected from the group consisting of SEQ ID NO:1 and 23-28, in particular SEQ ID NO:23-28. In some embodiments, the VH incorporates a G55A substitution, a S61A substitution, or the combination thereof. In some embodiments, the VL includes an amino acid sequence selected from the group consisting of SEQ ID NO:2 and 30-32, in particular SEQ ID NO:30-32. In some embodiments, the VL incorporates a S93A substitution.

In some embodiments, the VH includes SEQ ID NO:23 and the VL includes SEQ ID NO:30. In some embodiments, the VH includes SEQ ID NO:23 and the VL includes SEQ ID NO:31. In some embodiments, the VH includes SEQ ID NO:23 and the VL includes SEQ ID NO:32. In some embodiments, the VH includes SEQ ID NO:24 and the VL includes SEQ ID NO:30. In some embodiments, the VH includes SEQ ID NO:24 and the VL includes SEQ ID NO:31. In some embodiments, the VH includes SEQ ID NO:24 and the VL includes SEQ ID NO:32. In some embodiments, the VH includes SEQ ID NO:25 and the VL includes SEQ ID NO:30. In some embodiments, the VH includes SEQ ID NO:25 and the VL includes SEQ ID NO:31. In some embodiments, the VH includes SEQ ID NO:25 and the VL includes SEQ ID NO:32. In some embodiments, the VH includes SEQ ID NO:26 and the VL includes SEQ ID NO:30. In some embodiments, the VH includes SEQ ID NO:26 and the VL includes SEQ ID NO:31. In some embodiments, the VH includes SEQ ID NO:26 and the VL includes SEQ ID NO:32. In some embodiments, the VH includes SEQ ID NO:27 and the VL includes SEQ ID NO:30. In some embodiments, the VH includes SEQ ID NO:27 and the VL includes SEQ ID NO:31. In some embodiments, the VH includes SEQ ID NO:27 and the VL includes SEQ ID NO:32. In some embodiments, the VH includes SEQ ID NO:28 and the VL includes SEQ ID NO:30. In some embodiments, the VH includes SEQ ID NO:28 and the VL includes SEQ ID NO:31. In some embodiments, the VH includes SEQ ID NO:28 and the VL includes SEQ ID NO:32. In some embodiments, the VH incorporates a G55A substitution, a S61A substitution, or the combination thereof. In some embodiments, the VL incorporates a S93A substitution.

Through sequence analysis and comparison to activity data, it is observed herein that certain back mutations play important roles in determining the activity of the antibody. It is contemplated that the back mutations 27F, 78V, 29L, and 30T in the VH strongly promote the T cell activation activity of the antibody, while back mutations 37V, 71R, 48M, 67L, and 93T in the VH help maintain the balance of the activity (Grade 3 or 4).

In one embodiments therefore, an anti-CD3 antibody or fragment of the instant disclosure includes the CDRs of antibody 153A6B1 or their de-risked version (as described above) and further includes at least one of the back mutations 37V, 71R, 48M, 67L, and 93T in the VH. In some embodiments, the anti-CD3 antibody or fragment includes at least 37V in the VH. In some embodiments, the anti-CD3 antibody or fragment includes at least 71R in the VH. In some embodiments, the anti-CD3 antibody or fragment includes at least 48M in the VH. In some embodiments, the anti-CD3 antibody or fragment includes at least 67L in the VH. In some embodiments, the anti-CD3 antibody or fragment includes at least 93T in the VH.

In some embodiments, the anti-CD3 antibody or fragment includes at least 37V and 71R, 37V and 48M, 37V and 67L, or 37V and 93T in the VH. In some embodiments, the anti-CD3 antibody or fragment includes at least 71R and 48M, 71R and 67L, or 71R and 93T in the VH. In some embodiments, the anti-CD3 antibody or fragment includes at least 48M and 67L, or 48M and 93T in the VH. In some embodiments, the anti-CD3 antibody or fragment includes at least 67L and 93T in the VH. In some embodiments, the anti-CD3 antibody or fragment includes at least 3 of 37V, 71R, 48M, 67L, and 93T in the VH. In some embodiments, the anti-CD3 antibody or fragment includes at least 4 of 37V, 71R, 48M, 67L, and 93T in the VH. In some embodiments, the anti-CD3 antibody or fragment includes all of 37V, 71R, 48M, 67L, and 93T in the VH.

In some embodiments, the anti-CD3 antibody or fragment does not include at least one of 27F, 78V, 29L, and 30T in the VH. In some embodiments, the anti-CD3 antibody or fragment does not include at least 27F and 78V, 27F and 29L, or 27F and 30T in the VH. In some embodiments, the anti-CD3 antibody or fragment does not include 78V, 29L and 30T, or 27F, 29L and 30T, or 27F, 78V and 30T, or 27F, 78V and 29L in the VH. In some embodiments, the anti-CD3 antibody or fragment does not include any of 27F, 78V, 29L, and 30T in the VH.

In some embodiments, an anti-CD3 antibody or fragment of the instant disclosure includes the CDRs of antibody 153A6B1 or their de-risked version (as described above) and further includes at least one of the back mutations 37V, 71R, 48M, 67L, and 93T (or 2, 3, 4 or more thereof as described above), and does not include at least one of 27F, 78V, 29L, and 30T (or 2, 3, or more thereof as described above), in the VH.

In some embodiment, the anti-CD3 antibody or fragment is of Potency Grade 4 as defined in Table 10 (e.g., 153-2, 3, 4). In some embodiments, the VH includes SEQ ID NO:24 and the VL includes SEQ ID NO:30. In some embodiments, the VH includes SEQ ID NO:25 and the VL includes SEQ ID NO:30. In some embodiments, the VH includes SEQ ID NO:26 and the VL includes SEQ ID NO:30. In some embodiments, the VH incorporates a G55A substitution, a S61A substitution, or the combination thereof. In some embodiments, the VL incorporates a S93A substitution.

In some embodiments, the VH has at least 85%, 90% or 95% sequence identity to SEQ ID NO: 24, includes the CDRs of SEQ ID NO: 24, and includes at least one of the back mutations 37V, 71R, 48M, 67L, and 93T (or 2, 3, 4 or more thereof as described above), and does not include at least one of 27F, 78V, 29L, and 30T (or 2, 3, or more thereof as described above), and the and VL has at least 85%, 90% or 95% sequence identity to SEQ ID NO:30 and includes the CDRs of SEQ ID NO:30.

In some embodiments, the VH has at least 85%, 90% or 95% sequence identity to SEQ ID NO: 25, includes the CDRs of SEQ ID NO: 25, and includes at least one of the back mutations 37V, 71R, 48M, 67L, and 93T (or 2, 3, 4 or more thereof as described above), and does not include at least one of 27F, 78V, 29L, and 30T (or 2, 3, or more thereof as described above), and the and VL has at least 85%, 90% or 95% sequence identity to SEQ ID NO:30 and includes the CDRs of SEQ ID NO:30.

In some embodiments, the VH has at least 85%, 90% or 95% sequence identity to SEQ ID NO: 26, includes the CDRs of SEQ ID NO: 26, and includes at least one of the back mutations 37V, 71R, 48M, 67L, and 93T (or 2, 3, 4 or more thereof as described above), and does not include at least one of 27F, 78V, 29L, and 30T (or 2, 3, or more thereof as described above), and the and VL has at least 85%, 90% or 95% sequence identity to SEQ ID NO:30 and includes the CDRs of SEQ ID NO:30.

In some embodiment, the anti-CD3 antibody or fragment is of Potency Grade 3 as defined in Table 10 (e.g., 153-7, 13). In some embodiments, the VH includes SEQ ID NO:23 and the VL includes SEQ ID NO:31. In some embodiments, the VH includes SEQ ID NO:23 and the VL includes SEQ ID NO:32. In some embodiments, the VH incorporates a G55A substitution, a S61A substitution, or the combination thereof. In some embodiments, the VL incorporates a S93A substitution.

In some embodiments, the VH has at least 85%, 90% or 95% sequence identity to SEQ ID NO: 23, includes the CDRs of SEQ ID NO: 23, and includes at least one of the back mutations 37V, 71R, 48M, 67L, and 93T (or 2, 3, 4 or more thereof as described above), and does not include at least one of 27F, 78V, 29L, and 30T (or 2, 3, or more thereof as described above), and the and VL has at least 85%, 90% or 95% sequence identity to SEQ ID NO:31 and includes the CDRs of SEQ ID NO:31.

In some embodiments, the VH has at least 85%, 90% or 95% sequence identity to SEQ ID NO: 23, includes the CDRs of SEQ ID NO: 23, and includes at least one of the back mutations 37V, 71R, 48M, 67L, and 93T (or 2, 3, 4 or more thereof as described above), and does not include at least one of 27F, 78V, 29L, and 30T (or 2, 3, or more thereof as described above), and the and VL has at least 85%, 90% or 95% sequence identity to SEQ ID NO:32 and includes the CDRs of SEQ ID NO:32.

In one embodiment, the antibody is 155A9B1 or a derivative thereof, or a humanized version thereof. In some embodiments, the VH CDR1 includes the amino acid sequence of SEQ ID NO:13, the VH CDR2 includes the amino acid sequence of SEQ ID NO:14, the VH CDR3 includes the amino acid sequence of SEQ ID NO:15, the VL CDR1 includes the amino acid sequence of SEQ ID NO:16, the VL CDR2 includes the amino acid sequence of SEQ ID NO:11, and the VL CDR3 includes the amino acid sequence of SEQ ID NO:17.

It has been observed that the VH CDR2 (RVRYNGDTSYNSALKS, SEQ ID NO:14) includes a G55 residue and a S61 residue (Kabat numbering) which are at risk of post-translational modifications (PTM). Therefore, in some embodiments, for 155A9B1 or a humanized or derived version, a G55A substitution and/or a S61A substitution can be introduced to the VH to prevent PTM. With such changes, the alternative VH CDR2 can be RVRYNADTSYNSALKS (SEQ ID NO:68), RVRYNGDTSYNAALKS (SEQ ID NO:69), or RVRYNADTSYNAALKS (SEQ ID NO:70).

Likewise, the VL CDR3 (LQHNSGYT, SEQ ID NO:17) includes a S93A residue that is at risk of PTM. Therefore, in some embodiments, for 155A9B1 or a humanized or derived version, a S93A substitution can be introduced to the VL to prevent PTM. With such a change, the alternative VL CDR3 can be LQHNAGYT (SEQ ID NO:71).

In some embodiments, the VH includes an amino acid sequence selected from the group consisting of SEQ ID NO:3 and 34-41, in particular SEQ ID NO:3 and 36-41, or just SEQ ID NO:36-41. In some embodiments, the VH incorporates a G55A substitution, a S61A substitution, or the combination thereof. In some embodiments, the VL includes an amino acid sequence selected from the group consisting of SEQ ID NO:4 and 43-46, in particular SEQ ID NO:44-46. In some embodiments, the VL incorporates a S93A substitution.

In some embodiments, the VH includes SEQ ID NO:36 and the VL includes SEQ ID NO:44. In some embodiments, the VH includes SEQ ID NO:36 and the VL includes SEQ ID NO:45. In some embodiments, the VH includes SEQ ID NO:36 and the VL includes SEQ ID NO:46. In some embodiments, the VH includes SEQ ID NO:37 and the VL includes SEQ ID NO:44. In some embodiments, the VH includes SEQ ID NO:37 and the VL includes SEQ ID NO:45. In some embodiments, the VH includes SEQ ID NO:37 and the VL includes SEQ ID NO:46. In some embodiments, the VH includes SEQ ID NO:38 and the VL includes SEQ ID NO:44. In some embodiments, the VH includes SEQ ID NO:38 and the VL includes SEQ ID NO:45. In some embodiments, the VH includes SEQ ID NO:38 and the VL includes SEQ ID NO:46. In some embodiments, the VH includes SEQ ID NO:39 and the VL includes SEQ ID NO:44. In some embodiments, the VH includes SEQ ID NO:39 and the VL includes SEQ ID NO:45. In some embodiments, the VH includes SEQ ID NO:39 and the VL includes SEQ ID NO:46. In some embodiments, the VH includes SEQ ID NO:40 and the VL includes SEQ ID NO:44. In some embodiments, the VH includes SEQ ID NO:40 and the VL includes SEQ ID NO:45. In some embodiments, the VH includes SEQ ID NO:40 and the VL includes SEQ ID NO:46. In some embodiments, the VH includes SEQ ID NO:41 and the VL includes SEQ ID NO:44. In some embodiments, the VH includes SEQ ID NO:41 and the VL includes SEQ ID NO:45. In some embodiments, the VH includes SEQ ID NO:41 and the VL includes SEQ ID NO:46. In some embodiments, the VH incorporates a G55A substitution, a S61A substitution, or the combination thereof. In some embodiments, the VL incorporates a S93A substitution.

Through sequence analysis and comparison to activity data, it is observed herein that certain back mutations play important roles in determining the activity of the antibody. It is contemplated that the back mutations 27F, 78A, 29L, and 30T in the VH strongly promote the T cell activation activity of the antibody, while back mutations 37V, 71R, 48M, 67L, and 93T in the VH help maintain the balance of the activity (Grade 3 or 4). Further, back mutations 36F and 58I in the VL help maintain the balance of the activity (Grade 3 or 4).

In one embodiments therefore, an anti-CD3 antibody or fragment of the instant disclosure includes the CDRs of antibody 155A9B1 or their de-risked version (as described above) and further includes at least one of the back mutations 37V, 71R, 48M, 67L, and 93T in the VH. In some embodiments, the anti-CD3 antibody or fragment includes at least 37V in the VH. In some embodiments, the anti-CD3 antibody or fragment includes at least 71R in the VH. In some embodiments, the anti-CD3 antibody or fragment includes at least 48M in the VH. In some embodiments, the anti-CD3 antibody or fragment includes at least 67L in the VH. In some embodiments, the anti-CD3 antibody or fragment includes at least 93T in the VH.

In some embodiments, the anti-CD3 antibody or fragment includes at least 37V and 71R, 37V and 48M, 37V and 67L, or 37V and 93T in the VH. In some embodiments, the anti-CD3 antibody or fragment includes at least 71R and 48M, 71R and 67L, or 71R and 93T in the VH. In some embodiments, the anti-CD3 antibody or fragment includes at least 48M and 67L, or 48M and 93T in the VH. In some embodiments, the anti-CD3 antibody or fragment includes at least 67L and 93T in the VH. In some embodiments, the anti-CD3 antibody or fragment includes at least 3 of 37V, 71R, 48M, 67L, and 93T in the VH. In some embodiments, the anti-CD3 antibody or fragment includes at least 4 of 37V, 71R, 48M, 67L, and 93T in the VH. In some embodiments, the anti-CD3 antibody or fragment includes all of 37V, 71R, 48M, 67L, and 93T in the VH.

In some embodiments, the anti-CD3 antibody or fragment does not include at least one of 27F, 78A, 29L, and 30T in the VH. In some embodiments, the anti-CD3 antibody or fragment does not include at least 27F and 78A, 27F and 29L, or 27F and 30T in the VH. In some embodiments, the anti-CD3 antibody or fragment does not include 78A, 29L and 30T, or 27F, 29L and 30T, or 27F, 78A and 30T, or 27F, 78A and 29L in the VH. In some embodiments, the anti-CD3 antibody or fragment does not include any of 27F, 78A, 29L, and 30T in the VH.

In one embodiments therefore, an anti-CD3 antibody or fragment of the instant disclosure includes the CDRs of antibody 155A9B1 or their de-risked version (as described above) and further includes at least one of the back mutations 36F and/or 58I in the VL.

In some embodiments, an anti-CD3 antibody or fragment of the instant disclosure includes the CDRs of antibody 155A9B1 or their de-risked version (as described above) and further includes at least one of the back mutations 37V, 71R, 48M, 67L, and 93T (or 2, 3, 4 or more thereof as described above), and does not include at least one of 27F, 78A, 29L, and 30T (or 2, 3, or more thereof as described above), in the VH. In some embodiments, the VL includes at least one of the back mutations 36F and/or 58I.

In a particular embodiment, the VH includes SEQ ID NO:37 and the VL includes SEQ ID NO:45. In some embodiments, the VH includes SEQ ID NO:37 with a G55A substitution, a S61A substitution, or the combination thereof, and the VL includes SEQ ID NO:45. In some embodiments, the VH includes SEQ ID NO:37 and the VL includes SEQ ID NO:45 with a S93A substitution. In some embodiments, the VH includes SEQ ID NO:37 with a G55A substitution, a S61A substitution, or the combination thereof, and the VL includes SEQ ID NO:45 with a S93A substitution.

In some embodiment, the anti-CD3 antibody or fragment is of Potency Grade 4 as defined in Table 10 (e.g., 155-8, 9, 10, 14, 15, 16). In some embodiments, the VH includes SEQ ID NO:37 and the VL includes SEQ ID NO:45. In some embodiments, the VH includes SEQ ID NO:38 and the VL includes SEQ ID NO:45. In some embodiments, the VH includes SEQ ID NO:39 and the VL includes SEQ ID NO:45. In some embodiments, the VH includes SEQ ID NO:37 and the VL includes SEQ ID NO:46. In some embodiments, the VH includes SEQ ID NO:38 and the VL includes SEQ ID NO:46. In some embodiments, the VH includes SEQ ID NO:39 and the VL includes SEQ ID NO:46. In some embodiments, the VH incorporates a G55A substitution, a S61A substitution, or the combination thereof. In some embodiments, the VL incorporates a S93A substitution.

In some embodiments, the VH has at least 85%, 90% or 95% sequence identity to SEQ ID NO: 37, includes the CDRs of SEQ ID NO: 37, and includes at least one of the back mutations 37V, 71R, 48M, 67L, and 93T (or 2, 3, 4 or more thereof as described above), and does not include at least one of 27F, 78A, 29L, and 30T (or 2, 3, or more thereof as described above), and the and VL has at least 85%, 90% or 95% sequence identity to SEQ ID NO:45, includes the CDRs of SEQ ID NO:45 and includes at least back mutations 36F and/or 58I.

In some embodiments, the VH has at least 85%, 90% or 95% sequence identity to SEQ ID NO: 38, includes the CDRs of SEQ ID NO: 38, and includes at least one of the back mutations 37V, 71R, 48M, 67L, and 93T (or 2, 3, 4 or more thereof as described above), and does not include at least one of 27F, 78A, 29L, and 30T (or 2, 3, or more thereof as described above), and the and VL has at least 85%, 90% or 95% sequence identity to SEQ ID NO:45, includes the CDRs of SEQ ID NO:45 and includes at least back mutations 36F and/or 58I.

In some embodiments, the VH has at least 85%, 90% or 95% sequence identity to SEQ ID NO: 39, includes the CDRs of SEQ ID NO: 39, and includes at least one of the back mutations 37V, 71R, 48M, 67L, and 93T (or 2, 3, 4 or more thereof as described above), and does not include at least one of 27F, 78A, 29L, and 30T (or 2, 3, or more thereof as described above), and the and VL has at least 85%, 90% or 95% sequence identity to SEQ ID NO:45, includes the CDRs of SEQ ID NO:45 and includes at least back mutations 36F and/or 58I.

In some embodiments, the VH has at least 85%, 90% or 95% sequence identity to SEQ ID NO: 37, includes the CDRs of SEQ ID NO: 37, and includes at least one of the back mutations 37V, 71R, 48M, 67L, and 93T (or 2, 3, 4 or more thereof as described above), and does not include at least one of 27F, 78A, 29L, and 30T (or 2, 3, or more thereof as described above), and the and VL has at least 85%, 90% or 95% sequence identity to SEQ ID NO:46, includes the CDRs of SEQ ID NO:46 and includes at least back mutations 36F and/or 58I.

In some embodiments, the VH has at least 85%, 90% or 95% sequence identity to SEQ ID NO: 38, includes the CDRs of SEQ ID NO: 38, and includes at least one of the back mutations 37V, 71R, 48M, 67L, and 93T (or 2, 3, 4 or more thereof as described above), and does not include at least one of 27F, 78A, 29L, and 30T (or 2, 3, or more thereof as described above), and the and VL has at least 85%, 90% or 95% sequence identity to SEQ ID NO:46, includes the CDRs of SEQ ID NO:46 and includes at least back mutations 36F and/or 58I.

In some embodiments, the VH has at least 85%, 90% or 95% sequence identity to SEQ ID NO: 39, includes the CDRs of SEQ ID NO: 39, and includes at least one of the back mutations 37V, 71R, 48M, 67L, and 93T (or 2, 3, 4 or more thereof as described above), and does not include at least one of 27F, 78A, 29L, and 30T (or 2, 3, or more thereof as described above), and the and VL has at least 85%, 90% or 95% sequence identity to SEQ ID NO:46, includes the CDRs of SEQ ID NO:46 and includes at least back mutations 36F and/or 58I.

In some embodiment, the anti-CD3 antibody or fragment is of Potency Grade 3 as defined in Table 10 (e.g., 155-2, 3, 4). In some embodiments, the VH includes SEQ ID NO:37 and the VL includes SEQ ID NO:44. In some embodiments, the VH includes SEQ ID NO:38 and the VL includes SEQ ID NO:44. In some embodiments, the VH includes SEQ ID NO:39 and the VL includes SEQ ID NO:44. In some embodiments, the VH incorporates a G55A substitution, a S61A substitution, or the combination thereof. In some embodiments, the VL incorporates a S93A substitution.

In some embodiments, the VH has at least 85%, 90% or 95% sequence identity to SEQ ID NO: 37, includes the CDRs of SEQ ID NO: 37, and includes at least one of the back mutations 37V, 71R, 48M, 67L, and 93T (or 2, 3, 4 or more thereof as described above), and does not include at least one of 27F, 78A, 29L, and 30T (or 2, 3, or more thereof as described above), and the and VL has at least 85%, 90% or 95% sequence identity to SEQ ID NO:44, includes the CDRs of SEQ ID NO:44 and includes at least back mutations 36F and/or 58I.

In some embodiments, the VH has at least 85%, 90% or 95% sequence identity to SEQ ID NO: 38, includes the CDRs of SEQ ID NO: 38, and includes at least one of the back mutations 37V, 71R, 48M, 67L, and 93T (or 2, 3, 4 or more thereof as described above), and does not include at least one of 27F, 78A, 29L, and 30T (or 2, 3, or more thereof as described above), and the and VL has at least 85%, 90% or 95% sequence identity to SEQ ID NO:44, includes the CDRs of SEQ ID NO:44 and includes at least back mutations 36F and/or 58I.

In some embodiments, the VH has at least 85%, 90% or 95% sequence identity to SEQ ID NO: 39, includes the CDRs of SEQ ID NO: 39, and includes at least one of the back mutations 37V, 71R, 48M, 67L, and 93T (or 2, 3, 4 or more thereof as described above), and does not include at least one of 27F, 78A, 29L, and 30T (or 2, 3, or more thereof as described above), and the and VL has at least 85%, 90% or 95% sequence identity to SEQ ID NO:44, includes the CDRs of SEQ ID NO:44 and includes at least back mutations 36F and/or 58I.

In one embodiment, the antibody is 192A7B9 or a derivative thereof, or a humanized version thereof. In some embodiments, the VH CDR1 includes the amino acid sequence of SEQ ID NO:13, the VH CDR2 includes the amino acid sequence of SEQ ID NO:18, the VH CDR3 includes the amino acid sequence of SEQ ID NO:19, the VL CDR1 includes the amino acid sequence of SEQ ID NO:20, the VL CDR2 includes the amino acid sequence of SEQ ID NO:21, and the VL CDR3 includes the amino acid sequence of SEQ ID NO:22.

It has been observed that the VH CDR2 (RMRYNGDTSYNSALKS, SEQ ID NO:18) includes a G55 residue and a S61 residue (Kabat numbering) which are at risk of post-translational modifications (PTM). Therefore, in some embodiments, for 192A7B9 or a humanized or derived version, a G55A substitution and/or a S61A substitution can be introduced to the VH to prevent PTM. With such changes, the alternative VH CDR2 can be RMRYNADTSYNSALKS (SEQ ID NO:72), RMRYNGDTSYNAALKS (SEQ ID NO:73), or RMRYNADTSYNAALKS (SEQ ID NO:74).

Likewise, the VL CDR2 (IANSLQT, SEQ ID NO:21) includes a S53 residue that is at risk of PTM. Therefore, in some embodiments, for 192A7B9 or a humanized or derived version, a S53A substitution can be introduced to the VL to prevent PTM. With such a change, the alternative VL CDR2 can be IANALQT (SEQ ID NO:75).

Also, the VL CDR3 (LQHNSWYT, SEQ ID NO:22) includes a S93A residue that is at risk of PTM. Therefore, in some embodiments, for 192A7B9 or a humanized or derived version, a S93A substitution can be introduced to the VL to prevent PTM. With such a change, the alternative VL CDR3 can be LQHNAWYT (SEQ ID NO:76).

In some embodiments, the VH includes an amino acid sequence selected from the group consisting of SEQ ID NO:5 and 48-55, in particular SEQ ID NO:5 and 50-55, or just SEQ ID NO:50-55. In some embodiments, the VH incorporates a G55A substitution, a S61A substitution, or the combination thereof. In some embodiments, the VL includes an amino acid sequence selected from the group consisting of SEQ ID NO:6 and 57-62, in particular SEQ ID NO:59-62. In some embodiments, the VL incorporates a S53A substitution. In some embodiments, the VL incorporates a S93A substitution.

In some embodiments, the VH includes SEQ ID NO:50 and the VL includes SEQ ID NO:59. In some embodiments, the VH includes SEQ ID NO:50 and the VL includes SEQ ID NO:60. In some embodiments, the VH includes SEQ ID NO:50 and the VL includes SEQ ID NO:61. In some embodiments, the VH includes SEQ ID NO:50 and the VL includes SEQ ID NO:62. In some embodiments, the VH includes SEQ ID NO:51 and the VL includes SEQ ID NO:59. In some embodiments, the VH includes SEQ ID NO:51 and the VL includes SEQ ID NO:60. In some embodiments, the VH includes SEQ ID NO:51 and the VL includes SEQ ID NO:61. In some embodiments, the VH includes SEQ ID NO:51 and the VL includes SEQ ID NO:62. In some embodiments, the VH includes SEQ ID NO:52 and the VL includes SEQ ID NO:59. In some embodiments, the VH includes SEQ ID NO:52 and the VL includes SEQ ID NO:60. In some embodiments, the VH includes SEQ ID NO:52 and the VL includes SEQ ID NO:61. In some embodiments, the VH includes SEQ ID NO:52 and the VL includes SEQ ID NO:62. In some embodiments, the VH includes SEQ ID NO:53 and the VL includes SEQ ID NO:59. In some embodiments, the VH includes SEQ ID NO:53 and the VL includes SEQ ID NO:60. In some embodiments, the VH includes SEQ ID NO:53 and the VL includes SEQ ID NO:61. In some embodiments, the VH includes SEQ ID NO:53 and the VL includes SEQ ID NO:62. In some embodiments, the VH includes SEQ ID NO:54 and the VL includes SEQ ID NO:59. In some embodiments, the VH includes SEQ ID NO:54 and the VL includes SEQ ID NO:60. In some embodiments, the VH includes SEQ ID NO:54 and the VL includes SEQ ID NO:61. In some embodiments, the VH includes SEQ ID NO:54 and the VL includes SEQ ID NO:62. In some embodiments, the VH includes SEQ ID NO:55 and the VL includes SEQ ID NO:59. In some embodiments, the VH includes SEQ ID NO:55 and the VL includes SEQ ID NO:60. In some embodiments, the VH includes SEQ ID NO:55 and the VL includes SEQ ID NO:61. In some embodiments, the VH includes SEQ ID NO:55 and the VL includes SEQ ID NO:62. In some embodiments, the VH incorporates a G55A substitution, a S61A substitution, or the combination thereof. In some embodiments, the VL incorporates a S53A substitution. In some embodiments, the VL incorporates a S93A substitution.

In some embodiment, the anti-CD3 antibody or fragment is of Potency Grade 4 as defined in Table 10 (e.g., 192-7, 13). In some embodiments, the VH includes SEQ ID NO:50 and the VL includes SEQ ID NO:60. In some embodiments, the VH includes SEQ ID NO:50 and the VL includes SEQ ID NO:61.

In some embodiment, the anti-CD3 antibody or fragment is of Potency Grade 3 as defined in Table 10 (e.g., 192-4). In some embodiments, the VH includes SEQ ID NO:53 and the VL includes SEQ ID NO:59.

In some embodiments, the antibody or fragment is of class IgG1, IgG2, IgG3 or IgG4. In some embodiments, the antibody or fragment is antibody-dependent cellular cytotoxicity (ADCC)-competent. In some embodiments, the antibody or fragment is not ADCC-competent.

It will also be understood by one of ordinary skill in the art that antibodies as disclosed herein may be modified such that they vary in amino acid sequence from the naturally occurring binding polypeptide from which they were derived. For example, a polypeptide or amino acid sequence derived from a designated protein may be similar, e.g., have a certain percent identity to the starting sequence, e.g., it may be 60%, 70%, 75%, 80%, 85%, 90%, 95%, 98%, or 99% identical to the starting sequence.

In certain embodiments, the antibody comprises an amino acid sequence or one or more moieties not normally associated with an antibody. Exemplary modifications are described in more detail below. For example, an antibody of the disclosure may comprise a flexible linker sequence, or may be modified to add a functional moiety (e.g., PEG, a drug, a toxin, or a label).

Antibodies, variants, or derivatives thereof of the disclosure include derivatives that are modified, i.e., by the covalent attachment of any type of molecule to the antibody such that covalent attachment does not prevent the antibody from binding to the epitope. For example, but not by way of limitation, the antibodies can be modified, e.g., by glycosylation, acetylation, pegylation, phosphorylation, phosphorylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the antibodies may contain one or more non-classical amino acids.

In some embodiments, the antibodies may be conjugated to therapeutic agents, prodrugs, peptides, proteins, enzymes, viruses, lipids, biological response modifiers, pharmaceutical agents, or PEG.

The antibodies may be conjugated or fused to a therapeutic agent, which may include detectable labels such as radioactive labels, an immunomodulator, a hormone, an enzyme, an oligonucleotide, a photoactive therapeutic or diagnostic agent, a cytotoxic agent, which may be a drug or a toxin, an ultrasound enhancing agent, a non-radioactive label, a combination thereof and other such agents known in the art.

The antibodies can be detectably labeled by coupling it to a chemiluminescent compound. The presence of the chemiluminescent-tagged antigen-binding polypeptide is then determined by detecting the presence of luminescence that arises during the course of a chemical reaction. Examples of particularly useful chemiluminescent labeling compounds are luminol, isoluminol, theromatic acridinium ester, imidazole, acridinium salt and oxalate ester.

The antibodies can also be detectably labeled using fluorescence emitting metals such as ¹²Eu, or others of the lanthanide series. These metals can be attached to the antibody using such metal chelating groups as diethylenetriaminepentacetic acid (DTPA) or ethylenediaminetetraacetic acid (EDTA). Techniques for conjugating various moieties to an antibody are well known, see, e.g., Amon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. (1985); Hellstrom et al., “Antibodies For Drug Delivery”, in Controlled Drug Delivery (2nd Ed.), Robinson et al., (eds.), Marcel Dekker, Inc., pp. 623-53 (1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), Academic Press pp. 303-16 (1985), and Thorpe et al., “The Preparation And Cytotoxic Properties Of Antibody-Toxin Conjugates”, Immunol. Rev. (52:119-58 (1982)).

### Example T Cell Engager Formats and Sequences

Certain embodiments of the present disclosure provide a multispecific antibody, comprising: an anti-CD3 unit including an anti-CD3 antibody or antigen-binding fragment having binding specificity to the human CD3 complex; and an anti-4-1BB unit including an anti-4-1BB antibody or antigen-binding fragment having binding specificity to the human 4-1BB protein.

In some embodiments, the multispecific antibody further includes an anti-tumor-associated antigen (TAA) unit including an anti-TAA antibody or antigen-binding fragment having binding specificity to a human TAA. In some embodiments, the multispecific antibody further includes an Fc fragment.

In some embodiments, the anti-CD3 unit is located at the N-terminal side of the Fc fragment. In some embodiments, the anti-CD3 unit is monovalent. In some embodiments, the anti-CD3 unit has a format of a scFv, Fab, or nanobody.

In some embodiments, the anti-4-1BB unit is fused to the N-terminus or C-terminus of the Fc fragment or C-terminus of the light chain. In some embodiments, the anti-4-1BB unit comprises one or two anti-4-1BB nanobodies or scFv.

In some embodiments, the anti-TAA unit is located at the N-terminal side of the Fc fragment. In some embodiments, the anti-TAA unit is located at the C-terminal side of the Fc fragment. In some embodiments, the anti-TAA unit is monovalent or bivalent. In some embodiments, the anti-TAA unit has a format of a scFv, Fab, or nanobody.

In some embodiments, the multispecific antibody further includes an Fc fragment, wherein the anti-CD3 and anti-TAA unit is located at the N-terminal of the Fc fragment. In some embodiments, the anti-4-1BB unit is fused to the C-terminus of the Fc fragment. In some embodiments, the anti-CD3 and anti-TAA unit is monovalent; the anti-4-1BB unit includes two anti-4-1BB nanobodies or scFv.

In some embodiments, the anti-CD3 antibody or antigen-binding fragment is a single chain fragment (scFv) or a Fab fragment or a nanobody and fused to the N-terminus of a chain of the Fc fragment. In some embodiments, the anti-TAA antibody or antigen-binding fragment is a Fab fragment, or scFv or nanobody, and fused to another chain of the Fc fragment. The anti-4-1BB antibody or antigen-binding fragment is a Fab fragment or a nanobody or scFv, fused to the C-terminus of the Fc fragment.

In some embodiments, the anti-CD3 antibody is a single chain fragment (scFv) fused to the N-terminus of a chain of the Fc fragment in monovalent format, and the anti-TAA antibody or antigen-binding fragment is a Fab fragment fused to another chain of the Fc fragment in monovalent format, and the anti-4-1BB unit is nanobody, fused to the C-terminus of Fe and in bivalent format.

### Polynucleotides Encoding the Antibodies and Methods of Preparing the Antibodies

The present disclosure also provides isolated polynucleotides or nucleic acid molecules encoding the antibodies, variants or derivatives thereof of the disclosure. The polynucleotides of the present disclosure may encode the entire heavy and light chain variable regions of the antigen-binding polypeptides, variants or derivatives thereof on the same polynucleotide molecule or on separate polynucleotide molecules. Additionally, the polynucleotides of the present disclosure may encode portions of the heavy and light chain variable regions of the antigen-binding polypeptides, variants or derivatives thereof on the same polynucleotide molecule or on separate polynucleotide molecules.

Methods of making antibodies are well known in the art and described herein. In certain embodiments, both the variable and constant regions of the antigen-binding polypeptides of the present disclosure are fully human. Fully human antibodies can be made using techniques described in the art and as described herein. For example, fully human antibodies against a specific antigen can be prepared by administering the antigen to a transgenic animal which has been modified to produce such antibodies in response to antigenic challenge, but whose endogenous loci have been disabled. Exemplary techniques that can be used to make such antibodies are described in U.S. Pat. Nos. 6,150,584; 6,458,592; 6,420,140 which are incorporated by reference in their entireties.

In certain embodiments, the prepared antibodies will not elicit a deleterious immune response in the animal to be treated, e.g., in a human. In one embodiment, antigen-binding polypeptides, variants, or derivatives thereof of the disclosure are modified to reduce their immunogenicity using art-recognized techniques. For example, antibodies can be humanized, primatized, deimmunized, or chimeric antibodies can be made. These types of antibodies are derived from a non-human antibody, typically a murine or primate antibody, that retains or substantially retains the antigen-binding properties of the parent antibody, but which is less immunogenic in humans. This may be achieved by various methods, including (a) grafting the entire non-human variable domains onto human constant regions to generate chimeric antibodies; (b) grafting at least a part of one or more of the non-human complementarity determining regions (CDRs) into a human framework and constant regions with or without retention of critical framework residues; or (c) transplanting the entire non-human variable domains, but “cloaking” them with a human-like section by replacement of surface residues. Such methods are disclosed in Morrison et al., Proc. Natl. Acad. Sci. USA 57:6851-6855 (1984); Morrison et al., Adv. Immunol. 44:65-92 (1988); Verhoeyen et al., Science 239:1534-1536 (1988); Padlan, Molec. Immun. 25:489-498 (1991); Padlan, Molec. Immun. 31:169-217 (1994), and U.S. Pat. Nos. 5,585,089, 5,693,761, 5,693,762, and 6,190,370, all of which are hereby incorporated by reference in their entirety.

De-immunization can also be used to decrease the immunogenicity of an antibody. As used herein, the term “de-immunization” includes alteration of an antibody to modify T-cell epitopes (see, e.g., International Application Publication Nos.: WO/9852976 A1 and WO/0034317 A2). For example, variable heavy chain and variable light chain sequences from the starting antibody are analyzed and a human T-cell epitope “map” from each V region showing the location of epitopes in relation to complementarity-determining regions (CDRs) and other key residues within the sequence is created. Individual T-cell epitopes from the T-cell epitope map are analyzed in order to identify alternative amino acid substitutions with a low risk of altering activity of the final antibody. A range of alternative variable heavy and variable light sequences are designed comprising combinations of amino acid substitutions and these sequences are subsequently incorporated into a range of binding polypeptides. Typically, between 12 and 24 variant antibodies are generated and tested for binding and/or function. Complete heavy and light chain genes comprising modified variable and human constant regions are then cloned into expression vectors and the subsequent plasmids introduced into cell lines for the production of whole antibody. The antibodies are then compared in appropriate biochemical and biological assays, and the optimal variant is identified.

The binding specificity of antigen-binding polypeptides of the present disclosure can be determined by in vitro assays such as immunoprecipitation, radioimmunoassay (RIA) or enzyme-linked immunoabsorbent assay (ELISA).

### Treatment of Tumors, Particularly Cold Tumors

As described herein, the antibodies, variants or derivatives of the present disclosure may be used in certain treatment and diagnostic methods.

The present disclosure is further directed to antibody-based therapies which involve administering the antibodies of the disclosure to a patient such as an animal, a mammal, and a human for treating one or more of the disorders or conditions described herein. Therapeutic compounds of the disclosure include, but are not limited to, antibodies of the disclosure (including variants and derivatives thereof as described herein) and nucleic acids or polynucleotides encoding antibodies of the disclosure (including variants and derivatives thereof as described herein).

The antibodies of the disclosure can also be used to treat or inhibit cancer. In some embodiments, a tumor antigen (e.g., Claudin 18.2) is overexpressed in tumor cells. Accordingly, in some embodiments, provided are methods for treating a cancer in a patient in need thereof. The method, in one embodiment, entails administering to the patient an effective amount of an antibody of the present disclosure. In some embodiments, at least one of the cancer cells (e.g., stromal cells) in the patient expresses, over-express, or is induced to express the tumor antigen. Induction of a gene expression, for instance, can be done by administration of a tumor vaccine or radiotherapy.

Tumors that can be suitably treated include those of bladder cancer, non-small cell lung cancer, renal cancer, breast cancer, urethral cancer, colorectal cancer, head and neck cancer, squamous cell cancer, Merkel cell carcinoma, gastrointestinal cancer, stomach cancer, esophageal cancer, ovarian cancer, renal cancer, and small cell lung cancer. Accordingly, the presently disclosed antibodies can be used for treating any one or more such cancers.

In some embodiments, the tumors being treated are those that are particularly challenging to treat with conventional immuno-oncological therapies, such as with antibodies targeting immune checkpoints (ICPs). Sometimes, such tumors are referred to as “cold tumors” or “nonimmunogenic tumors.” As explained above, the presently disclosed multispecific antibodies exhibited dramatic efficacy in an in vivo animal model of B16F10, a “cold tumor” (see, e.g., Example 18). In some embodiments, accordingly, the present disclosure provides methods and uses for treating cold tumors with multispecific antibodies disclosed herein.

In some embodiments, a nonimmunogenic tumor is one that is not infiltrated with T cells, or that is deficient in T cell filtration, in antigen presenting cells (APCs), or in T cell activation, or has deficit in T cell homing into the tumor bed. All of prostate cancer, pancreatic cancer, and leukemia are nonimmunogenic. The vast majority of breast cancer (95%), colorectal cancer (95%), gastric cancer (87%), head and neck cancer (84%), liver cancer (83%), esophageal cancer (86%), cervical cancer (87%), and thyroid cancer (87%) are also nonimmunogenic. In addition, 83% of lung cancer, 79% of bladder cancer, 77% of kidney cancer, 70% uterus cancer, and 66% melanoma are also nonimmunogenic.

Identification of nonimmunogenic, or cold tumors can also be made with measurements of type, density and location of immune cells within the tumors. For instance, Galon and Bruni (Nature Reviews Drug Discovery volume 18, pages 197-218 (2019)) describes a standardized scoring system, Immunoscore, based on the quantification of two lymphocyte populations (CD3 and CD8), e.g., in resected tissues, for guided stratification of hot and cold tumors. The Immunoscore ranges from Immunoscore 0 (I0, for low densities, such as absence of both cell types in both regions) to I4 (high immune cell densities in both locations). By classifying cancers according to their immune infiltration, the scoring system provides an immune-based classification of tumors, including a definition of “hot” (highly infiltrated, Immunoscore I4) and “cold” (non-infiltrated, Immunoscore I0) tumors.

In some embodiments, the tumor is resistant to a treatment with immune checkpoint inhibitors, such as PD-L1 inhibitors, PD-1 inhibitors, CTLA-4 inhibitors, or the combinations thereof. In some embodiments, the cancer is prostate cancer, pancreatic cancer, or leukemia. In some embodiments, the cancer is breast cancer, colorectal cancer, gastric cancer, head and neck cancer, liver cancer, esophageal cancer, cervical cancer, or thyroid cancer. In some embodiments, the cancer is lung cancer, bladder cancer, kidney cancer, uterus cancer, or melanoma.

Additional diseases or conditions associated with increased cell survival, that may be treated, prevented, diagnosed and/or prognosed with the antibodies or variants, or derivatives thereof of the disclosure include, but are not limited to, progression, and/or metastases of malignancies and related disorders such as leukemia (including acute leukemias (e.g., acute lymphocytic leukemia, acute myelocytic leukemia (including myeloblastic, promyelocytic, myelomonocytic, monocytic, and erythroleukemia)) and chronic leukemias (e.g., chronic myelocytic (granulocytic) leukemia and chronic lymphocytic leukemia)), polycythemia vera, lymphomas (e.g., Hodgkin's disease and non-Hodgkin's disease), multiple myeloma, Waldenstrom's macroglobulinemia, heavy chain disease, and solid tumors including, but not limited to, sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyo sarcoma, colon carcinoma, pancreatic cancer, breast cancer, thyroid cancer, endometrial cancer, melanoma, prostate cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilm's tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, menangioma, melanoma, neuroblastoma and retinoblastoma.

A specific dosage and treatment regimen for any particular patient will depend upon a variety of factors, including the particular antibodies, variant or derivative thereof used, the patient's age, body weight, general health, sex, and diet, and the time of administration, rate of excretion, drug combination, and the severity of the particular disease being treated. Judgment of such factors by medical caregivers is within the ordinary skill in the art. The amount will also depend on the individual patient to be treated, the route of administration, the type of formulation, the characteristics of the compound used, the severity of the disease, and the desired effect. The amount used can be determined by pharmacological and pharmacokinetic principles well known in the art.

Methods of administration of the antibodies, variants or include but are not limited to intradermal, intramuscular, intraperitoneal, intravenous, subcutaneous, intranasal, epidural, and oral routes. The antigen-binding polypeptides or compositions may be administered by any convenient route, for example by infusion or bolus injection, by absorption through epithelial or mucocutaneous linings (e.g., oral mucosa, rectal and intestinal mucosa, etc.) and may be administered together with other biologically active agents. Thus, pharmaceutical compositions containing the antigen-binding polypeptides of the disclosure may be administered orally, rectally, parenterally, intracistemally, intravaginally, intraperitoneally, topically (as by powders, ointments, drops or transdermal patch), bucally, or as an oral or nasal spray.

The term “parenteral” as used herein refers to modes of administration which include intravenous, intramuscular, intraperitoneal, intrasternal, subcutaneous and intra-articular injection and infusion.

Administration can be systemic or local. In addition, it may be desirable to introduce the antibodies of the disclosure into the central nervous system by any suitable route, including intraventricular and intrathecal injection; intraventricular injection may be facilitated by an intraventricular catheter, for example, attached to a reservoir, such as an Ommaya reservoir. Pulmonary administration can also be employed, e.g., by use of an inhaler or nebulizer, and formulation with an aerosolizing agent.

It may be desirable to administer the antibodies polypeptides or compositions of the disclosure locally to the area in need of treatment; this may be achieved by, for example, and not by way of limitation, local infusion during surgery, topical application, e.g., in conjunction, with a wound dressing after surgery, by injection, by means of a catheter, by means of a suppository, or by means of an implant, said implant being of a porous, non-porous, or gelatinous material, including membranes, such as sialastic membranes, or fibers. Preferably, when administering a protein, including an antibody, of the disclosure, care must be taken to use materials to which the protein does not absorb.

### Compositions

The present disclosure also provides pharmaceutical compositions. Such compositions comprise an effective amount of an antibody, and an acceptable carrier.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. Further, a “pharmaceutically acceptable carrier” will generally be a non-toxic solid, semisolid or liquid filler, diluent, encapsulating material or formulation auxiliary of any type.

The term “carrier” refers to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents such as acetates, citrates or phosphates. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose are also envisioned. These compositions can take the form of solutions, suspensions, emulsion, tablets, pills, capsules, powders, sustained-release formulations and the like. The composition can be formulated as a suppository, with traditional binders and carriers such as triglycerides. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in Remington's Pharmaceutical Sciences by E. W. Martin, incorporated herein by reference. Such compositions will contain a therapeutically effective amount of the antigen-binding polypeptide, preferably in purified form, together with a suitable amount of carrier so as to provide the form for proper administration to the patient. The formulation should suit the mode of administration. The parental preparation can be enclosed in ampoules, disposable syringes or multiple dose vials made of glass or plastic.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

The compounds of the disclosure can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

### EXAMPLES

### Example 1: Generation and Testing of Anti-Human CD3 Antibodies

This example describes the generation of anti-human-CD3 monoclonal antibodies using the hybridoma technology.

Antigen: Human CD3D & CD3E Heterodimer Protein (Sino biological, CT026-H0323H).

Immunization: To generate monoclonal antibodies to human CD3, Wistar rats and SD rats were immunized with CD3D & CD3E heterodimer protein. Post 4 rounds of immunization, the serum of immunized rats was subject to the antibody titer evaluation by ELISA. Briefly, microtiter plates were coated with human CD3 protein at 0.5 or 1 μg/ml in ELISA coating buffer, 100 μl/well at 4° C. overnight, then blocked with 200 μl/well of 5% non-fat milk. Dilutions of serum from immunized mice were added to each well and incubated for 1-2 hours at 37° C. The plates were washed with PBS/Tween and then incubate with Peroxidase AffiniPure Goat Anti-Rat IgG for 30 min at 37° C. After washing, the plates were developed with TMB substrate and analyzed by spectrophotometer at OD 450 nm. Rats with sufficient titers of anti-CD3 IgG were boosted with human CD3D & CD3E heterodimer protein.

Cell fusion: Cell fusion was performed by electro fusion. Fused cells were plated into 50 96-well plates for each fusion.

Subcloning and screening: Positive primary clones from each fusion were subcloned by limiting dilutions to ensure that the subclones were derived from a single parental cell. Subcloning were screened in the same approach as primary clones and culture supernatant of positive clones underwent additional confirmative screening by affinity ranking.

Hybridoma clones 153A6B1, 155A9B1, and 192A7B9 were selected for further analysis. The amino acid sequences of the variable regions of 153A6B1, 155A9B1, and 192A7B9 are provided in Table 1 below, with the CDR sequences summarized in Table 1A.

TABLE 1
Antibody Variable Region Sequences

| | | SEQ |
|---|---|---|
| | | ID |
| Name | Sequence | NO: |
| 153A6B1 VH | QVQLKESGPGLVQPSQTLSLTCTVSGFSLTSYNIHWVRQPPGKGLEWMGR | 1 |
| | IRYNGDTSYNSALKSRLSISRDTSKNQVFLKMSSLQTDDTGTYYCTRGPY | |
| | GGSREPLFDYWGQGVMVTVSS | |
| 153A6B1 VL | DIKMTQSPSFLSAAVGDRVTINCKTSQNINKYLNWFQQKLGAAPKLLIYT | 2 |
| | TNNLQTGIPSRFSGSGSGTDFTLTISSLQPEDVATYFCLQHGSGYTFGAG | |
| | TKLELK | |
| 155A9B1 VH | QVQLKESGPGLVQPSQTLSLTCTVSGFSLTSYNVHWVRQPPGKGLEWMGR | 3 |
| | VRYNGDTSYNSALKSRLSISRDTSKNQAFLKMNSLQIDDTGTYYCTRGPY | |
| | GGSSEPLFDYWGQGVMVTVSS | |
| 155A9B1 VL | DIKMTQSPSFLSASVGDRVTINCKASQNINKYLNWFQQKLGEAPRLLIYT | 4 |
| | TNNLQTGIPSRFSGSGSGTDFTLTISSLQPEDVATYFCLQHNSGYTFGAG | |
| | TKLELK | |
| 192A7B9 VH | QVQLKESGPGLVQPSQTLSLNCTVSGFSLTSYNVHWVRQPPGKGLEWMGR | 5 |
| | MRYNGDTSYNSALKSRLSISRDTSKNQVFLKMNSLQTDDTGTYYCTRGPR | |
| | GGYYSALFDYWGQGVMVTVSS | |
| 192A7B9 VL | DIQMTQSPSFLSASVGDRVTINCKASQNINRYLNWYQQKLGDAPKVLISI | 6 |
| | ANSLQTGIPSRFSGSGSGTDFTLTISSLQPEDVATYFCLQHNSWYTFGAG | |
| | TKLELK | |

TABLE 1A
CDR Sequences (with optional mutations to
aV0id PTM, Kabat numbering)

| | | | SEQ |
|---|---|---|---|
| | | | ID |
| Name | CDR | Sequences | NO: |
| 153A6B1 VH | CDR1 | SYNIH | 7 |
| | CDR2 | RIRYNGDTSYNSALKS | 8 |
| | (with G55A) | RIRYNADTSYNSALKS | 64 |
| | (with S61A) | RIRYNGDTSYNAALKS | 65 |
| | (with G55A/S61A) | RIRYNADTSYNAALKS | 66 |
| | CDR3 | GPYGGSREPLFDY | 9 |
| 153A6B1 VL | CDR1 | KTSQNINKYLN | 10 |
| | CDR2 | TTNNLQT | 11 |
| | CDR3 | LQHGSGYT | 12 |
| | (with S93A) | LQHGAGYT | 67 |
| 155A9B1 VH | CDR1 | SYNVH | 13 |
| | CDR2 | RVRYNGDTSYNSALKS | 14 |
| | (with G55A) | RVRYNADTSYNSALKS | 68 |
| | (with S61A) | RVRYNGDTSYNAALKS | 69 |
| | (with G55A/S61A) | RVRYNADTSYNAALKS | 70 |
| | CDR3 | GPYGGSSEPLFDY | 15 |
| 155A9B1 VL | CDR1 | KASQNINKYLN | 16 |
| | CDR2 | TTNNLQT | 11 |
| | CDR3 | LQHNSGYT | 17 |
| | (with S93A) | LQHNAGYT | 71 |
| 192A7B9 VH | CDR1 | SYNVH | 13 |
| | CDR2 | RMRYNGDTSYNSALKS | 18 |
| | (with G55A) | RMRYNADTSYNSALKS | 72 |
| | (with S61A) | RMRYNGDTSYNAALKS | 73 |
| | (with G55A/S61A) | RMRYNADTSYNAALKS | 74 |
| | CDR3 | GPRGGYYSALFDY | 19 |
| 192A7B9 VL | CDR1 | KASQNINRYLN | 20 |
| | CDR2 | IANSLQT | 21 |
| | (with S53A) | IANALQT | 75 |
| | CDR3 | LQHNSWYT | 22 |
| | (with S93A) | LQHNAWYT | 76 |

### Example 2: Binding Activity of Chimeric Antibodies to CD3 Antigen

### ELISA Testing

To evaluate the binding activity of hybridoma clones 153A6B 1, 155A9B 1, and 192A7B9, the chimeric mAb from these clones were subjected to ELISA test.

Briefly, microtiter plates were coated with human, cynomolgus and mouse CD3 protein at 1 μg/ml in PBS, 100 μl/well at 4° C. overnight, then blocked with 150 μl/well of 100 BSA. Three-fold dilutions of 153A6B1, 155A9B1, and 192A7B9 antibodies starting from 15 μg/ml were added to each well and incubated for 1 hour at 37° C. The plates were washed with PBS/Tween and then incubated with anti-Human IgG (H&L) (GOAT) Antibody Peroxidase Conjugated for 30 mins at 37° C. After washing, the plates were developed with TMB substrate and analyzed by spectrophotometer at OD 450 nm. As shown in Table 2, all of 153A6B1, 155A9B1, and 192A7B9 bound to both human and cynomolgus CD3. All the tested antibodies did not bind to mouse CD3. SP34 (Biointron, B6762) served as the benchmark controls. The sequences of SP34 are provided in Table 2B.

TABLE 2A
Cross species activity of
153A6B1, 155A9B1 and 192A7B9

| | Human | Cynomolgus | Mouse |
|---|---|---|---|
| | (ng/ml) | (ng/ml) | (ng/ml) |
| EC50 of 155A9B1 | 2.148 | 2.331 | NA |
| EC50 of 192A7B9 | 1.905 | 2.164 | NA |
| EC50 of 153A6B1 | 2.794 | 2.880 | NA |
| EC50 of SP34 | 6.484 | 6.177 | NA |

NA: Not Available

TABLE 2B
Sequences of SP34 monoclonal antibody

| | | SEQ |
|---|---|---|
| | | ID |
| Domain | Sequence | NO: |
| VH | EVQLVESGGGLVQPKGSLKLSCAASGFT | 78 |
| | FNTYAMNWVRQAPGKGLEWVARIRSKYN | |
| | NYATYYADSVKDRFTISRDDSQSILYLQ | |
| | MNNLKTEDTAMYYCVRHGNFGNSYVSWF | |
| | AYWGQGTLVTVSAASTKGPSVFPLAPSS | |
| | KSTSGGTAALGCLVKDYFPEPVTVSWNS | |
| | GALTSGVHTFPAVLQSSGLYSLSSVVTV | |
| | PSSSLGTQTYICNVNHKPSNTKVDKKVE | |
| | PKSCDKTHTCPPCPAPELLGGPSVFLFP | |
| | PKPKDTLMISRTPEVTCVVVDVSHEDPE | |
| | VKFNWYVDGVEVHNAKTKPREEQYASTY | |
| | RVVSVLTVLHQDWLNGKEYKCKVSNKAL | |
| | PAPIEKTISKAKGQPREPQVYTLPPSRE | |
| | EMTKNQVSLTCLVKGFYPSDIAVEWESN | |
| | GQPENNYKTTPPVLDSDGSFFLYSKLTV | |
| | DKSRWQQGNVFSCSVMHEALHNHYTQKS | |
| | LSLSPGK | |
| VL | QAVVTQESALTTSPGETVTLTCRSSTGA | 79 |
| | VTTSNYANWVQEKPDHLFTGLIGGTNKR | |
| | APGVPARFSGSLIGDKAALTITGAQTED | |
| | EAIYFCALWYSNLWVFGGGTKLTVLGQP | |
| | KANPTVTLFPPSSEELQANKATLVCLIS | |
| | DFYPGAVTVAWKADGSPVKAGVETTKPS | |
| | KQSNNKYAASSYLSLTPEQWKSHRSYSC | |
| | QVTHEGSTVEKTVAPTECS | |

### Cell-Based Binding

FACS was used to evaluate the binding activity of 153A6B1, 155A9B1, and 192A7B9 chimeric mAbs on human or cynomolgus PBMC. SP34 (Biointron, B6762) and OKT3 (Biointron, B6928) served as the benchmark controls.

Briefly, cynoPBMCs were first incubated with 3-fold serially diluted 153A6B1, 155A9B1, and 192A7B9 chimeric mAbs starting at 10 nM at 4° C. for 30 mins. While huPBMCs were incubated with 4-fold serially diluted 153A6B1, 155A9B1, and 192A7B9 chimeric mAbs starting at 10 nM at 4° C. for 30 mins. After washing by PBS, PE Goat anti-Human IgG Fc Secondary Antibody (eBioscience™, Invitrogen) was added to each well and incubated at 4° C. for 30 mins. Samples were washed twice with FACS buffer. The mean florescence intensity (MFI) of PE was evaluated by MACSQuant Analyzer 16. As shown in FIGS. 1A and 1B, 153A6B1, 155A9B1, and 192A7B9 bound to both human PBMC and cyno PBMC.

### Example 3: Binding Affinity of CD3 Chimeric Antibodies

The binding of the 153A6B1, 155A9B1, and 192A7B9 antibodies to recombinant CD3D&E protein (human CD3-his tag) was tested with Biacore using a capture method. The 153A6B1, 155A9B1, and 192A7B9 mAbs were captured using Protein A chip. A serial dilution of human CD3-his tag protein was injected over captured antibody for 3 mins at a flow rate of 30 μl/min. The antigen was allowed to dissociate for 120-360 s. All the experiments were carried out on a Biacore T200. Data analysis was carried out using Biacore T200 evaluation software. The results are shown in Table 3.

TABLE 3
Affinity measured by Biacore

| | CD3D&E-His | | |
|---|---|---|---|
| Abs | ka (1/Ms) | kd (1/s) | KD (M) |
| 155A9B1 | 5.719E+4 | 2.068E−3 | 3.615E−8 |
| 192A7B9 | 6.967E+4 | 4.416E−4 | 6.338E−9 |
| 153A6B1 | 7.986E+4 | 6.939E−4 | 8.690E−9 |
| SP34 | 4.237E+5 | 2.918E−3 | 6.888E−9 |
| OKT3 | 7.347E+5 | 2.719E−3 | 3.702E−9 |

### Example 4. Functional Activity of CD3 Chimeric Antibodies

This example tested the functional activities of the chimeric CD3 antibodies.

### Cell-Line Based Functional Characterization of CD3 Chimeric Antibodies

To assess the ability of the CD3 chimeric antibodies to activate the CD3 signaling pathway, a commercial CD3 NFAT luciferase reporter system was used. In this assay, Jurkat-CD3-NFAT was used as the reporter cell line. The Jurkat-CD3-NFAT cell line is genetically modified to stably express CD3 and luciferase downstream of a response element (Genomeditech #C17940). Luciferase expression is induced upon antibody binding to the CD3 receptor. Briefly, reporter cells at a density of 2.5×10⁴ cells per well were cultured in a white 96-well plate. Antibodies were 3-fold serially diluted and added to a white 96-well assay plate, at a final concentration ranging from 10 nM to 0.0005 nM. Following a 6 hours incubation at 37° C., luminescence was obtained by adding the substrate of luciferase and measured by a microplate reader. Four-parameter logistic curve analysis was performed with GraphPad software.

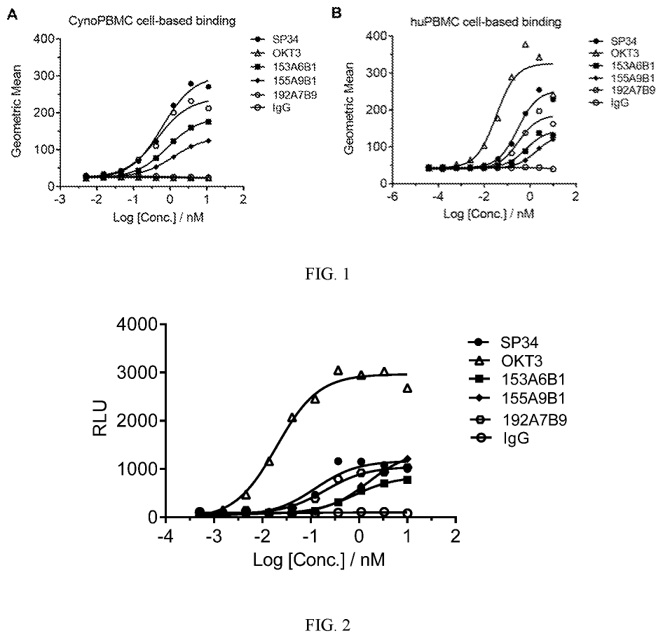

As shown in FIG. 2, all of 153A6B1, 155A9B1, and 192A7B9 antibodies displayed weaker CD3 NFAT activity as compared to OKT3, and 192A7B9 showed comparable CD3 NFAT activity to SP34.

### Example 5. 4-1BB Expression in CD8⁺ Cells Induced by CD3 Chimeric Antibodies

This example tested the 4-1BB induction in CD8⁺ cells triggered by the chimeric CD3 antibodies.

To assess the ability of the CD3 chimeric antibodies to induce 4-1BB expression, human PBMC at a density of 1×10⁵ cells per well were cultured in a 96-well plate. Chimeric antibodies were 3-fold serially diluted and added to a 96-well plate, at a final concentration ranging from 10 nM to 0.0015 nM. Following a 48 hours incubation at 37° C., human PBMCs were collected for further analysis. After washing by PBS, Samples were stained using standard procedures by incubation in the dark at room temperature for 30 minutes with the following antibodies: anti-human CD4-APC (Ebiosciene, 17-0048-42), anti-human CD8-BV510 (BD bioscience, 563919), anti-human CD137-PE (BD Pharmingen, 555956). Samples were washed twice with FACS buffer. After centrifugation, the supernatant was discarded, and the human PBMCs were resuspended in 0.2 mL FACS buffer. The CD137⁺ CD8⁺ T cell subsets amongst CD8⁺ T cells were evaluated by MACSQuant Analyzer 16.

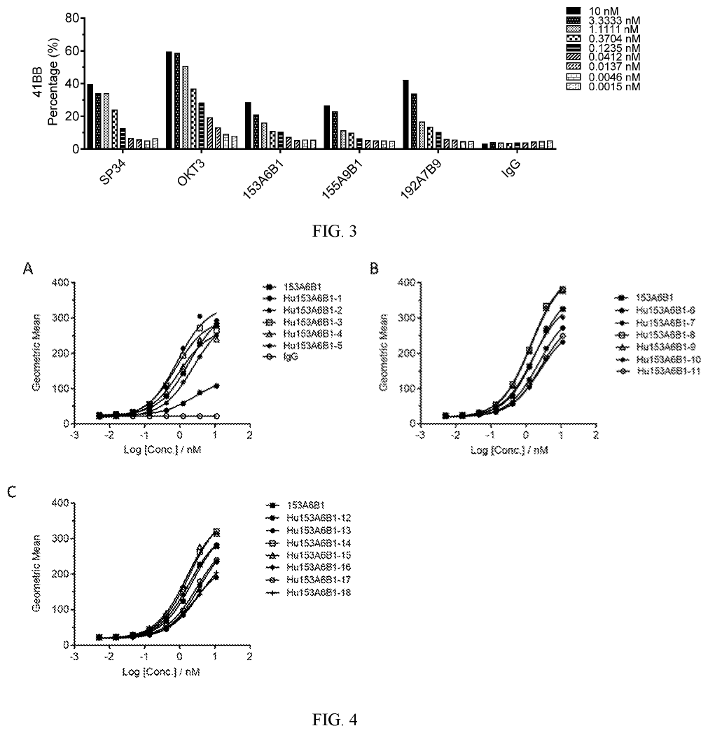

As shown in FIG. 3, like SP34 and OKT3, all three chimeric antibodies exhibited strong 4-1BB induction activities.

### Example 6. Humanization of the CD3 Antibodies

The 153A6B1/155A9B1/192A7B9 variable region genes were employed to create a humanized mAb. In the first step of this process, the amino acid sequences of the VH and VK of 153A6B1/155A9B1/192A7B9 were compared against the available database of human Ig gene sequences to find the overall best-matching human germline Ig gene sequences.

For the light chain of 153A6B1, VK1-39(O12)-JK2 is the best fit germline, and for the heavy chain of 153A6B1, VH4-59-JH3 was chosen as the humanization backbone. Humanized 153A6B1 CDR grafting antibody was then designed where the CDRL1, L2, and L3 were grafted onto framework sequences of the VK1-39(O12)-JK2, and the CDRH1, H2, and H3 were grafted onto framework sequences of the VH4-59-JH3. A 3D model was then generated to determine the amino acids in the original mouse FR region sequences that are essential for antibody binding and conformation. Based on the 153A6B1 CDR grafting antibody sequence, 6 additional humanized heavy chains and 3 additional light chains were created.

For the light chain of 155A9B1, VK1-39(O12)-JK2 is the best fit germline, and for the heavy chain of 155A9B1, VH4-59-JH3 was chosen as the humanization backbone. Humanized 155A9B1 CDR grafting antibody was then designed where the CDRL1, L2, and L3 were grafted onto framework sequences of the VK1-39(O12)-JK2, and the CDRH1, H2, and H3 were grafted onto framework sequences of the VH4-59-JH3. A 3D model was then generated to determine the amino acids in the original mouse FR region sequences that are essential for antibody binding and conformation. Based on the 155A9B1 CDR grafting antibody sequence, 6 additional humanized heavy chains and 3 additional light chains were created.

For the light chain of 192A7B9, VK1-39(O12)-JK2 is the best fit germline, and for the heavy chain of 192A7B9, VH4-59-JH3 was chosen as the humanization backbone. Humanized 192A7B9 CDR grafting antibody was then designed where the CDRL1, L2, and L3 were grafted onto framework sequences of the VK1-39(O12)-JK2, and the CDRH1, H2, and H3 were grafted onto framework sequences of the VH4-59-JH3. A 3D model was then generated to determine the amino acids in the original mouse FR region sequences that are essential for antibody binding and conformation. Based on the 192A7B9 CDR grafting antibody sequence, 6 additional humanized heavy chains and 4 additional light chains were created.

The sequences of the human germlines used for CDR grafting, as well as the resulting humanized sequences are listed in Tables 4-6.

TABLE 4
Humanization of 153A6B1

| | | SEQ |
|---|---|---|
| | | ID |
| Name | Sequence | NO: |
| 153A6B1 VH | QVQLKESGPGLVQPSQTLSLTCTVSGFSLTSYNIHWVRQPPGKGLEWMGR | 1 |
| | IRYNGDTSYNSALKSRLSISRDTSKNQVFLKMSSLQTDDTGTYYCTRGPY | |
| | GGSREPLFDYWGQGVMVTVSS | |
| 153A6B1 VH.V0 | EVQLQESGPGLVKPSETLSLTCTVSGGSISSYNIHWIRQPPGKGLEWIGR | 23 |
| (grafted) | IRYNGDTSYNSALKSRVTISVDTSKNQFSLKLSSVTAADTAVYYCARGPY | |
| | GGSREPLFDYWGQGTMVTVSS | |
| 153A6B1 VH.V1 | EVQLQESGPGLVKPSETLSLTCTVSGGSISSYNIHWVRQPPGKGLEWIGR | 24 |
| | IRYNGDTSYNSALKSRVTISRDTSKNQFSLKLSSVTAADTAVYYCARGPY | |
| | GGSREPLFDYWGQGTMVTVSS | |
| 153A6B1 VH.V2 | EVQLQESGPGLVKPSETLSLTCTVSGGSISSYNIHWVRQPPGKGLEWMGR | 25 |
| | IRYNGDTSYNSALKSRLTISRDTSKNQFSLKLSSVTAADTAVYYCARGPY | |
| | GGSREPLFDYWGQGTMVTVSS | |
| 153A6B1 VH.V3 | EVQLQESGPGLVKPSETLSLTCTVSGGSISSYNIHWVRQPPGKGLEWMGR | 26 |
| | IRYNGDTSYNSALKSRLTISRDTSKNQFSLKLSSVTAADTAVYYCTRGPY | |
| | GGSREPLFDYWGQGTMVTVSS | |
| 153A6B1 VH.V4 | EVQLQESGPGLVKPSETLSLTCTVSGFSISSYNIHWVRQPPGKGLEWMGR | 27 |
| | IRYNGDTSYNSALKSRLTISRDTSKNQVSLKLSSVTAADTAVYYCTRGPY | |
| | GGSREPLFDYWGQGTMVTVSS | |
| 153A6B1 VH.V5 | EVQLQESGPGLVKPSETLSLTCTVSGFSLTSYNIHWVRQPPGKGLEWMGR | 28 |
| | IRYNGDTSYNSALKSRLTISRDTSKNQVSLKLSSVTAADTAVYYCTRGPY | |
| | GGSREPLFDYWGQGTMVTVSS | |
| VH4-59-JH3 | QVQLQESGPGLVKPSETLSLTCTVSGGSISSYYWSWIRQPPGKGLEWIGY | 29 |
| (human | IYYSGSTNYNPSLKSRVTISVDTSKNQFSLKLSSVTAADTAVYYCAR--- | |
| germline) | ----------WGQGTMVTVSS | |
| 153A6B1 VK | DIKMTQSPSFLSAAVGDRVTINCKTSQNINKYLNWFQQKLGAAPKLLIYT | 2 |
| | TNNLQTGIPSRFSGSGSGTDFTLTISSLQPEDVATYFCLQHGSGYTFGAG | |
| | TKLELK | |
| 153A6B1 VK.V0 | DIQMTQSPSSLSASVGDRVTITCKTSQNINKYLNWYQQKPGKAPKLLIYT | 30 |
| (grafted) | TNNLQTGVPSRFSGSGSGTDFTLTISSLQPEDFATYYCLQHGSGYTFGQG | |
| | TKLEIK | |
| 153A6B1 VK.V1 | DIQMTQSPSSLSASVGDRVTITCKTSQNINKYLNWFQQKPGKAPKLLIYT | 31 |
| | TNNLQTGIPSRFSGSGSGTDFTLTISSLQPEDFATYYCLQHGSGYTFGQG | |
| | TKLEIK | |
| 153A6B1 VK.V2 | DIQMTQSPSSLSASVGDRVTITCKTSQNINKYLNWFQQKPGKAPKLLIYT | 32 |
| | TNNLQTGIPSRFSGSGSGTDFTLTISSLQPEDFATYFCLQHGSGYTFGQG | |
| | TKLEIK | |
| VK1-39(O12)-JK2 | DIQMTQSPSSLSASVGDRVTITCRASQSISSYLNWYQQKPGKAPKLLIYA | 33 |
| (human | ASSLQSGVPSRFSGSGSGTDFTLTISSLQPEDFATYYC--------FGQG | |
| germline) | TKLEIK | |

Table 4A. Humanized antibodies from 153A6B1

| | VK | | |
|---|---|---|---|
| VH | 153A6B1 VK.V0 | 153A6B1 VK.V1 | 153A6B1 VK.V2 |
| 153A6B1 VH.V0 | Hu153A6B1-1 | Hu153A6B1-7 | Hu153A6B1-13 |
| 153A6B1 VH.V1 | Hu153A6B1-2 | Hu153A6B1-8 | Hu153A6B1-14 |
| 153A6B1 VH.V2 | Hu153A6B1-3 | Hu153A6B1-9 | Hu153A6B1-15 |
| 153A6B1 VH.V3 | Hu153A6B1-4 | Hu153A6B1-10 | Hu153A6B1-16 |
| 153A6B1 VH.V4 | Hu153A6B1-5 | Hu153A6B1-11 | Hu153A6B1-17 |
| 153A6B1 VH.V5 | Hu153A6B1-6 | Hu153A6B1-12 | Hu153A6B1-18 |

TABLE 5
Humanization of 155A9B1

| | | SEQ |
|---|---|---|
| | | ID |
| Name | Sequence | NO: |
| 155A9B1 VH | QVQLKESGPGLVQPSQTLSLTCTVSGFSLTSYNVHWVRQPPGKGLEWMGR | 3 |
| | VRYNGDTSYNSALKSRLSISRDTSKNQAFLKMNSLQIDDTGTYYCTRGPY | |
| | GGSSEPLFDYWGQGVMVTVSS | |
| 155A9B1 VH- | QVQLKESGPGLVQPSQTLSLTCTVSGFSLTSYNVHWVRQPPGKGLEWMGR | 34 |
| G55A (CDR2 | VRYN [symbol] DTSYNSALKSRLSISRDTSKNQAFLKMNSLQIDDTGTYYCTRGPY | |
| mutated) | GGSSEPLFDYWGQGVMVTVSS | |
| 155A9B1 VH- | QVQLKESGPGLVQPSQTLSLTCTVSGFSLTSYNVHWVRQPPGKGLEWMGR | 35 |
| S61A (CDR2 | VRYNGDTSYN [symbol] ALKSRLSISRDTSKNQAFLKMNSLQIDDTGTYYCTRGPY | |
| mutated) | GGSSEPLFDYWGQGVMVTVSS | |
| 155A9B1 VH.V0 | EVQLQESGPGLVKPSETLSLTCTVSGGSISSYNVHWIRQPPGKGLEWIGR | 36 |
| (grafted) | VRYNGDTSYNSALKSRVTISVDTSKNQFSLKLSSVTAADTAVYYCARGPY | |
| | GGSSEPLFDYWGQGTMVTVSS | |
| 155A9B1 VH.V1 | EVQLQESGPGLVKPSETLSLTCTVSGGSISSYNVHWVRQPPGKGLEWIGR | 37 |
| | VRYNGDTSYNSALKSRVTISRDTSKNQFSLKLSSVTAADTAVYYCARGPY | |
| | GGSSEPLFDYWGQGTMVTVSS | |
| 155A9B1 VH.V2 | EVQLQESGPGLVKPSETLSLTCTVSGGSISSYNVHWVRQPPGKGLEWMGR | 38 |
| | VRYNGDTSYNSALKSRLTISRDTSKNQFSLKLSSVTAADTAVYYCARGPY | |
| | GGSSEPLFDYWGQGTMVTVSS | |
| 155A9B1 VH.V3 | EVQLQESGPGLVKPSETLSLTCTVSGGSISSYNVHWVRQPPGKGLEWMGR | 39 |
| | VRYNGDTSYNSALKSRLTISRDTSKNQFSLKLSSVTAADTAVYYCTRGPY | |
| | GGSSEPLFDYWGQGTMVTVSS | |
| 155A9B1 VH.V4 | EVQLQESGPGLVKPSETLSLTCTVSGFSISSYNVHWVRQPPGKGLEWMGR | 40 |
| | VRYNGDTSYNSALKSRLTISRDTSKNQASLKLSSVTAADTAVYYCTRGPY | |
| | GGSSEPLFDYWGQGTMVTVSS | |
| 155A9B1 VH.V5 | EVQLQESGPGLVKPSETLSLTCTVSGFSLTSYNVHWVRQPPGKGLEWMGR | 41 |
| | VRYNGDTSYNSALKSRLTISRDTSKNQASLKLSSVTAADTAVYYCTRGPY | |
| | GGSSEPLFDYWGQGTMVTVSS | |
| VH4-59-JH3 | QVQLQESGPGLVKPSETLSLTCTVSGGSISSYYWSWIRQPPGKGLEWIGY | 42 |
| (human | IYYSGSTNYNPSLKSRVTISVDTSKNQFSLKLSSVTAADTAVYYCAR--- | |
| germline) | ----------WGQGTMVTVSS | |
| 155A9B1 VK | DIKMTQSPSFLSASVGDRVTINCKASQNINKYLNWFQQKLGEAPRLLIYT | 4 |
| | TNNLQTGIPSRFSGSGSGTDFTLTISSLQPEDVATYFCLQHNSGYTFGAG | |
| | TKLELK | |
| 155A9B1 VK- | DIKMTQSPSFLSASVGDRVTINCKASQNINKYLNWFQQKLGEAPRLLIYT | 43 |
| S93A (CDR3 | TNNLQTGIPSRFSGSGSGTDFTLTISSLQPEDVATYFCLQHN [symbol] GYTFGAG | |
| mutated) | TKLELK | |
| 155A9B1 VK.V0 | DIQMTQSPSSLSASVGDRVTITCKASQNINKYLNWYQQKPGKAPRLLIYT | 44 |
| (grafted) | TNNLQTGVPSRFSGSGSGTDFTLTISSLQPEDFATYYCLQHNSGYTFGQG | |
| | TKLEIK | |
| 155A9B1 VK.V1 | DIQMTQSPSSLSASVGDRVTITCKASQNINKYLNWFQQKPGKAPRLLIYT | 45 |
| | TNNLQTGIPSRFSGSGSGTDFTLTISSLQPEDFATYYCLQHNSGYTFGQG | |
| | TKLEIK | |
| 155A9B1 VK.V2 | DIQMTQSPSSLSASVGDRVTITCKASQNINKYLNWFQQKPGKAPRLLIYT | 46 |
| | TNNLQTGIPSRFSGSGSGTDFTLTISSLQPEDFATYFCLQHNSGYTFGQG | |
| | TKLEIK | |
| VK1-39(O12)-JK2 | DIQMTQSPSSLSASVGDRVTITCRASQSISSYLNWYQQKPGKAPKLLIYA | 47 |
| (human | ASSLQSGVPSRFSGSGSGTDFTLTISSLQPEDFATYYC--------FGQG | |
| germline) | TKLEIK | |

Table 5A. Humanized antibodies from 155A9B1

| | VK | | | | |
|---|---|---|---|---|---|
| | 155A9B1 | 155A9B1 | 155A9B1 | 155A9B1 | 155A9B1 |
| VH | VK.V0 | VK.V1 | VK.V2 | VK-s93a | VK |
| 155A9B1 VH.V0 | Hu155A9B1-1 | Hu155A9B1-7 | Hu155A9B1-13 | | |
| 155A9B1 VH.V1 | Hu155A9B1-2 | Hu155A9B1-8 | Hu155A9B1-14 | | |
| 155A9B1 VH.V2 | Hu155A9B1-3 | Hu155A9B1-9 | Hu155A9B1-15 | | |
| 155A9B1 VH.V3 | Hu155A9B1-4 | Hu155A9B1-10 | Hu155A9B1-16 | | |
| 155A9B1 VH.V4 | Hu155A9B1-5 | Hu155A9B1-11 | Hu155A9B1-17 | | |
| 155A9B1 VH.V5 | Hu155A9B1-6 | Hu155A9B1-12 | Hu155A9B1-18 | | |
| 155A9B1 VH-G55A | | | | | 155A9B1-C1 |
| 155A9B1 VH-S61A | | | | | 155A9B1-C2 |
| 155A9B1 VH | | | | 155A9B1-C3 | 155A9B1-C |

TABLE 6
Humanization of 192A7B9

| | | SEQ |
|---|---|---|
| | | ID |
| Name | Sequence | NO: |
| 192A7B9 VH | QVQLKESGPGLVQPSQTLSLNCTVSGFSLTSYNVHWVRQPPGKGLEWMGR | 5 |
| | MRYNGDTSYNSALKSRLSISRDTSKNQVFLKMNSLQTDDTGTYYCTRGPR | |
| | GGYYSALFDYWGQGVMVTVSS | |
| 192A7B9 VH- | QVQLKESGPGLVQPSQTLSLNCTVSGFSLTSYNVHWVRQPPGKGLEWMGR | 48 |
| G55A (CDR2 | MRYN [symbol] DTSYNSALKSRLSISRDTSKNQVFLKMNSLQTDDTGTYYCTRGPR | |
| mutated) | GGYYSALFDYWGQGVMVTVSS | |
| 192A7B9 VH- | QVQLKESGPGLVQPSQTLSLNCTVSGFSLTSYNVHWVRQPPGKGLEWMGR | 49 |
| S61A (CDR2 | MRYNGDTSYN [symbol] ALKSRLSISRDTSKNQVFLKMNSLQTDDTGTYYCTRGPR | |
| mutated) | GGYYSALFDYWGQGVMVTVSS | |
| 192A7B9 VH.V0 | EVQLQESGPGLVKPSETLSLTCTVSGGSISSYNVHWIRQPPGKGLEWIGR | 50 |
| (grafted) | MRYNGDTSYNSALKSRVTISVDTSKNQFSLKLSSVTAADTAVYYCARGPR | |
| | GGYYSALFDYWGQGTMVTVSS | |
| 192A7B9 VH.V1 | EVQLQESGPGLVKPSETLSLTCTVSGGSISSYNVHWVRQPPGKGLEWIGR | 51 |
| | MRYNGDTSYNSALKSRVTISRDTSKNQFSLKLSSVTAADTAVYYCARGPR | |
| | GGYYSALFDYWGQGTMVTVSS | |
| 192A7B9 VH.V2 | EVQLQESGPGLVKPSETLSLTCTVSGGSISSYNVHWVRQPPGKGLEWMGR | 52 |
| | MRYNGDTSYNSALKSRLTISRDTSKNQFSLKLSSVTAADTAVYYCARGPR | |
| | GGYYSALFDYWGQGTMVTVSS | |
| 192A7B9 VH.V3 | EVQLQESGPGLVKPSETLSLTCTVSGGSISSYNVHWVRQPPGKGLEWMGR | 53 |
| | MRYNGDTSYNSALKSRLTISRDTSKNQFSLKLSSVTAADTAVYYCTRGPR | |
| | GGYYSALFDYWGQGTMVTVSS | |
| 192A7B9 VH.V4 | EVQLQESGPGLVKPSETLSLTCTVSGFSISSYNVHWVRQPPGKGLEWMGR | 54 |
| | MRYNGDTSYNSALKSRLTISRDTSKNQVSLKLSSVTAADTAVYYCTRGPR | |
| | GGYYSALFDYWGQGTMVTVSS | |
| 192A7B9 VH.V5 | EVQLQESGPGLVKPSETLSLTCTVSGFSLTSYNVHWVRQPPGKGLEWMGR | 55 |
| | MRYNGDTSYNSALKSRLTISRDTSKNQVSLKLSSVTAADTAVYYCTRGPR | |
| | GGYYSALFDYWGQGTMVTVSS | |
| VH4-59-JH3 | QVQLQESGPGLVKPSETLSLTCTVSGGSISSYYWSWIRQPPGKGLEWIGY | 56 |
| (human | IYYSGSTNYNPSLKSRVTISVDTSKNQFSLKLSSVTAADTAVYYCAR--- | |
| germline) | ----------WGQGTMVTVSS | |
| 192A7B9 VK | DIQMTQSPSFLSASVGDRVTINCKASQNINRYLNWYQQKLGDAPKVLISI | 6 |
| | ANSLQTGIPSRFSGSGSGTDFTLTISSLQPEDVATYFCLQHNSWYTFGAG | |
| | TKLELK | |
| 192A7B9 VK- | DIQMTQSPSFLSASVGDRVTINCKASQNINRYLNWYQQKLGDAPKVLISI | 57 |
| S53A (CDR2 | AN [symbol] LQTGIPSRFSGSGSGTDFTLTISSLQPEDVATYFCLQHNSWYTFGAG | |
| mutated) | TKLELK | |
| 192A7B9 VK- | DIQMTQSPSFLSASVGDRVTINCKASQNINRYLNWYQQKLGDAPKVLISI | 58 |
| S93A (CDR3 | ANSLQTGIPSRFSGSGSGTDFTLTISSLQPEDVATYFCLQHN [symbol] WYTFGAG | |
| mutated) | TKLELK | |
| 192A7B9 VK.V0 | DIQMTQSPSSLSASVGDRVTITCKASQNINRYLNWYQQKPGKAPKLLIYI | 59 |
| (grafted) | ANSLQTGVPSRFSGSGSGTDFTLTISSLQPEDFATYYCLQHNSWYTFGQG | |
| | TKLEIK | |
| 192A7B9 VK.V1 | DIQMTQSPSSLSASVGDRVTITCKASQNINRYLNWYQQKPGKAPKLLISI | 60 |
| | ANSLQTGVPSRFSGSGSGTDFTLTISSLQPEDFATYYCLQHNSWYTFGQG | |
| | TKLEIK | |
| 192A7B9 VK.V2 | DIQMTQSPSSLSASVGDRVTITCKASQNINRYLNWYQQKPGKAPKLLISI | 61 |
| | ANSLQTGIPSRFSGSGSGTDFTLTISSLQPEDFATYYCLQHNSWYTFGQG | |
| | TKLEIK | |
| 192A7B9 VK.V3 | DIQMTQSPSSLSASVGDRVTITCKASQNINRYLNWYQQKPGKAPKVLISI | 62 |
| | ANSLQTGIPSRFSGSGSGTDFTLTISSLQPEDFATYFCLQHNSWYTFGQG | |
| | TKLEIK | |
| VK1-39(O12)- | DIQMTQSPSSLSASVGDRVTITCRASQSISSYLNWYQQKPGKAPKLLIYA | 63 |
| JK2 | ASSLQSGVPSRFSGSGSGTDFTLTISSLQPEDFATYYC--------FGQG | |
| | TKLEIK | |

Table 6A. Humanized antibodies from 192A7B9

| | | VK | | | | | |
|---|---|---|---|---|---|---|---|
| | 192A7B9 | 192A7B9 | 192A7B9 | 192A7B9 | 192A7B9 | 192A7B9 | 192A7B9 |
| VH | VK.V0 | VK.V1 | VK.V2 | VK.V3 | VK-s53a | VK-s93a | VK |
| 192A7B9 | Hu192A7B9- | Hu192A7B9- | Hu192A7B9- | Hu192A7B9- | | | |
| VH.V0 | 1 | 7 | 13 | 19 | | | |
| 192A7B9 | Hu192A7B9- | Hu192A7B9- | Hu192A7B9- | Hu192A7B9- | | | |
| VH.V1 | 2 | 8 | 14 | 20 | | | |
| 192A7B9 | Hu192A7B9- | Hu192A7B9- | Hu192A7B9- | Hu192A7B9- | | | |
| VH.V2 | 3 | 9 | 15 | 21 | | | |
| 192A7B9 | Hu192A7B9- | Hu192A7B9- | Hu192A7B9- | Hu192A7B9- | | | |
| VH.V3 | 4 | 10 | 16 | 22 | | | |
| 192A7B9 | Hu192A7B9- | Hu192A7B9- | Hu192A7B9- | Hu192A7B9- | | | |
| VH.V4 | 5 | 11 | 17 | 23 | | | |
| 192A7B9 | Hu192A7B9- | Hu192A7B9- | Hu192A7B9- | Hu192A7B9- | | | |
| VH.V5 | 6 | 12 | 18 | 24 | | | |
| 192A7B9 | | | | | | | 192A7B9- |
| VH-G55A | | | | | | | C1 |
| 192A7B9 | | | | | | | 192A7B9- |
| VH-S61A | | | | | | | C2 |
| 192A7B9 | | | | | 192A7B9- | 192A7B9- | 192A7B9- |
| VH | | | | | C3 | C4 | C |

To compare the physical and chemical properties of CD3 humanized antibody with benchmark SP34, scFv form of indicated monoclonal antibody were purified from transiently transfected supernatant of the HEK293 cells by Protein A affinity column. The purity was examined by SEC-HPLC. Table 6B shows that 155A9B1-8-scFv hFc mono-antibodies had greater purity and yield in comparison with SP34. No SP34-scFv hFc antibody could be expressed in the same experimental condition.

TABLE 6B
Compare the purity and yield of
scFv form of 155A9B1-8 with SP34.

| | Purity | Yield (mg/L) |
|---|---|---|
| 155A9B1-8-scFv hFc | 100% | 221 |
| SP34-scFv hFc | Not available | Not available |

### Example 7. The Binding Activity of the Humanized Antibodies to CD3 Antigen ELISA Testing

To evaluate the binding activity of the humanized antibodies, the humanized CD3 antibodies were subjected to ELISA test.

Briefly, microtiter plates were coated with human CD3 protein at 1 μg/ml in PBS, 100 μl/well at 4° C. overnight, then blocked with 150 μl/well of 1% BSA. Three-fold dilutions of the humanized antibodies starting from 15 μg/ml were added to each well and incubated for 1 hour at 37° C. The plates were washed with PBS/Tween and then incubated with anti-Human IgG (H&L) (GOAT) Antibody Peroxidase Conjugated for 30 mins at 37° C. After washing, the plates were developed with TMB substrate and analyzed by spectrophotometer at OD 450 nm. As shown in Table 7, the humanized CD3 antibodies bound to human CD3 with different binding potency.

TABLE 7
Binding activity of the humanized antibodies

| | |
|---|---|
| Abs | EC50 (ng/ml) |

Table 7A. Hu153A6B1-1~Hu153A6B1-5

| | |
|---|---|
| 153A6B1C8 | 3.418 |
| Hu153A6B1-1 | 16.320 |
| Hu153A6B1-2 | 3.393 |
| Hu153A6B1-3 | 3.871 |
| Hu153A6B1-4 | 3.719 |
| Hu153A6B1-5 | 3.233 |
| Human IgG1 | NA |

Table 7B. Hu153A6B1-6~Hu153A6B1-11

| | |
|---|---|
| 153A6B1C8 | 2.910 |
| Hu153A6B1-6 | 2.992 |
| Hu153A6B1-7 | 6.782 |
| Hu153A6B1-8 | 2.953 |
| Hu153A6B1-9 | 3.351 |
| Hu153A6B1-10 | 3.436 |
| Hu153A6B1-11 | 3.048 |
| Human IgG1 | NA |

Table 7C. Hu153A6B1-12~Hu153A6B1-18

| | |
|---|---|
| 153A6B1C8 | 2.737 |
| Hu153A6B1-12 | 2.692 |
| Hu153A6B1-13 | 6.382 |
| Hu153A6B1-14 | 3.056 |
| Hu153A6B1-15 | 3.034 |
| Hu153A6B1-16 | 3.438 |
| Hu153A6B1-17 | 2.893 |
| Hu153A6B1-18 | 2.816 |

Table 7D. Hu155A9B1-1~Hu155A9B1-5

| | |
|---|---|
| 155A9B1-C | 2.699 |
| Hu155A9B1-1 | 428.400 |
| Hu155A9B1-2 | 26.420 |
| Hu155A9B1-3 | 32.170 |
| Hu155A9B1-4 | 16.410 |
| Hu155A9B1-5 | 3.265 |

Table 7E. Hu155A9B1-6~Hu155A9B1-11

| | |
|---|---|
| 155A9B1-C | 2.544 |
| Hu155A9B1-6 | 4.159 |
| Hu155A9B1-7 | 136.200 |
| Hu155A9B1-8 | 5.837 |
| Hu155A9B1-9 | 6.283 |
| Hu155A9B1-10 | 6.035 |
| Hu155A9B1-11 | 3.197 |
| Human IgG1 | NA |

Table 7F. Hu155A9B1-12~Hu155A9B1-18

| | |
|---|---|
| 155A9B1-C | 2.344 |
| Hu155A9B1-12 | 3.353 |
| Hu155A9B1-13 | 123.700 |
| Hu155A9B1-14 | 5.655 |
| Hu155A9B1-15 | 6.505 |
| Hu155A9B1-16 | 7.283 |
| Hu155A9B1-17 | 3.072 |
| Hu155A9B1-18 | 1.950 |

Table 7G. Hu192A7B9-1~Hu192A7B9-5

| | |
|---|---|
| 192A7B9-C | 2.853 |
| Hu192A7B9-1 | 148.400 |
| Hu192A7B9-2 | 194.000 |
| Hu192A7B9-3 | 86.200 |
| Hu192A7B9-4 | 22.820 |
| Hu192A7B9-5 | 5.517 |
| Human IgG1 | NA |

Table 7H. Hu192A7B9-6~Hu192A7B9-11

| | |
|---|---|
| 192A7B9-C | 2.161 |
| Hu192A7B9-6 | 8.585 |
| Hu192A7B9-7 | 22.560 |
| Hu192A7B9-8 | 6.425 |
| Hu192A7B9-9 | 5.584 |
| Hu192A7B9-10 | 5.077 |
| Hu192A7B9-11 | 7.945 |
| Human IgG1 | NA |

Table 71. Hu192A7B9-12~Hu192A7B9-17

| | |
|---|---|
| 192A7B9-C | 2.301 |
| Hu192A7B9-12 | 9.739 |
| Hu192A7B9-13 | 14.500 |
| Hu192A7B9-14 | 5.017 |
| Hu192A7B9-15 | 28.750 |
| Hu192A7B9-16 | 7.461 |
| Hu192A7B9-17 | 4.456 |
| Human IgG1 | NA |

Table 7J. Hu192A7B9-18~Hu192A7B9-24

| | |
|---|---|
| 192A7B9-C | 2.534 |
| Hu192A7B9-18 | 8.831 |
| Hu192A7B9-19 | 4.981 |
| Hu192A7B9-20 | 5.153 |
| Hu192A7B9-21 | 5.461 |
| Hu192A7B9-22 | 7.116 |
| Hu192A7B9-23 | 15.77 |
| Hu192A7B9-24 | 20.15 |

### Cell-Based Binding

FACS was used to evaluate the binding activity of the humanized antibodies on human PBMC.

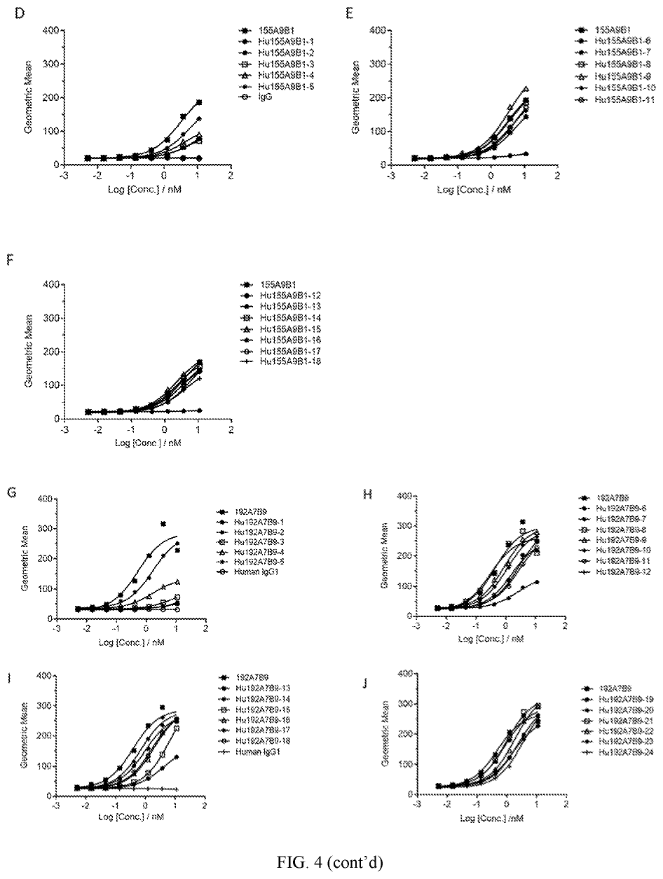

Briefly, huPBMC cells were firstly incubated with 3-fold serially diluted humanized CD3 antibodies at 4° C. for 30 mins. PE Goat anti-Human IgG Fc Secondary Antibody (eBioscience™, Invitrogen) was added to each well and incubated at 4° C. for 30 mins. Samples were washed twice with FACS buffer. The mean florescence intensity (MFI) of PE was evaluated by MACSQuant Analyzer 16. As shown in FIG. 4, the humanized CD3 antibodies bound to human PBMC with a variety of binding affinities.

### Example 8: Binding Affinity of the Humanized Antibodies

The binding of the humanized antibodies to recombinant CD3D&E protein (human CD3-his tag) was tested with Biacore using a capture method. The humanized antibodies were captured using Protein A chip. A serial dilution of human CD3-his tag protein was injected over captured antibody for 3 mins at a flow rate of 30 μl/min. The antigen was allowed to dissociate for 120-360 s. All the experiments were carried out on a Biacore T200. Data analysis was carried out using Biacore T200 evaluation software. The results are shown in Table 8.

TABLE 8
Affinity measured by Biacore

| | CD3d&ε-His | | |
|---|---|---|---|
| Abs | ka (1/Ms) | kd (1/s) | KD (M) |

Table 8A: 153A6B1 series

| | | | |
|---|---|---|---|
| 153A6B1 | 7.986E+4 | 6.939E−4 | 8.690E−9 |
| Hu153A6B1-1 | 4.442E+5 | 3.912E−3 | 8.808E−9 |
| Hu153A6B1-2 | 4.020E+5 | 2.011E−2 | 5.001E−8 |
| Hu153A6B1-3 | 4.060E+5 | 2.414E−2 | 5.945E−8 |
| Hu153A6B1-4 | 3.337E+5 | 1.953E−2 | 5.850E−8 |
| Hu153A6B1-5 | 9.132E+4 | 1.767E−3 | 1.935E−8 |
| Hu153A6B1-6 | 8.578E+4 | 1.810E−3 | 2.110E−8 |
| Hu153A6B1-7 | 3.137E+5 | 1.418E−2 | 4.519E−8 |
| Hu153A6B1-8 | 8.084E+5 | 3.957E−2 | 4.895E−8 |
| Hu153A6B1-9 | 5.576E+5 | 3.440E−2 | 6.169E−8 |
| Hu153A6B1-10 | 8.060E+5 | 5.678E−2 | 7.045E−8 |
| Hu153A6B1-11 | 9.303E+4 | 1.064E−3 | 1.143E−8 |
| Hu153A6B1-12 | 7.945E+4 | 1.072E−3 | 1.350E−8 |
| Hu153A6B1-13 | 3.488E+5 | 1.032E−2 | 2.959E−8 |
| Hu153A6B1-14 | 1.024E+6 | 5.300E−2 | 5.174E−8 |
| Hu153A6B1-15 | 1.978E+5 | 1.508E−2 | 7.621E−8 |
| Hu153A6B1-16 | 1.797E+6 | 1.113E−1 | 6.190E−8 |
| Hu153A6B1-17 | 9.545E+4 | 1.063E−3 | 1.114E−8 |
| Hu153A6B1-18 | 8.544E+4 | 1.069E−3 | 1.252E−8 |

Table 8B: 155A9B1 series

| | | | |
|---|---|---|---|
| 155A9B1 | 5.719E+4 | 2.068E−3 | 3.615E−8 |
| Hu155A9B1-1 | 1.093E+5 | 4.413E−3 | 4.038E−8 |
| Hu155A9B1-2 | 4.446E+5 | 7.037E−3 | 1.583E−8 |
| Hu155A9B1-3 | 3.700E+5 | 6.667E−3 | 1.802E−8 |
| Hu155A9B1-4 | 4.091E+5 | 6.477E−3 | 1.583E−8 |
| Hu155A9B1-5 | 1.036E+5 | 6.367E−3 | 6.144E−8 |
| Hu155A9B1-6 | 9.543E+4 | 5.926E−3 | 6.210E−8 |
| Hu155A9B1-7 | 4.065E+5 | 5.474E−3 | 1.347E−8 |
| Hu155A9B1-8 | 2.261E+5 | 1.588E−2 | 7.024E−8 |
| Hu155A9B1-9 | 2.822E+5 | 1.553E−2 | 5.505E−8 |
| Hu155A9B1-10 | 1.869E+5 | 1.505E−2 | 8.054E−8 |
| Hu155A9B1-11 | 6.929E+4 | 3.056E−3 | 4.410E−8 |
| Hu155A9B1-12 | 5.591E+4 | 2.863E−3 | 5.121E−8 |
| Hu155A9B1-13 | 2.804E+5 | 3.545E−3 | 1.264E−8 |
| Hu155A9B1-14 | 2.362E+5 | 1.872E−2 | 7.925E−8 |
| Hu155A9B1-15 | 2.851E+5 | 1.524E−2 | 5.345E−8 |
| Hu155A9B1-16 | 1.726E+5 | 1.650E−2 | 9.559E−8 |
| Hu155A9B1-17 | 7.040E+4 | 3.071E−3 | 4.363E−8 |
| Hu155A9B1-18 | 5.665E+4 | 2.910E−3 | 5.136E−8 |

Table 8C: 192A7B9 series

| | | | |
|---|---|---|---|
| 192A7B9 | 6.967E+4 | 4.416E−4 | 6.338E−9 |
| Hu192A7B9-1 | / | / | / |
| Hu192A7B9-2 | / | / | / |
| Hu192A7B9-3 | / | / | / |
| Hu192A7B9-4 | / | / | / |
| Hu192A7B9-5 | 1.159E+5 | 1.189E−2 | 1.026E−7 |
| Hu192A7B9-6 | 8.490E+4 | 2.400E−2 | 2.827E−7 |
| Hu192A7B9-7 | 1.897E+5 | 1.510E−2 | 7.963E−8 |
| Hu192A7B9-8 | 8.053E+5 | 4.747E−2 | 5.894E−8 |
| Hu192A7B9-9 | 2.199E+5 | 2.038E−2 | 9.265E−8 |
| Hu192A7B9-10 | 1.184E+5 | 1.445E−2 | 1.221E−7 |
| Hu192A7B9-11 | 5.784E+4 | 4.495E−3 | 7.772E−8 |
| Hu192A7B9-12 | 5.455E+4 | 3.171E−3 | 5.812E−8 |
| Hu192A7B9-13 | 2.864E+5 | 6.307E−3 | 2.202E−8 |
| Hu192A7B9-14 | 2.059E+5 | 1.786E−2 | 8.671E−8 |
| Hu192A7B9-15 | 1.812E+5 | 7.407E−3 | 4.089E−8 |
| Hu192A7B9-16 | 9.765E+4 | 1.429E−2 | 1.464E−7 |
| Hu192A7B9-17 | 8.221E+4 | 5.325E−3 | 6.478E−8 |
| Hu192A7B9-18 | 5.651E+4 | 3.168E−3 | 5.605E−8 |
| Hu192A7B9-19 | 1.573E+5 | 5.433E−3 | 3.453E−8 |
| Hu192A7B9-20 | 9.310E+4 | 5.610E−3 | 6.026E−8 |
| Hu192A7B9-21 | 9.060E+4 | 4.990E−3 | 5.507E−8 |
| Hu192A7B9-22 | 8.388E+4 | 4.617E−3 | 5.504E−8 |
| Hu192A7B9-23 | 7.025E+4 | 8.620E−4 | 1.227E−8 |
| Hu192A7B9-24 | 6.447E+4 | 5.940E−4 | 9.214E−9 |

### Example 9: CD3 NFAT Activity Induced by Humanized Antibodies

This example tested the functional activities of the humanized CD3 antibodies.

### Cell-Line Based Functional Characterization of CD3 Humanized Antibodies

To assess the ability of the CD3 humanized antibodies to activate the CD3 signaling pathway, a commercial CD3 NFAT luciferase reporter system was used. In this assay, Jurkat-CD3-NFAT was used as the reporter cell line. The Jurkat-CD3-NFAT cell line is genetically modified with CD3 downstream NFAT response element. Luciferase expression is induced upon antibody binding to the CD3 receptor. Briefly, reporter cells at a density of 2.5×10⁴ cells per well were cultured in a white 96-well plate. The test antibodies were 5-fold serially diluted added to a white 96-well assay plate, at a final concentration starting from 3 μg/ml. Following a 6 hours incubation at 37° C., luminescence was obtained by adding the substrate of luciferase and measured by a microplate reader. Four-parameter logistic curve analysis was performed with GraphPad software.

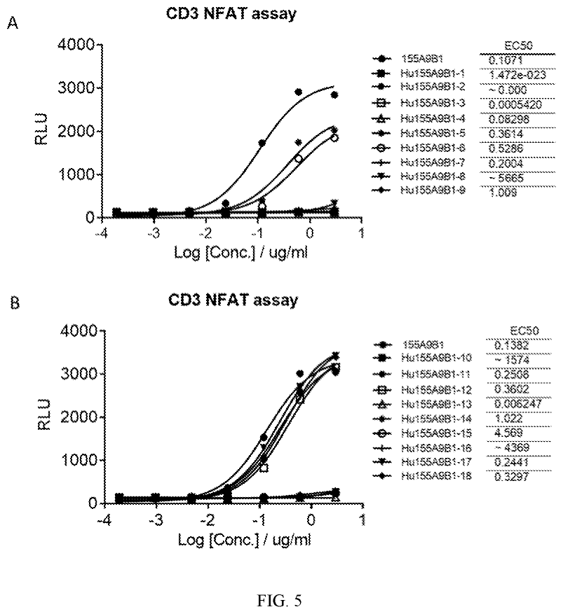

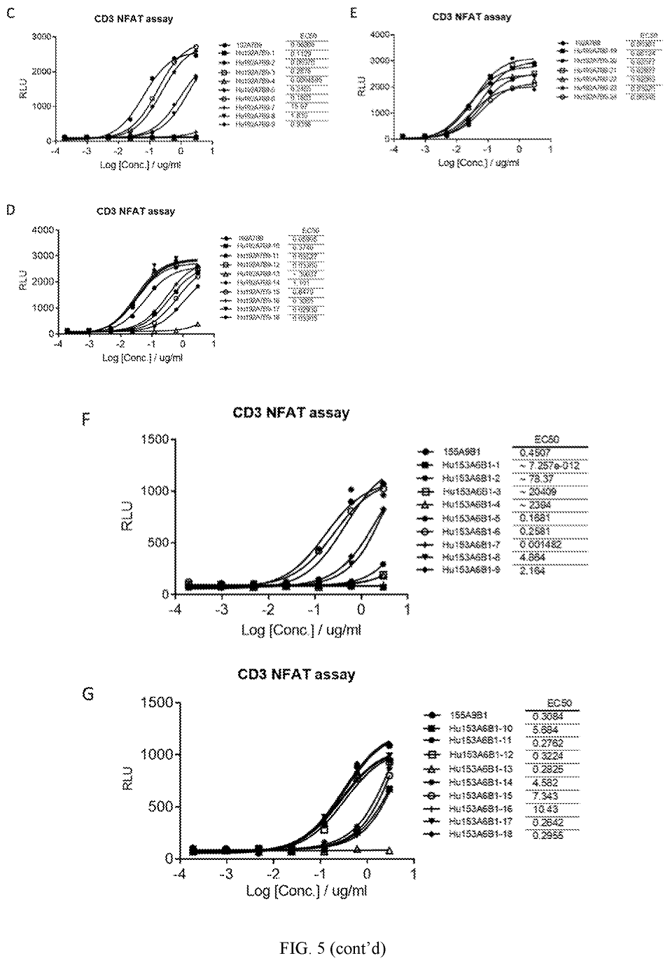

As shown in FIG. 5, the humanized CD3 antibodies showed a variety of different CD3 activities.

### Example 10: 4-1BB Expression Induced by Humanized Antibodies

This example assessed the ability of the CD3 humanized antibodies to activate 4-1BB expression.

Briefly, human PBMCs at a density of 1×10⁵ cells per well were cultured in a 96-well plate. Tested antibodies were 10-fold serially diluted and added to a 96-well plate, at a final concentration ranging from 20 nM to 0.0002 nM. Following a 48 hours incubation at 37° C., the human PBMCs were collected for further analysis. After washing by PBS, samples were stained using standard procedures by incubation in the dark at room temperature for 30 minutes with the following antibodies: anti-human CD4-APC (Ebiosciene, 17-0048-42), anti-human CD8-BV510 (BD bioscience, 563919), anti-human CD137(4-1BB)-PE (BD Pharmingen, 555956). Samples were washed twice with FACS buffer. After centrifugation, the supernatant was discarded, and the human PBMCs were resuspended in 0.2 mL FACS buffer. The CD137⁺ (4-1BB⁺) CD8⁺ T cell subsets amongst CD8⁺ T cells were evaluated by MACSQuant Analyzer 16. The percentage of CD137⁺ (4-1BB⁺) CD8⁺ T cells over total CD8⁺ T cells (%) was used to indicate the 4-1BB induction rate:

$$4 - 1 BB Induction Rate ( \% ) = \frac{\# of 4 - 1 {BB}^{+} CD {8}^{+} cells}{\# of total CD {8}^{+}} \% .$$

Interestingly, those humanized antibodies tested to have marginal or no response in NFAT tests (Grades 1-4) could be more clearly classified based on 4-1BB induction activities, and the 4-1BB induction rate is well correlated with CD3 NFAT activities (FIG. 6).

### Example 11: CD3 NFAT Activity Induced by CD3 Monoclonal Antibodies and Multispecific Antibodies

To further define CD3 potency, CD3 mediated NFAT activities were measured by using SP34 as the reference in TCR/CD3 Effector cells. The TCR/CD3 Effector cells (NFAT, Promega Cat #J1601) was used. When engaged with an anti-TCR/CD3 stimulus, receptor-mediated signaling induces luminescence (via activation of the NFAT) that can be detected by adding Bio-Glo™ Reagent and quantitating with a luminometer.

### CD3 NFAT Activity Induced by CD3 Monoclonal Antibodies

Briefly, the TCR/CD3 effector cells at a density of 2.5×10⁴ cells per well were cultured in a white 96-well plate. The CD3 humanized antibodies were 4-fold serially diluted added to a white 96-well assay plate, at a final concentration starting from 100 nM. Following a 6 hours incubation at 37° C., luminescence was obtained by adding the substrate of luciferase and measured by a microplate reader. Four-parameter logistic curve analysis was performed with GraphPad software.

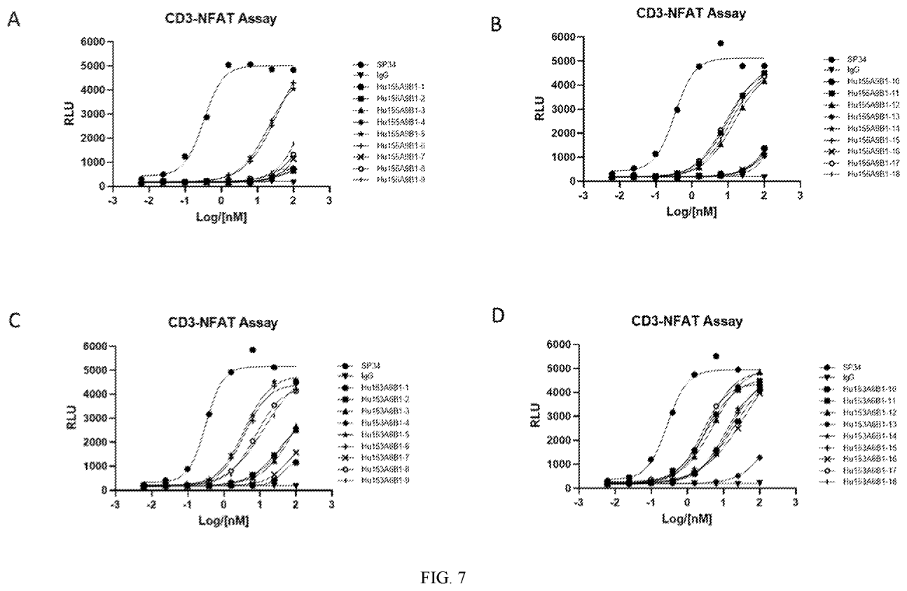

As shown in FIG. 7 and Table 10, the humanized CD3 antibodies showed a variety of different CD3 activities. Among them, the maximal effect (the highest luminescence readout, Emax) showed no more than 50% of SP34 in NFAT responses were defined as Grade 1-4. The maximal effect (the highest luminescence readout, Emax) showed more than 50% of SP34 in NFAT responses were defined as Grade 5-9. Table 9 showed the Top value (Emax) and relative percentage of that induced by SP34 in CD3-NFAT assays.

TABLE 9
The Emax and relative percentage of that induced by
SP34 of indicated antibodies in CD3-NFAT assays.

| | | Top value | % of |
|---|---|---|---|
| | Grade | (Emax) | SP34 |
| SP34 | — | 5856 | 100.00 |
| IgG | — | 208 | 3.55 |
| Hu153A6B1-1 | 1-4 | 1160 | 19.81 |
| Hu153A6B1-2 | 1- | 2496 | 42.62 |
| Hu153A6B1-3 | 1- | 2664 | 45.49 |
| Hu153A6B1-4 | 1- | 2584 | 44.13 |
| Hu153A6B1-5 | 5-9 | 4600 | 78.55 |
| Hu153A6B1-6 | 5-9 | 4208 | 71.86 |
| Hu153A6B1-7 | 1-4 | 1576 | 26.91 |
| Hu153A6B1-8 | 5-9 | 4128 | 70.49 |
| Hu153A6B1-9 | 5-9 | 4312 | 73.63 |
| SP34 | — | 5520 | 100.00 |
| IgG | — | 232 | 4.20 |
| Hu153A6B1-10 | 5-9 | 4080 | 73.91 |
| Hu153A6B1-11 | 5-9 | 4472 | 81.01 |
| Hu153A6B1-12 | 5-9 | 4832 | 87.54 |
| Hu153A6B1-13 | 1-4 | 1280 | 23.19 |
| Hu153A6B1-14 | 5-9 | 4136 | 74.93 |
| Hu153A6B1-15 | 5-9 | 4152 | 75.22 |
| Hu153A6B1-16 | 5-9 | 3968 | 71.88 |
| Hu153A6B1-17 | 5-9 | 4264 | 77.25 |
| Hu153A6B1-18 | 5-9 | 4904 | 88.84 |
| SP34 | — | 5064 | 100.00 |
| IgG | — | 216 | 4.27 |
| Hu155A9B1-1 | 1-4 | 720 | 14.22 |
| Hu155A9B1-2 | 1-4 | 656 | 12.95 |
| Hu155A9B1-3 | 1-4 | 648 | 12.80 |
| Hu155A9B1-4 | 1-4 | 752 | 14.85 |
| Hu155A9B1-5 | 5-9 | 4048 | 79.94 |
| Hu155A9B1-6 | 5-9 | 4296 | 84.83 |
| Hu155A9B1-7 | 1-4 | 1144 | 22.59 |
| Hu155A9B1-8 | 1-4 | 1328 | 26.22 |
| Hu155A9B1-9 | 1-4 | 1768 | 34.91 |
| SP34 | — | 5744 | 100.00 |
| IgG | — | 240 | 4.18 |
| Hu155A9B1-10 | 1-4 | 1376 | 23.96 |
| Hu155A9B1-11 | 5-9 | 4504 | 78.41 |
| Hu155A9B1-12 | 5-9 | 4160 | 72.42 |
| Hu155A9B1-13 | 1-4 | 1056 | 18.38 |
| Hu155A9B1-14 | 1-4 | 1096 | 19.08 |
| Hu155A9B1-15 | 1-4 | 1152 | 20.06 |
| Hu155A9B1-16 | 1-4 | 1240 | 21.59 |
| Hu155A9B1-17 | 5-9 | 4368 | 76.04 |
| Hu155A9B1-18 | 5-9 | 4248 | 73.96 |

The top value of the SP34 Group was set as 100% for each assay. The percentage of top value of indicated antibodies over top value of SP34 was used to indicate CD3 T cell activation activity:

$$Percentage ( \% ) = \frac{Top value of Indicated Antibodies}{Top value of SP 34} \% .$$

### CD3 NFAT Activity Induced by CD3 Multispecific Antibodies

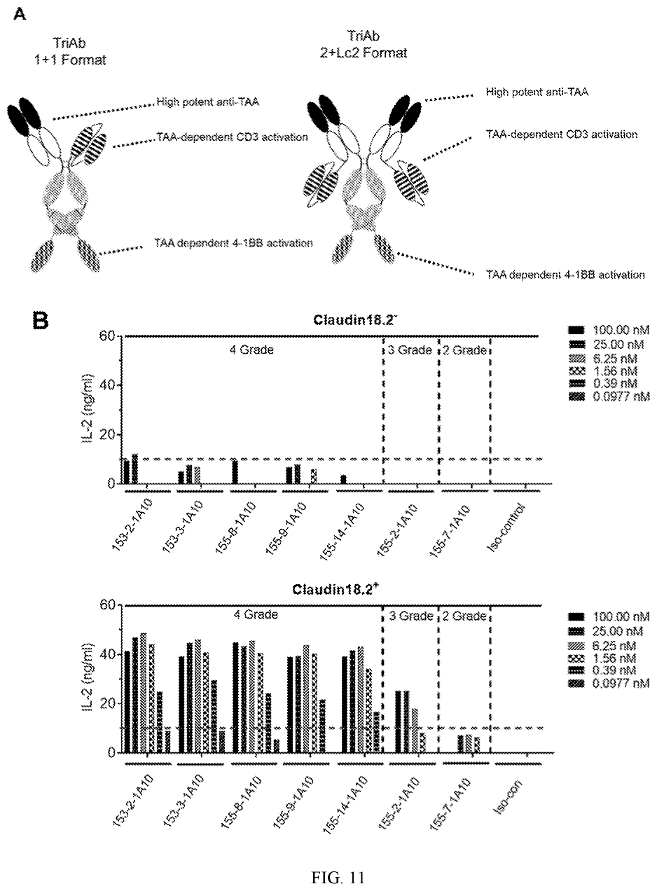

To further define the CD3 activities, CD3 multispecific antibodies were generated and the CD3 activity was further determined in the context of multispecific format in the presence of TAA positive cells. For bispecific antibodies, an anti-CD3 in scFv form fused to the N-terminal of Fc and anti-Claudin 18.2 or GPC3 in Fab form fused to another N-terminal of Fc were constructed into bispecific antibody in a 1+1 format (FIG. 9A). For trispecific antibodies, an anti-CD3 in scFv form fused to the N-terminal of heavy chain and 5T4 in Fab form fused to another N-terminal of Fc and an anti-4-1BB fused to the C-terminal end of each heavy chain were constructed into trispecific antibody 1+1 format (FIG. 11A). SP34 served as the reference control.

Briefly, the TCR/CD3 effector cells at a density of 2.5×10⁴ cells per well were cultured in a white 96-well plate. Target cell line that expressed Claudin 18.2 (CHO-K1-hCLDN18.2), 5T4 (CHO-K1-h5T4 and MCF7) or GPC3 (HepG2) were seeded at a density of 2.5×10⁴ (hence an E:T ratio of 1:1). The test CD3-bispecific or trispecific antibodies were serially diluted added to a white 96-well assay plate, at a final concentration starting from 100 nM. SP34 (Biointron, B6762) served as the benchmark controls. Following a 6-hour incubation at 37° C., luminescence was obtained by adding the substrate of luciferase and measured by a microplate reader. Four-parameter logistic curve analysis was performed with GraphPad software.

Surprisingly, all CD3 antibodies belongs to Grade 1-4 activity could be further defined and classified based on their TAA-dependent T cell activation activity that was measured in the assay mentioned above. Based on their TAA-dependent activity in comparison with the benchmark SP34 monoclonal antibody, anti-CD3 antibodies that having a stronger activity than SP34 monoclonal antibody were defined as Grade 3 and 4; while those anti-CD3 antibodies that having non-detectable activity were defined as Grade 1 and 2.

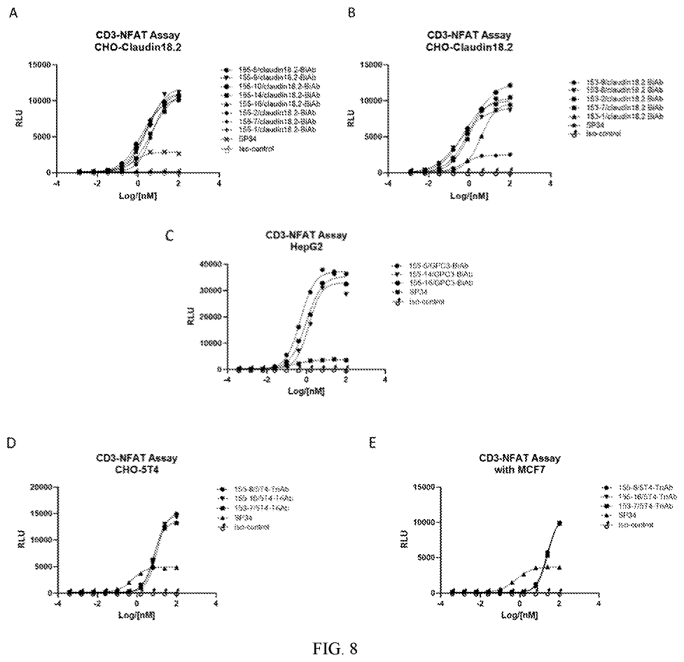

As shown in FIG. 8A-B, all CD3 antibodies that having Grade 1 or 2 activity, such as 155-7 and 155-1, had non-detectable CD3 activation in the presence of Claudin 18.2, while all other antibodies having an activity above Grade 2 level showed stronger CD3 activity, compared to SP34 monoclonal antibody. Similarly, as showed in FIG. 8C, all the tested CD3 antibodies having an activity above Grade 2 (for instance, 155-5 belongs to “Grade 5-9 activity”, 155-14 and 155-16 belong to “Grade 4 activity”) exhibited more stronger activity than that induced by SP34 monoclonal antibody, where these CD3 antibodies were constructed with GPC3-CD3 bispecific antibodies and in the presence of GPC3-expressing HepG2 cells. Again, consistently, all the tested CD3 antibodies having an activity above Grade 2 (for instance, 155-8 and 155-16 belong to “Grade 4 activity”, 153-7 in “Grade 3 activity”) also induced a more significant CD3 activation, compared to SP34 monoclonal antibody, in 5T4 high-expression cell lines, when these anti CD3 antibodies were constructed into 5T4×CD3 bispecific antibody (FIG. 8D-E).

Therefore, all antibodies above Grade 2 exhibited excellent TAA-dependent CD3 activities in the format of TAA-CD3 bispecific antibody. Grades 3 and 4 sequences stand out as having marginal or no CD3 agonism activity in terms of NFAT responses in the context of CD3 monoclonal antibody format and potent NFAT responses in the context of TAA-CD3 bispecific format when engaged with TAA-expressing cells. And meanwhile, the maximal effect (measured as the Emax) induced by Grade 3-4 sequences is at least more than that induced by SP34 antibody.

### Example 12 The Binding Activity of CD3/Claudin 18.2 Bispecific Antibodies to Human CD3 on Human PBMC

The following bispecific molecules were prepared in this example. An anti-CD3 antibody fragment from 155A9B1-8 in scFv form fused to the N-terminal and Claudin 18.2 in Fab form fused to another N-terminal of heavy chain were constructed into two different bispecific antibody formats A and B (FIG. 9A). Format A (also referred to as the “1+1 format”), represented by bispecific antibody 155-8A, includes an anti-CD3 scFv (on the right) in N-terminal and an anti-Claudin 18.2 Fab (on the left) in another terminal. Knob-in-hole substitutions are used in the Fc regions to reduce mispairing. Format B (also referred to as the “2+Lc2 format”), represented by bispecific antibody 155-8B, includes two anti-CD3 scFv fused to the C-terminus of each of the light chain variable regions of the anti-Claudin 18.2 Fab.

In addition to the two bispecific antibodies, 155-8A and 155-8B which include the scFv of humanized antibody 155-8, a third bispecific antibody was also prepared. This reference bispecific antibody, Xmab, adopted the 1+1 format and included a CD3 scFv from Plamotamab (Xencor).

FACS was used to evaluate the binding activity of the different formats of Claudin18.2/CD3 bispecific antibodies, and reference monospecific antibodies SP34, and 155-8, on human PBMC.

Briefly, huPBMCs were first incubated with 4-fold serially diluted claudin 18.2/CD3 bispecific antibodies starting at 100 nM at 4° C. for 30 mins. PE Goat anti-Human IgG Fc Secondary Antibody (eBioscience™, Invitrogen) was added to each well and incubated at 4° C. for 30 mins. Samples were washed twice with FACS buffer. The mean florescence intensity (MFI) of PE was evaluated by MACSQuant Analyzer 16.

The results are shown in FIG. 9B. The binding activity on huPBMC by bispecific antibodies 155-8A and 155-8B was weaker than the reference monospecific antibodies, as well as Xmab.

### Example 13: 4-1BB Expression Induced by CD3/Claudin 18.2 Bispecific Antibodies

This example measured the ability of the different formats of CD3/claudin 18.2 bispecific antibodies to activate 4-1BB expression,

Briefly, human PBMCs at a density of 1×10⁵ cells per well were cultured in a 96-well plate. Target cell line that expressed Claudin 18.2 (CHO-K1-hCLDN18.2) or control cells (CHO-K1) were seeded at a density of 2.5×10⁴ (hence an E:T ratio of 4:1). FACS analysis showed that the overexpression fold of Claudin18.2 in CHO-K1-hCLDN18.2 cells is about ninety compared to CHO-K1 cells. The FACS testing antibody is IMAB362 developed by Ganymed Pharmaceuticals AG. Then the tested bispecific antibodies were 4-fold serially diluted and added to a 96-well plate, at a final concentration ranging from 100 nM to 0.0004 nM. Following a 48 hours incubation at 37° C., human PBMCs were collected for further analysis. After washing by PBS, the samples were stained using standard procedures by incubation in the dark at room temperature for 30 minutes with the following antibodies: anti-human CD4-APC (Ebiosciene, 17-0048-42), anti-human CD8-BV510 (BD bioscience, 563919), anti-human CD137-PE (BD Pharmingen, 555956). Samples were washed twice with FACS buffer. After centrifugation, the supernatant was discarded, and the human PBMCs were resuspended in 0.2 mL FACS buffer. The CD137⁺ (4-1BB⁺)CD8⁺ T cell subsets amongst CD8⁺ T cells were evaluated by MACSQuant Analyzer 16.

The percentage of CD137⁺ (4-1BB⁺) CD8⁺ T cells over total CD8⁺ T cells (%) was used to indicate the 4-1BB induction rate. When tumor-associated antigen (Claudin 18.2)-expressing cells were used, a TAA-dependent 4-1BB induction rate was obtained; in the absence of these TAA-expressing cells (with CHO-K1 cells instead), a TAA-free 4-1BB induction rate was obtained.

FIG. 9C shows that 155-8A (1+1 format) induced more robust response than 155-8B (2+Lc2 Format). Also importantly, while SP34 and XmAb exhibited strong 4-1BB activation activities in both Claudin 18.2⁻ cells (CHO-K1) and Claudin 18.2⁺ cells, 155-8A and 155-8B's activities were significantly more pronounced in Claudin 18.2⁺ cells, indicating their dependency on claudin 18.2 expression.

### Example 14. IL-2 Secretion Induced by Claudin 18.2/4-1BB Bispecific Antibody is Dependent on CD3

This example examined whether the IL-2 secretion induced by Claudin 18.2/4-1BB bispecific antibody is dependent on CD3.

Briefly, the experiments were divided into two groups. In the first group, the plates were pre-coated with CD3 antibody (Clone HIT3a); in the second group, no CD3 antibody was used. Then human PBMCs at a density of 1×10⁵ cells per well were cultured in a 96-well plate. Target Cell line that expressed Claudin 18.2 (CHO-K1-hCLDN18.2) or control cells (CHO-K1) were seeded at a density of 2.5×10⁴ (hence an E:T ratio of 4:1). Claudin 18.2/4-1BB bispecific antibodies were 4-fold serially diluted and added to a 96-well assay plate, at a final concentration ranging from 100 nM to 0.0061 nM. Following a 48 hours incubation at 37° C., the supernatants were collected for further analysis. IL-2 was measured by TR-FRET assay (Perkin Elmer), followed by manufacturer's protocol. Lance signal was detected using Envision. Dual emission from 615 nM (channel 1) and 665 nM (channel 2) was acquired. Generated a standard curve by plotting the LANCE counts (Ch1/Ch2 ratio versus the concentration of standards). Data were analyzed using a nonlinear regression, 4-parameter logistic equation.

As shown in FIG. 10, 4-1BB-induced IL-2 secretion was indeed dependent on CD3 activation.

### Example 15. IL-2 Secretion Stimulated by Tri-Specific Antibodies

This example described a trispecific antibody that interacts with claudin 18.2, CD3 and 4-1BB to enhance both T cell activation and tumor targeting. This trispecific antibody binds to three targets: the protein claudin 18.2 on a tumor cell, and the proteins CD3 and 4-1BB on a T cell. The antibody's target-binding domains are illustrated in FIG. 11A. It includes a 1+1 format of anti-TAA/CD3 portion, with two anti-4-1BB nanobodies fused to the C-terminus of the Fc fragment.

To assess the ability of the CD3 trispecific antibodies to activate T cells, IL-2 secretion was examined by LANCE (Perkin Elmer). Briefly, human PBMC cells at a density of 1×10⁵ cells per well were cultured in a 96-well plate. Target Cell line that expressed Claudin 18.2 (CHO-K1-hCLDN18.2) or control cells (CHO-K1) were seeded at a density of 2.5×10⁴ (with an E:T ratio of 4:1). The test trispecific antibodies were 4-fold serially diluted and added to a 96-well plate, at a final concentration ranging from 100 nM to 0.098 nM. Following a 48 hours incubation at 37° C., the supernatants were collected for further analysis. IL2 was measured by a TR-FRET assay, following the manufacturer's protocol. Lance signal was detected using Envision. Dual emission from 615 nM (channel 1) and 665 nM (channel 2) was acquired. A standard curve was generated by plotting the LANCE counts (Ch1/Ch2 ratio versus the concentration of standards). The data were analyzed using a nonlinear regression, 4-parameter logistic equation.

In the presence of CHO-K1-hCLDN18.2 cells, IL-2 secretion (FIG. 11B) in human PBMC showed a positive correlation with CD3 NFAT activity. Grade 4B antibodies, including 155-8, 155-9, and 155-14 with marginal CD3 agonist activity, induced potent cytokine release without unspecific activation in CHO-K1 control cells. Grade 3 antibody (155-2) had non-detectable unspecific IL-2 activation in the absence of Claudin 18,2 even though the IL-2 activation in the presence of Claudin 18.2 was less pronounced. Therefore, both Grade 4 and Grade 3 antibodies exhibited excellent safety margins.

### Example 16. 4-1BB Expression Induced by Tri-Specific Antibodies

This example assessed the ability of the trispecific antibodies to activate 4-1BB expression.

Briefly, human PBMCs at a density of 1×10⁵ cells per well were cultured in a 96-well plate. Target cell line that expressed Claudin 18.2 (CHO-K1-hCLDN18.2) or control cells (CHO-K1) were seeded at a density of 2.5×10⁴ (hence an E:T ratio of 4:1). Tested antibodies were 4-fold serially diluted and added to a 96-well plate, at a final concentration ranging from 100 nM to 0.098 nM. Following a 48 hours incubation at 37° C., human PBMCs were collected for further analysis. After washing by PBS, the samples were stained using standard procedures by incubation in the dark at room temperature for 30 minutes with the following antibodies: anti-human CD4-APC (Ebiosciene, 17-0048-42), anti-human CD8-BV510 (BD bioscience, 563919), anti-human CD137-PE (BD Pharmingen, 555956). Samples were washed twice with FACS buffer. After centrifugation, the supernatant was discarded, and the human PBMCs were resuspended in 0.2 mL FACS buffer. The CD137⁺ (4-1BB⁺)CD8⁺ T cell subsets amongst CD8⁺ T cells were evaluated by MACSQuant Analyzer 16.

The percentage of CD137⁺ (4-1BB⁺) CD8⁺ T cells over total CD8⁺ T cells (%) was used to indicate the 4-1BB induction rate. When tumor-associated antigen (Claudin 18.2)-expressing cells were used, a TAA-dependent 4-1BB induction rate was obtained; in the absence of these TAA-expressing cells (with CHO-K1 cells instead), a TAA-free 4-1BB induction rate was obtained.

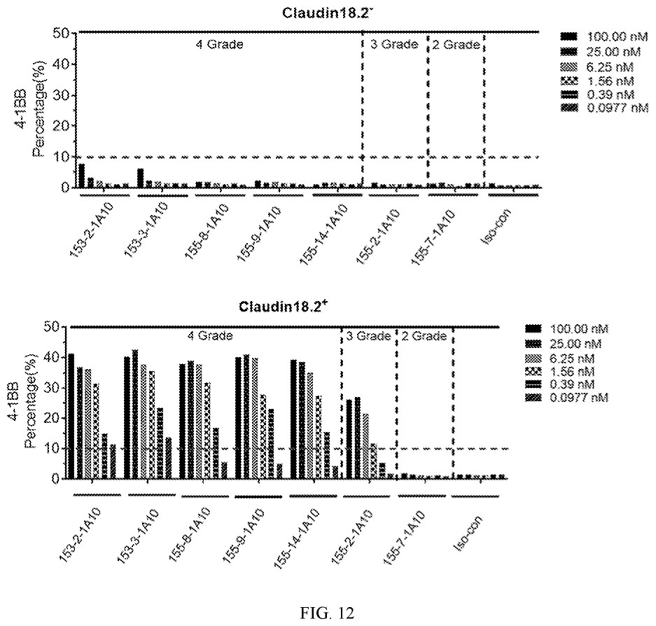

In the presence of CHO-K1-hCLDN18.2 cells, 4-1BB induction (FIG. 12) in human PBMC CD8⁺ cells showed a positive correlation with CD3 NFAT activity. Like in FIG. 10B, Grade 4B antibodies 155-8, 155-9, and 155-14 induced potent 4-1BB induction without unspecific activation in CHO-K1 control cells. Also, Grade 3 antibody (155-2) had non-detectable unspecific 4-1BB activation in the absence of Claudin 18,2 even though its activation in the presence of Claudin 18.2 was less pronounced. Again, both Grade 4 and Grade 3 antibodies exhibited excellent safety margins.

Based on the data from CD3-NFAT activities induced by CD3 unit without TAA (FIG. 7A-D), TAA-dependent T cell activation by TAA-CD3 bispecific antibodies (FIG. 8A-E) and TAA-dependent 4-1BB induction by CD3-4-1BB-TAA trispecific antibodies (FIG. 12), the CD3 activity could be divided into nine categories as shown in Table 10.

TABLE 10
Potency Categories

| | Assay | | | | | |
|---|---|---|---|---|---|---|
| | | TAA- | | | | |
| | | dependent | 4-1BB | | | |
| | | CD3-NFAT | Induction | | | |
| | CD3-NFAT | Activity by | by TAA × | | | |
| | Activity by | TAA × CD3 | CD3 × | | | |
| Category | CD3 mAb | BsAb | 4-1BB TriAb | 153 series | 155 series | 192 series |
| Grade 5-9 | >50% SP34 | >SP34 | ++++ | 153-5, 6, 17, | 155-11, 17, | 192-22, 21, 17, |
| | | | | 11, 18, 12, | 18, 12, 5, 6 | 23, 18, 12, 11, |
| | | | | 9, 8, 14, 10, | | 20, 19, 24, 6, 5, |
| | | | | 15, 16 | | 16, 10, 15, 9, |
| | | | | | | 14, 8 |
| Grade 4 | <=50% SP34 | >SP34 | +++ | 153-2, 3, 4 | 155-8, 9, 10, | 192-7, 13 |
| | | | | | 14, 15, 16 | |
| Grade 3 | <=50% SP34 | >SP34 | ++ | 153-7, 13, 1 | 155-2, 3, 4 | 192-4 |
| Grade 2 | <=50% SP34 | No response | + | | 155-7, 13 | |
| Grade 1 | <=50% SP34 | No response | − | | 155-1 | 192-1, 2, 3 |

### Example 17. IL-2 Secretion and Cell Lysis Activity Induced by Benchmark Bispecific Antibodies

To compare the CD3 activity of the candidate antibodies with commercial benchmark CD3 antibodies, this example constructed our CD3 and commercial benchmark CD3 sequences into bispecific 1+1 format as shown in FIG. 9A. Xencor-BiAb stands for the CD3 sequence acquired from Plamotamab (Xencor); Roche-BiAb stands for the CD3 sequence acquired from Mosunetuzumab (Roche); Amt-BiAb stands for the CD3 sequence acquired from Tarlatamab (Amgen); Amb-BiAb stands for the CD3 sequence acquired from Blinatumomab (Amgen); SP34-BiAb stands for the CD3 sequence acquired from SP34.

This example used IL-2 secretion and cell lysis activity as the readout of CD3 activity, then evaluated the effects of the bispecific CD3 antibodies on IL-2 production and cell lysis activity of human PBMC co-cultured with CHO-K1-hCLDN18.2 cells.

### IL-2 Secretion Induced by CD3 Bispecific Antibodies

To assess the ability of the CD3 bispecific antibodies to activate T cells, IL-2 secretion was examined by LANCE (Perkin Elmer).

Briefly, human PBMCs at a density of 1×10⁵ cells per well were cultured in a 96-well plate. Target Cell line that expressed Claudin 18.2 (CHO-K1-hCLDN18.2) or control cells (CHO-K1) were seeded at a density of 2.5×10⁴. Thus, E:T ratio is 4:1. Antibodies were 5-fold serially diluted and added to a 96-well plate, at a final concentration ranging from 100 nM to 0.032 nM. Following a 48 hours incubation at 37° C., the supernatants were collected for further analysis. IL2 was measured by TR-FRET assay, following the manufacturer's protocol. Lance signal was detected using Envision. Dual emission from 615 nM (channel 1) and 665 nM (channel 2) was acquired. A standard curve was generated by plotting the LANCE counts (Ch1/Ch2 ratio versus the concentration of standards. Data were analyzed using a nonlinear regression, 4-parameter logistic equation.

### Cell Lysis Activity Induced by CD3 Bispecific Antibodies

To assess the ability of the CD3 bispecific antibodies to activate T cells, lactate dehydrogenase (LDH) activity released from the cytosol of damaged cells was examined by Cytotoxicity Detection Kit^{PLUS} (Roche).

Briefly, CD8⁺ cells were isolated from human PBMC by CD8 isolation kit (Miltenyi Biotec Inc.). CD8⁺ cells at a density of 2×10⁵ cells per well were cultured in a 96-well plate. Target Cell line that expressed Claudin 18.2 (CHO-K1-hCLDN18.2) or control cells (CHO-K1) were seeded at a density of 1×10⁴. Thus, E:T ratio is 20:1. Antibodies were 4-fold serially diluted and added to a 96-well assay plate, at a final concentration ranging from 10 nM to 3.81E-05 nM. Following a 24 hours incubation at 37° C., the supernatants were collected for further analysis. The LDH were examined following the manufacturer's protocol. An ELISA reader was used to measure the absorbance of the samples at 492 nm.

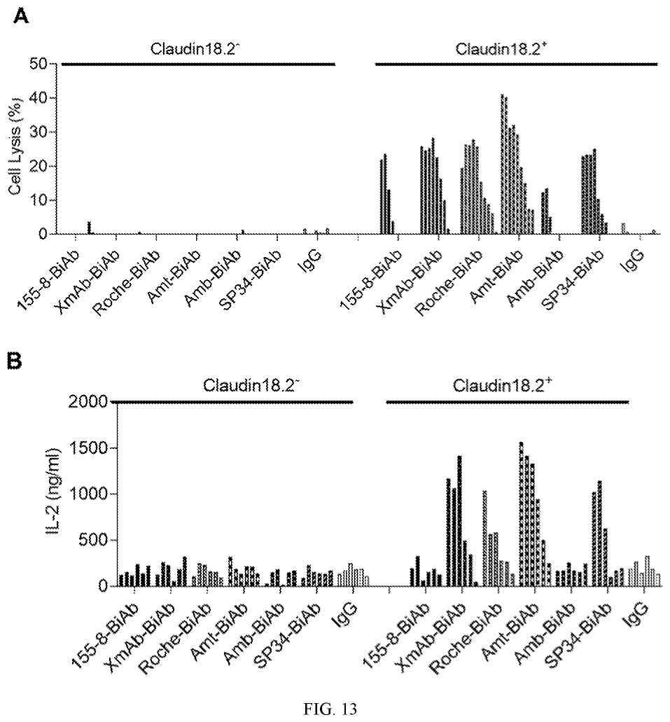

As shown in FIG. 13A, in the presence of CHO-K1-hCLDN18.2 cells, the test article 155-8 bispecific antibody showed less cell lysis activity than the benchmark bispecific antibodies, while in the presence of CHO-K1 cells, the 155-8 bispecific antibodies also triggered less unspecific cell lysis activity in contrast to benchmark bispecific antibodies.

While for IL-2 secretion (FIG. 13B), in the presence of CHO-K1-hCLDN18.2 cells, all the tested bispecific antibodies triggered comparable IL-2 secretion with isotype controls.

### Example 18. IL-2 Secretion and Cell Lysis Activity Induced by Benchmark Tri-Specific Antibodies

To compare the CD3 activity of our candidate with commercial benchmark CD3 antibodies, this example constructed our CD3 and commercial benchmark CD3 sequences into trispecific 1+1 format as in FIG. 11A. Xencor-TriAb stands for the CD3 sequence acquired from Plamotamab (Xencor); Roche-TriAb stands for the CD3 sequence acquired from Mosunetuzumab (Roche); Amt-TriAb stands for the CD3 sequence acquired from Tarlatamab (Amgen); Amb-TriAb stands for the CD3 sequence acquired from Blinatumomab (Amgen).We use IL-2 secretion and cell lysis activity as the readout of CD3 activity, then evaluated the effects of the trispecific CD3 antibodies on IL-2 production and cell lysis activity of human peripheral blood mononuclear cell (PBMC) co-cultured with CHO-K1-hCLDN18.2 cells.

### IL-2 Secretion Induced by CD3 Trispecific Antibodies

To assess the ability of the CD3 trispecific antibodies to activate T cells, IL-2 secretion was examined by LANCE (Perkin Elmer).

Briefly, human PBMCs at a density of 1×10⁵ cells per well were cultured in a 96-well plate. Target Cell line that expressed Claudin 18.2 (CHO-K1-hCLDN18.2) or control cells (CHO-K1) were seeded at a density of 2.5×10⁴. Thus, E:T ratio is 4:1. Antibodies were 5-fold serially diluted and added to a 96-well plate, at a final concentration ranging from 100 nM to 0.032 nM. Following a 48 hours incubation at 37° C., the supernatants were collected for further analysis. IL2 was measured by TR-FRET assay, followed by manufacturer's protocol. Lance signal was detected using Envision. Dual emission from 615 nM (channel 1) and 665 nM (channel 2) was acquired. A standard curve was generated by plotting the LANCE counts (Ch1/Ch2 ratio versus the concentration of standards). Data were analyzed using a nonlinear regression, 4-parameter logistic equation.

### Cell Lysis Activity Induced by CD3 Trispecific Antibodies

To assess the ability of the CD3 trispecific antibodies to activate T cells, lactate dehydrogenase (LDH) activity released from the cytosol of damaged cells was examined by Cytotoxicity Detection KitPLUS (Roche).

Briefly, human PBMCs at a density of 4×10⁵ cells per well were cultured in a 96-well plate. Target Cell line that expressed Claudin 18.2 (CHO-K1-hCLDN18.2) were seeded at a density of 2×10⁴. Thus, E:T ratio is 10:1. Antibodies were 5-fold serially diluted and added to a 96-well plate, at a final concentration ranging from 100 nM to 0.032 nM. Following a 48 hours incubation at 37° C., the supernatants were collected for further analysis. The LDH were examined followed by manufacturer's protocol. An ELISA reader was used to measure the absorbance of the samples at 492 nm.

As shown in FIG. 14A, in the presence of CHO-K1-hCLDN18.2 cells, the test article 155-8 trispecific antibody showed comparable cell lysis activity with trispecific antibodies by using other benchmark CD3 sequences, while in the presence of CHO-K1 cells, 155-8 trispecific antibody triggered marginal off-targeted cell lysis activity, which significantly less than other controls.

While for IL-2 secretion (FIG. 14B), in the presence of CHO-K1-hCLDN18.2 cells, the test article 155-8 Trispecific antibody induced robust IL-2 release from human PBMCs compared with benchmark trispecific antibodies. In the meantime, in the presence of CHO-K1 cells, 155-8 Trispecific antibody induced less unspecific IL-2 secretion in contrast to benchmark trispecific antibodies.

These results show that the CD3 activity of 155-8 was accurate enough to initiate TAA-dependent T cell activation in the 1+1 format of trispecific antibody.

### Example 19. In Vivo Tumor Treatment by Trispecific Antibodies

This example tested the efficacy of a trispecific antibody employing 155-8 and anti-5T4 (naptumomab, Active Biotech) and anti-4-1BB elements in treating B16F10-h5T4 tumors.

B16F10 is a mouse melanoma cell line derived from the pulmonary melanoma nodule. B16F10-h5T4 cells were injected into CD3/4-1BB humanized mice. Treatments started when the tumor size reached 100 mm³.

In addition to the 155-8 based trispecific antibody (CTM01-01), control treatments included two mutants of CTM01-01. One of the mutants, CTM01-01A included an inactivating mutation in the anti-4-1BB antibody, and the other, CTM01-01B, included an inactivating mutation in the anti-CD3 antibody.

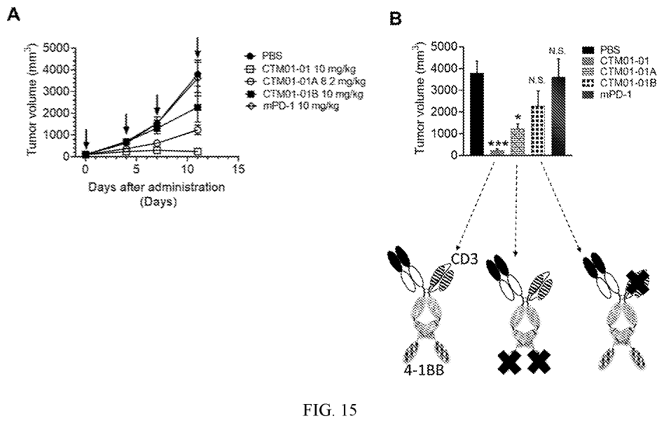

B16F10 tumor is a well-known PD-1 non-response and resistant model, widely considered one of the most difficult-to-treat tumors. The results are shown in FIG. 15. The mouse anti-PD-1 therapy had no efficacy in this model. The treatment with CTM01-01 (wild-type trispecific antibody targeting CD3, 4-1BB and human 5T4 as the TAA) resulted in dramatic tumor reduction. By contrast, the treatment with CTM01-01B, in which the anti-CD3 portion was inactivated, did not have a significant effect, and the treatment with CTM01-01A, in which the anti-4-1BB portion was inactivated, had only suboptimal efficacy. These results, therefore, demonstrate the exceptional in vivo efficacy of the anti-CD3/4-1BB/h5T4 trispecific antibodies.

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. An anti-CD3 antibody or antigen-binding fragment thereof having binding specificity to both human and cynomolgus CD3 proteins, wherein the antibody or antigen-binding fragment thereof comprises a heavy chain variable region (VH) comprising a VH CDR1, VH CDR2, and VH CDR3, and a light chain variable region (VL) comprising a VL CDR1, VL CDR2 and VL CDR3, wherein:
the VH CDR1 comprises the amino acid sequence of SEQ ID NO:7, the VH CDR2 comprises the amino acid sequence of SEQ ID NO:8, 64, 65, or 66, the VH CDR3 comprises the amino acid sequence of SEQ ID NO:9, the VL CDR1 comprises the amino acid sequence of SEQ ID NO:10, the VL CDR2 comprises the amino acid sequence of SEQ ID NO:11, and the VL CDR3 comprises the amino acid sequence of SEQ ID NO:12 or 67;
the VH CDR1 comprises the amino acid sequence of SEQ ID NO:13, the VH CDR2 comprises the amino acid sequence of SEQ ID NO:14, 68, 69 or 70, the VH CDR3 comprises the amino acid sequence of SEQ ID NO:15, the VL CDR1 comprises the amino acid sequence of SEQ ID NO:16, the VL CDR2 comprises the amino acid sequence of SEQ ID NO:11, and the VL CDR3 comprises the amino acid sequence of SEQ ID NO:17 or 71; or
the VH CDR1 comprises the amino acid sequence of SEQ ID NO:13, the VH CDR2 comprises the amino acid sequence of SEQ ID NO:18, 72, 73 or 74, the VH CDR3 comprises the amino acid sequence of SEQ ID NO:19, the VL CDR1 comprises the amino acid sequence of SEQ ID NO:20, the VL CDR2 comprises the amino acid sequence of SEQ ID NO:21 or 75, and the VL CDR3 comprises the amino acid sequence of SEQ ID NO:22 or 76.

2. The antibody or antigen-binding fragment thereof of claim 1, wherein:
the VH CDR1 comprises the amino acid sequence of SEQ ID NO:7, the VH CDR2 comprises the amino acid sequence of SEQ ID NO:8, the VH CDR3 comprises the amino acid sequence of SEQ ID NO:9, the VL CDR1 comprises the amino acid sequence of SEQ ID NO:10, the VL CDR2 comprises the amino acid sequence of SEQ ID NO:11, and the VL CDR3 comprises the amino acid sequence of SEQ ID NO:12;
the VH CDR1 comprises the amino acid sequence of SEQ ID NO:13, the VH CDR2 comprises the amino acid sequence of SEQ ID NO:14, the VH CDR3 comprises the amino acid sequence of SEQ ID NO:15, the VL CDR1 comprises the amino acid sequence of SEQ ID NO:16, the VL CDR2 comprises the amino acid sequence of SEQ ID NO:11, and the VL CDR3 comprises the amino acid sequence of SEQ ID NO:17; or
the VH CDR1 comprises the amino acid sequence of SEQ ID NO:13, the VH CDR2 comprises the amino acid sequence of SEQ ID NO:18, the VH CDR3 comprises the amino acid sequence of SEQ ID NO:19, the VL CDR1 comprises the amino acid sequence of SEQ ID NO:20, the VL CDR2 comprises the amino acid sequence of SEQ ID NO:21, and the VL CDR3 comprises the amino acid sequence of SEQ ID NO:22.

3. The antibody or antigen-binding fragment thereof of claim 1, wherein the VH CDR1 comprises the amino acid sequence of SEQ ID NO:7, the VH CDR2 comprises the amino acid sequence of SEQ ID NO:8, 64, 65, or 66, the VH CDR3 comprises the amino acid sequence of SEQ ID NO:9, the VL CDR1 comprises the amino acid sequence of SEQ ID NO:10, the VL CDR2 comprises the amino acid sequence of SEQ ID NO:11, and the VL CDR3 comprises the amino acid sequence of SEQ ID NO:12 or 67.

4. The antibody or antigen-binding fragment thereof of claim 3, wherein the VH comprises an amino acid sequence selected from the group consisting of SEQ ID NO:1 and 23-28, optionally with a G55A substitution, a S61A substitution, or the combination thereof, and the VL comprises an amino acid sequence selected from the group consisting of SEQ ID NO:2 and 30-32, optionally with a S93A substitution, all amino acid positions according to Kabat numbering.

5. The antibody or antigen-binding fragment thereof of claim 4, wherein:
the VH comprises the amino acid sequence of SEQ ID NO:24 and the VL comprises the amino acid sequence of SEQ ID NO:30;
the VH comprises the amino acid sequence of SEQ ID NO:25 and the VL comprises the amino acid sequence of SEQ ID NO:30; or
the VH comprises the amino acid sequence of SEQ ID NO:26 and the VL comprises the amino acid sequence of SEQ ID NO:30.

6. The antibody or antigen-binding fragment thereof of claim 4, wherein:
the VH comprises the amino acid sequence of SEQ ID NO:23 and the VL comprises the amino acid sequence of SEQ ID NO:31; or
the VH comprises the amino acid sequence of SEQ ID NO:23 and the VL comprises the amino acid sequence of SEQ ID NO:32.

7. The antibody or antigen-binding fragment thereof of claim 1, wherein the VH CDR1 comprises the amino acid sequence of SEQ ID NO:13, the VH CDR2 comprises the amino acid sequence of SEQ ID NO:14, 68, 69 or 70, the VH CDR3 comprises the amino acid sequence of SEQ ID NO:15, the VL CDR1 comprises the amino acid sequence of SEQ ID NO:16, the VL CDR2 comprises the amino acid sequence of SEQ ID NO:11, and the VL CDR3 comprises the amino acid sequence of SEQ ID NO:17 or 71.

8. The antibody or antigen-binding fragment thereof of claim 7, wherein the VH comprises an amino acid sequence selected from the group consisting of SEQ ID NO:3 and 36-41, optionally with a G55A substitution, a S61A substitution, or the combination thereof, and the VL comprises an amino acid sequence selected from the group consisting of SEQ ID NO:4 and 44-46, optionally with a S93A substitution, all amino acid positions according to Kabat numbering.

9. The antibody or antigen-binding fragment thereof of claim 8, wherein:
the VH comprises the amino acid sequence of SEQ ID NO:37 and the VL comprises the amino acid sequence of SEQ ID NO:45;
the VH comprises the amino acid sequence of SEQ ID NO:38 and the VL comprises the amino acid sequence of SEQ ID NO:45;
the VH comprises the amino acid sequence of SEQ ID NO:39 and the VL comprises the amino acid sequence of SEQ ID NO:45;
the VH comprises the amino acid sequence of SEQ ID NO:37 and the VL comprises the amino acid sequence of SEQ ID NO:46;
the VH comprises the amino acid sequence of SEQ ID NO:38 and the VL comprises the amino acid sequence of SEQ ID NO:46; or
the VH comprises the amino acid sequence of SEQ ID NO:39 and the VL comprises the amino acid sequence of SEQ ID NO:46.

10. The antibody or antigen-binding fragment thereof of claim 8, wherein the VH comprises the amino acid sequence of SEQ ID NO:37, optionally with a G55A substitution, a S61A substitution, or the combination thereof, and the VL comprises the amino acid sequence of SEQ ID NO:45, optionally with a S93A substitution, all amino acid positions according to Kabat numbering.

11. The antibody or antigen-binding fragment thereof of claim 8, wherein:
the VH comprises the amino acid sequence of SEQ ID NO:37 and the VL comprises the amino acid sequence of SEQ ID NO:44;
the VH comprises the amino acid sequence of SEQ ID NO:38 and the VL comprises the amino acid sequence of SEQ ID NO:44; or
the VH comprises the amino acid sequence of SEQ ID NO:39 and the VL comprises the amino acid sequence of SEQ ID NO:44.

12. The antibody or antigen-binding fragment thereof of claim 1, wherein the VH CDR1 comprises the amino acid sequence of SEQ ID NO:13, the VH CDR2 comprises the amino acid sequence of SEQ ID NO:18, 72, 73 or 74, the VH CDR3 comprises the amino acid sequence of SEQ ID NO:19, the VL CDR1 comprises the amino acid sequence of SEQ ID NO:20, the VL CDR2 comprises the amino acid sequence of SEQ ID NO:21 or 75, and the VL CDR3 comprises the amino acid sequence of SEQ ID NO:22 or 76.

13. The antibody or antigen-binding fragment thereof of claim 12, wherein the VH comprises an amino acid sequence selected from the group consisting of SEQ ID NO:5 and 50-55, optionally with a G55A substitution, a S61A substitution, or the combination thereof, and the VL comprises an amino acid sequence selected from the group consisting of SEQ ID NO:6 and 59-62, optionally with a S53A substitution, a S93A substitution, or the combination thereof, all amino acid positions according to Kabat numbering.

14. The antibody or antigen-binding fragment thereof of claim 12, wherein:
the VH comprises an amino acid sequence of SEQ ID NO:50 and the VL comprises an amino acid sequence of SEQ ID NO:60; or
the VH comprises an amino acid sequence of SEQ ID NO:50 and the VL comprises an amino acid sequence of SEQ ID NO:61.

15. The antibody or antigen-binding fragment thereof of claim 12, wherein the VH comprises an amino acid sequence of SEQ ID NO:53 and the VL comprises an amino acid sequence of SEQ ID NO:59.

16. (canceled)

17. A multispecific antibody comprising an anti-CD3 antigen-binding fragment of claim 1 and an anti-tumor associated antigen (TAA) unit.

18-19. (canceled)

20. The multispecific antibody of claim 17, wherein the TAA is selected from the group consisting of Claudin 18.2, GPC3, 5T4, EGFR, Her2, EpCAM, CD20, CD30, CD33, CD47, CD52, CD133, CD73, CEA, gpA33, Mucins, TAG-72, CIX, PSMA, folate-binding protein, GD2, GD3, GM2, VEGF, VEGFR, Integrin, αVβ3, α5β1, ERBB2, ERBB3, MET, IGF1R, EPHA3, TRAILR1, TRAILR2, RANKL, FAP and Tenascin.

21. One or more polynucleotide(s) encoding the antibody or fragment of claim 1.

22-23. (canceled)

24. A method for treating cancer, comprising administering a cancer patient an effective amount of the antibody or fragment of claim 1.

25. (canceled)

26. The method of claim 24, wherein the cancer is prostate cancer, pancreatic cancer, leukemia, breast cancer, colorectal cancer, gastric cancer, head and neck cancer, liver cancer, esophageal cancer, cervical cancer, thyroid cancer, lung cancer, bladder cancer, kidney cancer, uterus cancer, or melanoma.


SEQUENCE LISTING (79 sequences)

SEQ ID NO: 1 - protein, 121 aa, synthetic construct
QVQLKESGPGLVQPSQTLSLTCTVSGFSLTSYNIHWVRQPPGKGLEWMGRIRYNGDTSYNSALKSRLSISRDTSKNQVFLKMSSLQTDDTGTYYCTRGPYGGSREPLFDYWGQGVMVTVSS

SEQ ID NO: 2 - protein, 106 aa, synthetic construct
DIKMTQSPSFLSAAVGDRVTINCKTSQNINKYLNWFQQKLGAAPKLLIYTTNNLQTGIPSRFSGSGSGTDFTLTISSLQPEDVATYFCLQHGSGYTFGAGTKLELK

SEQ ID NO: 3 - protein, 121 aa, synthetic construct
QVQLKESGPGLVQPSQTLSLTCTVSGFSLTSYNVHWVRQPPGKGLEWMGRVRYNGDTSYNSALKSRLSISRDTSKNQAFLKMNSLQIDDTGTYYCTRGPYGGSSEPLFDYWGQGVMVTVSS

SEQ ID NO: 4 - protein, 106 aa, synthetic construct
DIKMTQSPSFLSASVGDRVTINCKASQNINKYLNWFQQKLGEAPRLLIYTTNNLQTGIPSRFSGSGSGTDFTLTISSLQPEDVATYFCLQHNSGYTFGAGTKLELK

SEQ ID NO: 5 - protein, 121 aa, synthetic construct
QVQLKESGPGLVQPSQTLSLNCTVSGFSLTSYNVHWVRQPPGKGLEWMGRMRYNGDTSYNSALKSRLSISRDTSKNQVFLKMNSLQTDDTGTYYCTRGPRGGYYSALFDYWGQGVMVTVSS

SEQ ID NO: 6 - protein, 106 aa, synthetic construct
DIQMTQSPSFLSASVGDRVTINCKASQNINRYLNWYQQKLGDAPKVLISIANSLQTGIPSRFSGSGSGTDFTLTISSLQPEDVATYFCLQHNSWYTFGAGTKLELK

SEQ ID NO: 7 - protein, 5 aa, synthetic construct
SYNIH

SEQ ID NO: 8 - protein, 16 aa, synthetic construct
RIRYNGDTSYNSALKS

SEQ ID NO: 9 - protein, 13 aa, synthetic construct
GPYGGSREPLFDY

SEQ ID NO: 10 - protein, 11 aa, synthetic construct
KTSQNINKYLN

SEQ ID NO: 11 - protein, 7 aa, synthetic construct
TTNNLQT

SEQ ID NO: 12 - protein, 8 aa, synthetic construct
LQHGSGYT

SEQ ID NO: 13 - protein, 5 aa, synthetic construct
SYNVH

SEQ ID NO: 14 - protein, 16 aa, synthetic construct
RVRYNGDTSYNSALKS

SEQ ID NO: 15 - protein, 13 aa, synthetic construct
GPYGGSSEPLFDY

SEQ ID NO: 16 - protein, 11 aa, synthetic construct
KASQNINKYLN

SEQ ID NO: 17 - protein, 8 aa, synthetic construct
LQHNSGYT

SEQ ID NO: 18 - protein, 16 aa, synthetic construct
RMRYNGDTSYNSALKS

SEQ ID NO: 19 - protein, 13 aa, synthetic construct
GPRGGYYSALFDY

SEQ ID NO: 20 - protein, 11 aa, synthetic construct
KASQNINRYLN

SEQ ID NO: 21 - protein, 7 aa, synthetic construct
IANSLQT

SEQ ID NO: 22 - protein, 8 aa, synthetic construct
LQHNSWYT

SEQ ID NO: 23 - protein, 121 aa, synthetic construct
EVQLQESGPGLVKPSETLSLTCTVSGGSISSYNIHWIRQPPGKGLEWIGRIRYNGDTSYNSALKSRVTISVDTSKNQFSLKLSSVTAADTAVYYCARGPYGGSREPLFDYWGQGTMVTVSS

SEQ ID NO: 24 - protein, 121 aa, synthetic construct
EVQLQESGPGLVKPSETLSLTCTVSGGSISSYNIHWVRQPPGKGLEWIGRIRYNGDTSYNSALKSRVTISRDTSKNQFSLKLSSVTAADTAVYYCARGPYGGSREPLFDYWGQGTMVTVSS

SEQ ID NO: 25 - protein, 121 aa, synthetic construct
EVQLQESGPGLVKPSETLSLTCTVSGGSISSYNIHWVRQPPGKGLEWMGRIRYNGDTSYNSALKSRLTISRDTSKNQFSLKLSSVTAADTAVYYCARGPYGGSREPLFDYWGQGTMVTVSS

SEQ ID NO: 26 - protein, 121 aa, synthetic construct
EVQLQESGPGLVKPSETLSLTCTVSGGSISSYNIHWVRQPPGKGLEWMGRIRYNGDTSYNSALKSRLTISRDTSKNQFSLKLSSVTAADTAVYYCTRGPYGGSREPLFDYWGQGTMVTVSS

SEQ ID NO: 27 - protein, 121 aa, synthetic construct
EVQLQESGPGLVKPSETLSLTCTVSGFSISSYNIHWVRQPPGKGLEWMGRIRYNGDTSYNSALKSRLTISRDTSKNQVSLKLSSVTAADTAVYYCTRGPYGGSREPLFDYWGQGTMVTVSS

SEQ ID NO: 28 - protein, 121 aa, synthetic construct
EVQLQESGPGLVKPSETLSLTCTVSGFSLTSYNIHWVRQPPGKGLEWMGRIRYNGDTSYNSALKSRLTISRDTSKNQVSLKLSSVTAADTAVYYCTRGPYGGSREPLFDYWGQGTMVTVSS

SEQ ID NO: 29 - protein, 108 aa, synthetic construct
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARWGQGTMVTVSS

SEQ ID NO: 30 - protein, 106 aa, synthetic construct
DIQMTQSPSSLSASVGDRVTITCKTSQNINKYLNWYQQKPGKAPKLLIYTTNNLQTGVPSRFSGSGSGTDFTLTISSLQPEDFATYYCLQHGSGYTFGQGTKLEIK

SEQ ID NO: 31 - protein, 106 aa, synthetic construct
DIQMTQSPSSLSASVGDRVTITCKTSQNINKYLNWFQQKPGKAPKLLIYTTNNLQTGIPSRFSGSGSGTDFTLTISSLQPEDFATYYCLQHGSGYTFGQGTKLEIK

SEQ ID NO: 32 - protein, 106 aa, synthetic construct
DIQMTQSPSSLSASVGDRVTITCKTSQNINKYLNWFQQKPGKAPKLLIYTTNNLQTGIPSRFSGSGSGTDFTLTISSLQPEDFATYFCLQHGSGYTFGQGTKLEIK

SEQ ID NO: 33 - protein, 98 aa, synthetic construct
DIQMTQSPSSLSASVGDRVTITCRASQSISSYLNWYQQKPGKAPKLLIYAASSLQSGVPSRFSGSGSGTDFTLTISSLQPEDFATYYCFGQGTKLEIK

SEQ ID NO: 34 - protein, 121 aa, synthetic construct
QVQLKESGPGLVQPSQTLSLTCTVSGFSLTSYNVHWVRQPPGKGLEWMGRVRYNADTSYNSALKSRLSISRDTSKNQAFLKMNSLQIDDTGTYYCTRGPYGGSSEPLFDYWGQGVMVTVSS

SEQ ID NO: 35 - protein, 121 aa, synthetic construct
QVQLKESGPGLVQPSQTLSLTCTVSGFSLTSYNVHWVRQPPGKGLEWMGRVRYNGDTSYNAALKSRLSISRDTSKNQAFLKMNSLQIDDTGTYYCTRGPYGGSSEPLFDYWGQGVMVTVSS

SEQ ID NO: 36 - protein, 121 aa, synthetic construct
EVQLQESGPGLVKPSETLSLTCTVSGGSISSYNVHWIRQPPGKGLEWIGRVRYNGDTSYNSALKSRVTISVDTSKNQFSLKLSSVTAADTAVYYCARGPYGGSSEPLFDYWGQGTMVTVSS

SEQ ID NO: 37 - protein, 121 aa, synthetic construct
EVQLQESGPGLVKPSETLSLTCTVSGGSISSYNVHWVRQPPGKGLEWIGRVRYNGDTSYNSALKSRVTISRDTSKNQFSLKLSSVTAADTAVYYCARGPYGGSSEPLFDYWGQGTMVTVSS

SEQ ID NO: 38 - protein, 121 aa, synthetic construct
EVQLQESGPGLVKPSETLSLTCTVSGGSISSYNVHWVRQPPGKGLEWMGRVRYNGDTSYNSALKSRLTISRDTSKNQFSLKLSSVTAADTAVYYCARGPYGGSSEPLFDYWGQGTMVTVSS

SEQ ID NO: 39 - protein, 121 aa, synthetic construct
EVQLQESGPGLVKPSETLSLTCTVSGGSISSYNVHWVRQPPGKGLEWMGRVRYNGDTSYNSALKSRLTISRDTSKNQFSLKLSSVTAADTAVYYCTRGPYGGSSEPLFDYWGQGTMVTVSS

SEQ ID NO: 40 - protein, 121 aa, synthetic construct
EVQLQESGPGLVKPSETLSLTCTVSGFSISSYNVHWVRQPPGKGLEWMGRVRYNGDTSYNSALKSRLTISRDTSKNQASLKLSSVTAADTAVYYCTRGPYGGSSEPLFDYWGQGTMVTVSS

SEQ ID NO: 41 - protein, 121 aa, synthetic construct
EVQLQESGPGLVKPSETLSLTCTVSGFSLTSYNVHWVRQPPGKGLEWMGRVRYNGDTSYNSALKSRLTISRDTSKNQASLKLSSVTAADTAVYYCTRGPYGGSSEPLFDYWGQGTMVTVSS

SEQ ID NO: 42 - protein, 108 aa, synthetic construct; identical to SEQ ID NO: 29

SEQ ID NO: 43 - protein, 106 aa, synthetic construct
DIKMTQSPSFLSASVGDRVTINCKASQNINKYLNWFQQKLGEAPRLLIYTTNNLQTGIPSRFSGSGSGTDFTLTISSLQPEDVATYFCLQHNAGYTFGAGTKLELK

SEQ ID NO: 44 - protein, 106 aa, synthetic construct
DIQMTQSPSSLSASVGDRVTITCKASQNINKYLNWYQQKPGKAPRLLIYTTNNLQTGVPSRFSGSGSGTDFTLTISSLQPEDFATYYCLQHNSGYTFGQGTKLEIK

SEQ ID NO: 45 - protein, 106 aa, synthetic construct
DIQMTQSPSSLSASVGDRVTITCKASQNINKYLNWFQQKPGKAPRLLIYTTNNLQTGIPSRFSGSGSGTDFTLTISSLQPEDFATYYCLQHNSGYTFGQGTKLEIK

SEQ ID NO: 46 - protein, 106 aa, synthetic construct
DIQMTQSPSSLSASVGDRVTITCKASQNINKYLNWFQQKPGKAPRLLIYTTNNLQTGIPSRFSGSGSGTDFTLTISSLQPEDFATYFCLQHNSGYTFGQGTKLEIK

SEQ ID NO: 47 - protein, 98 aa, synthetic construct; identical to SEQ ID NO: 33

SEQ ID NO: 48 - protein, 121 aa, synthetic construct
QVQLKESGPGLVQPSQTLSLNCTVSGFSLTSYNVHWVRQPPGKGLEWMGRMRYNADTSYNSALKSRLSISRDTSKNQVFLKMNSLQTDDTGTYYCTRGPRGGYYSALFDYWGQGVMVTVSS

SEQ ID NO: 49 - protein, 121 aa, synthetic construct
QVQLKESGPGLVQPSQTLSLNCTVSGFSLTSYNVHWVRQPPGKGLEWMGRMRYNGDTSYNAALKSRLSISRDTSKNQVFLKMNSLQTDDTGTYYCTRGPRGGYYSALFDYWGQGVMVTVSS

SEQ ID NO: 50 - protein, 121 aa, synthetic construct
EVQLQESGPGLVKPSETLSLTCTVSGGSISSYNVHWIRQPPGKGLEWIGRMRYNGDTSYNSALKSRVTISVDTSKNQFSLKLSSVTAADTAVYYCARGPRGGYYSALFDYWGQGTMVTVSS

SEQ ID NO: 51 - protein, 121 aa, synthetic construct
EVQLQESGPGLVKPSETLSLTCTVSGGSISSYNVHWVRQPPGKGLEWIGRMRYNGDTSYNSALKSRVTISRDTSKNQFSLKLSSVTAADTAVYYCARGPRGGYYSALFDYWGQGTMVTVSS

SEQ ID NO: 52 - protein, 121 aa, synthetic construct
EVQLQESGPGLVKPSETLSLTCTVSGGSISSYNVHWVRQPPGKGLEWMGRMRYNGDTSYNSALKSRLTISRDTSKNQFSLKLSSVTAADTAVYYCARGPRGGYYSALFDYWGQGTMVTVSS

SEQ ID NO: 53 - protein, 121 aa, synthetic construct
EVQLQESGPGLVKPSETLSLTCTVSGGSISSYNVHWVRQPPGKGLEWMGRMRYNGDTSYNSALKSRLTISRDTSKNQFSLKLSSVTAADTAVYYCTRGPRGGYYSALFDYWGQGTMVTVSS

SEQ ID NO: 54 - protein, 121 aa, synthetic construct
EVQLQESGPGLVKPSETLSLTCTVSGFSISSYNVHWVRQPPGKGLEWMGRMRYNGDTSYNSALKSRLTISRDTSKNQVSLKLSSVTAADTAVYYCTRGPRGGYYSALFDYWGQGTMVTVSS

SEQ ID NO: 55 - protein, 121 aa, synthetic construct
EVQLQESGPGLVKPSETLSLTCTVSGFSLTSYNVHWVRQPPGKGLEWMGRMRYNGDTSYNSALKSRLTISRDTSKNQVSLKLSSVTAADTAVYYCTRGPRGGYYSALFDYWGQGTMVTVSS

SEQ ID NO: 56 - protein, 108 aa, synthetic construct; identical to SEQ ID NO: 29

SEQ ID NO: 57 - protein, 106 aa, synthetic construct
DIQMTQSPSFLSASVGDRVTINCKASQNINRYLNWYQQKLGDAPKVLISIANALQTGIPSRFSGSGSGTDFTLTISSLQPEDVATYFCLQHNSWYTFGAGTKLELK

SEQ ID NO: 58 - protein, 106 aa, synthetic construct
DIQMTQSPSFLSASVGDRVTINCKASQNINRYLNWYQQKLGDAPKVLISIANSLQTGIPSRFSGSGSGTDFTLTISSLQPEDVATYFCLQHNAWYTFGAGTKLELK

SEQ ID NO: 59 - protein, 106 aa, synthetic construct
DIQMTQSPSSLSASVGDRVTITCKASQNINRYLNWYQQKPGKAPKLLIYIANSLQTGVPSRFSGSGSGTDFTLTISSLQPEDFATYYCLQHNSWYTFGQGTKLEIK

SEQ ID NO: 60 - protein, 106 aa, synthetic construct
DIQMTQSPSSLSASVGDRVTITCKASQNINRYLNWYQQKPGKAPKLLISIANSLQTGVPSRFSGSGSGTDFTLTISSLQPEDFATYYCLQHNSWYTFGQGTKLEIK

SEQ ID NO: 61 - protein, 106 aa, synthetic construct
DIQMTQSPSSLSASVGDRVTITCKASQNINRYLNWYQQKPGKAPKLLISIANSLQTGIPSRFSGSGSGTDFTLTISSLQPEDFATYYCLQHNSWYTFGQGTKLEIK

SEQ ID NO: 62 - protein, 106 aa, synthetic construct
DIQMTQSPSSLSASVGDRVTITCKASQNINRYLNWYQQKPGKAPKVLISIANSLQTGIPSRFSGSGSGTDFTLTISSLQPEDFATYFCLQHNSWYTFGQGTKLEIK

SEQ ID NO: 63 - protein, 98 aa, synthetic construct; identical to SEQ ID NO: 33

SEQ ID NO: 64 - protein, 16 aa, synthetic construct
RIRYNADTSYNSALKS

SEQ ID NO: 65 - protein, 16 aa, synthetic construct
RIRYNGDTSYNAALKS

SEQ ID NO: 66 - protein, 16 aa, synthetic construct
RIRYNADTSYNAALKS

SEQ ID NO: 67 - protein, 8 aa, synthetic construct
LQHGAGYT

SEQ ID NO: 68 - protein, 16 aa, synthetic construct
RVRYNADTSYNSALKS

SEQ ID NO: 69 - protein, 16 aa, synthetic construct
RVRYNGDTSYNAALKS

SEQ ID NO: 70 - protein, 16 aa, synthetic construct
RVRYNADTSYNAALKS

SEQ ID NO: 71 - protein, 8 aa, synthetic construct
LQHNAGYT

SEQ ID NO: 72 - protein, 16 aa, synthetic construct
RMRYNADTSYNSALKS

SEQ ID NO: 73 - protein, 16 aa, synthetic construct
RMRYNGDTSYNAALKS

SEQ ID NO: 74 - protein, 16 aa, synthetic construct
RMRYNADTSYNAALKS

SEQ ID NO: 75 - protein, 7 aa, synthetic construct
IANALQT

SEQ ID NO: 76 - protein, 8 aa, synthetic construct
LQHNAWYT

SEQ ID NO: 77 - protein, 255 aa, synthetic construct
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 78 - protein, 455 aa, synthetic construct
EVQLVESGGGLVQPKGSLKLSCAASGFTFNTYAMNWVRQAPGKGLEWVARIRSKYNNYATYYADSVKDRFTISRDDSQSILYLQMNNLKTEDTAMYYCVRHGNFGNSYVSWFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 79 - protein, 215 aa, synthetic construct
QAVVTQESALTTSPGETVTLTCRSSTGAVTTSNYANWVQEKPDHLFTGLIGGTNKRAPGVPARFSGSLIGDKAALTITGAQTEDEAIYFCALWYSNLWVFGGGTKLTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS